(19) United States
(12) Patent Application Publication
(10) Pub. No.: US 2009/0099147 A1
(43) Pub. Date: Apr. 16, 2009

(54) NON-STEROIDAL PROGESTERONE RECEPTOR MODULATORS

(76) Inventors: Wolfgnag SCHWEDE, Glienicke (DE); Carsten Moeller, Berlin (DE); Anja Schmidt, Orange, NJ (US); Ulrike Fuhrmann, Berlin (DE); Andrea Rotgeri, Berlin (DE); Thomas Kirkland, Atascadero, CA (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/170,035

(22) Filed: Jul. 9, 2008

Related U.S. Application Data
(60) Provisional application No. 60/948,763, filed on Jul. 10, 2007.

(51) Int. Cl. A61K 31/565; C07C 233/15; A61K 31/167; C07D 333/54; A61P 15/00; A61K 31/381; C07D 241/04

(52) U.S. Cl. 514/171; 564/174; 514/622; 514/443; 549/53; 544/400; 514/252.12

(57) ABSTRACT

The present invention relates to non-steroidal progesterone receptor modulators of the general formula I

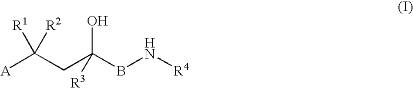

the use of the progesterone receptor modulators for producing medicaments, and pharmaceutical compositions which comprise these compounds.

The compounds according to the invention are suitable for the therapy and prophylaxis of gynaecological disorders such as endometriosis, leiomyomas of the uterus, dysfunctional bleeding and dysmenorrhoea, and for the therapy and prophylaxis of hormone-dependent tumours and for use for female fertility control and for hormone replacement therapy.

This application claims the benefit of the filing date of U.S. Provisional Application Ser. No. 60/948,763 filed Jul. 10, 2007.

The present invention relates to non-steroidal progesterone receptor modulators, to a process for their preparation, to the use of the progesterone receptor modulators for producing medicaments, and to pharmaceutical compositions which comprise these compounds.

The steroid hormone progesterone controls in a decisive manner the reproductive process in the female body. Progesterone is secreted in large quantities during the cycle and pregnancy respectively by the ovary and the placenta. Progesterone in cooperation with oestrogens brings about cyclic changes in the uterine mucosa (endometrium) during the menstrual cycle. Elevated progesterone levels after ovulation influence the uterine mucosa to convert it into a state permitting nidation of an embryo (blastocyst). During pregnancy, progesterone controls the relaxation of the myometrium and maintains the function of the decidual tissue.

It is further known that progesterone inhibits endometrial proliferation by suppressing oestrogen-mediated mitosis in uterine tissue (K. Chwalisz, R. M. Brenner, U. Fuhrmann, H. Hess-Stumpp, W. Elger, Steroids 65, 2000, 741-751).

Progesterone and progesterone receptors are also known to play a significant part in pathophysiological processes. Progesterone receptors have been detected in the foci of endometriosis, but also in tumours of the uterus, of the breast and of the CNS. It is further known that uterine leiomyomas grow progesterone-dependently.

The effects of progesterone in the tissues of the genital organs and in other tissues occur through interactions with progesterone receptors which are responsible for the cellular effects.

Progesterone receptor modulators are either pure agonists or inhibit the effect of progesterone partly or completely. Accordingly, substances are defined as pure agonists, partial agonists (selective progesterone receptor modulators=SPRMs) and pure antagonists.

In accordance with the ability of progesterone receptor modulators to take effect via the progesterone receptor, these compounds have a considerable potential as therapeutic agents for gynaecological and oncological indications and for obstetrics and fertility control.

Pure progesterone receptor antagonists completely inhibit the effect of progesterone on the progesterone receptor. They have anti-ovulatory properties and the ability to inhibit oestrogen effects in the endometrium, as far as complete atrophy. They are therefore particularly suitable for intervening in the female reproductive process, e.g. post-ovulation, in order to prevent nidation of a fertilized egg cell, during pregnancy in order to increase the reactivity of the uterus to prostaglandins or oxytocin, or in order to achieve opening and softening (“ripening”) of the cervix, and to induce a great readiness of myometrium to contract.

A beneficial effect on the pathological event is expected in foci of endometriosis and in tumour tissues which are equipped with progesterone receptors after administration of pure progesterone receptor antagonists. There might be particular advantages for influencing pathological states such as endometriosis or uterine leiomyomas if ovulation inhibition can additionally be achieved by the progesterone receptor antagonists. Ovulation inhibition also dispenses with some of the ovarian hormone production and thus the stimulating effect, deriving from this proportion, on the pathologically altered tissue.

The first progesterone receptor antagonist described, RU 486 (also mifepristone), was followed by the synthesis and characterization of a large number of analogues with progesterone receptor-antagonistic activity of varying strength. Whereas RU 486 shows an antiglucocorticoid effect in addition to the progesterone receptor-antagonistic effect, compounds synthesized later are notable in particular for a more selective effect as progesterone receptor antagonists.

Besides steroidal compounds such as onapristone or lilopristone, which are notable by comparison with RU 486 for a better dissociation of the progesterone receptor-antagonistic effect and the antiglucocorticoid effect, also known from the literature are various non-steroidal structures whose antagonistic effect on the progesterone receptor is being investigated [see, for example, S. A. Leonhardt and D. P. Edwards, Exp. Biol. Med. 227: 969-980 (2002) and R. Winneker, A. Fensome, J. E. Wrobel, Z. Zhang, P. Zhang, Seminars in Reproductive Medicine, Volume 23: 46-57 (2005)]. However, non-steroidal compounds disclosed to date have only moderate antagonistic activity compared with the known steroidal structures. The most effective non-steroidal compounds are reported to have in vitro activities which are 10% of the activity of RU 486.

The antiglucocorticoid activity is disadvantageous for therapeutic use, where the inhibition of progesterone receptors is at the forefront of the therapy. An antiglucocorticoid activity causes unwanted side effects at the dosages necessary for therapy. This may prevent administration of a therapeutically worthwhile dose or lead to discontinuation of the treatment.

Partial or complete reduction of the antiglucocorticoid properties is therefore an important precondition for therapy with progesterone receptor antagonists, especially for those indications requiring treatment lasting weeks or months.

In contrast to the pure antagonists, partial progesterone receptor agonists (SPRMs) show a residual agonistic property which may vary in strength. This leads to these substances showing agonistic effects on the progesterone receptor in certain organ systems (D. DeManno, W. Elger, R. Garg, R. Lee, B. Schneider, H. Hess-Stumpp, G. Schuber, K. Chwalisz, Steroids 68, 2003, 1019-1032). Such an organ-specific and dissociated effect may be of therapeutic benefit for the described indications.

It is therefore an object of the present invention to provide further non-steroidal progesterone receptor modulators. These compounds are intended to have a reduced antiglucocorticoid effect and therefore be suitable for the therapy and prophylaxis of gynaecological disorders such as endometriosis, leiomyomas of the uterus, dysfunctional bleeding and dysmenorrhoea. The compounds according to the invention are additionally intended to be suitable for the therapy and prophylaxis of hormone-dependent tumours, for example of breast, endometrial, ovarian and prostate carcinomas. The compounds are intended furthermore to be suitable for use in female fertility control and for female hormone replacement therapy.

The object is achieved according to the present invention by the provision of non-steroidal compounds of the general formula I

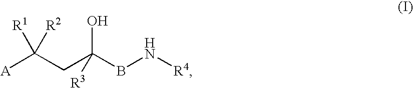

in which
R¹ and R² are independently of one another a hydrogen atom, a branched or unbranched C₁-C₅-alkyl group, further forming together with the C atom of the chain a ring having a total of 3-7 members,
R³ is a radical C≡C—R^{a}, where
   R^{a} is a hydrogen or a C₁-C₈-alkyl, C₂-C₈-alkenyl, C₂-C₈-alkynyl, C₃-C₁₀-cycloalkyl, 3-8-membered heterocycloalkyl optionally substituted one or more times, identically or differently, by K, or C₆-C₁₂-aryl or 3-8-membered heteroaryl optionally substituted one or more times, identically or differently, by L, or silicon
      K is a cyano, halogen, hydroxy, nitro, azido, —C(O)R^{b}, CO₂R^{b}, —O—R^{b}—OSiR^{b}R^{c}R^{d}—S—R^{b}, SO₂NR^{c}R^{d}, —C(O)—NR^{c}R^{d}, —OC(O)—NR^{c}R^{d}, —C═NOR^{b}—NR^{c}R^{d} or C₃-C₁₀-cycloalkyl, 3-8-membered heterocycloalkyl optionally substituted one or more times, identically or differently, by M, or C₆-C₁₂-aryl or 3-8-membered heteroaryl optionally substituted one or more times, identically or differently, by L,
      L is C₁-C₈-alkyl, C₂-C₈-alkenyl, C₂-C₈-alkynyl, C₁-C₆-perfluoroalkyl, C₁-C₆-perfluoroalkoxy, C₁-C₆-alkoxy-C₁-C₆-alkoxy, (CH₂)ₚ—C₃-C₁₀-cycloalkyl, (CH₂)ₚ-heterocycloalkyl, (CH₂)ₚCN, (CH₂)ₚHal, (CH₂)ₚNO₂, (CH₂)ₚ—C₆-C₁₂-aryl, (CH₂)ₚ-heteroaryl, —(CH₂)ₚPO₃(R^{b})₂,
         —(CH₂)ₚNR^{c}R^{d}, —(CH₂)ₚNR^{e}COR^{b}, —(CH₂)ₚNR^{e}CSR^{b}, —(CH₂)ₚNR^{e}S(O)R^{b}, —(CH₂)ₚNR^{e}S(O)₂R^{b}, —(CH₂)ₚNR^{e}CONR^{c}R^{d}, —(CH₂)ₚNR^{e}COOR^{b}, —(CH₂)ₚNR^{e}C(NH)NR^{c}R^{d}, —(CH₂)ₚNR^{e}CSNR^{c}R^{d}, (CH₂)ₚNR^{e}S(O)NR^{c}R^{d}, —(CH₂)ₚNR^{e}S(O)₂NR^{c}R^{d}, —(CH₂)ₚCOR^{b}, —(CH₂)ₚCSR^{b}, —(CH₂)ₚS(O)R^{b}, —(CH₂)ₚS(O)(NH)R^{b}, —(CH₂)ₚS(O)₂R^{b}, —(CH₂)ₚS(O)₂NR^{c}R^{d}, —(CH₂)ₚSO₂OR^{b}, —(CH₂)ₚCO₂R^{b}, —(CH₂)ₚCONR^{c}R^{d}, —(CH₂)ₚCSNR^{c}R^{d}, —(CH₂)ₚOR^{b}, —(CH₂)ₚOCOR^{b}, —(CH₂)ₚSR^{b}, —(CH₂)ₚCR^{b}(OH)—R^{e}, —(CH₂)ₚ—C═NOR^{b}, —O—(CH₂)ₙ—O—, —O—(CH₂)ₙ—CH₂—, —O—CH═CH— or —(CH₂)ₙ+₂—, where n is 1 or 2, and the terminal oxygen atoms and/or carbon atoms are linked to directly adjacent ring carbon atoms,
      M is C₁-C₆-alkyl or a group —COR^{b}, CO₂R^{b}, —O—R^{b}, or —NR^{c}R^{d}, where
         R^{b} is a hydrogen or a C₁-C₆-alkyl, C₂-C₈-alkenyl, C₂-C₈-alkynyl, C₃-C₁₀-cycloalkyl, C₆-C₁₂-aryl or C₁-C₃-perfluoroalkyl and
         R^{c} and R^{d} are independently of one another a hydrogen, C₁-C₆-alkyl, C₂-C₈-alkenyl, C₂-C₈-alkynyl, C₃-C₁₀-cycloalkyl, C₆-C₁₂-aryl, C(O)R^{b} or a hydroxy group, where if
         R^{c} is a hydroxy group, then R^{d} can only be a hydrogen, a C₁-C₆-alkyl, C₂-C₈-alkenyl, C₂-C₈-alkynyl, C₃-C₁₀-cycloalkyl or C₆-C₁₂-aryl and vice versa, and
         R^{e} is a hydrogen, C₁-C₆-alkyl, C₂-C₈-alkenyl, C₂-C₈-alkynyl, C₃-C₁₀-cycloalkyl or C₆-C₁₂-aryl, and
         p can be a number from 0-6,
            or
R³ is a radical C═C—R^{g}R^{h}, where
   R^{g} and R^{h} are independently of one another a hydrogen or a C₁-C₈-alkyl, C₂-C₈-alkenyl or C₂-C₈-alkynyl optionally substituted one or more times, identically or differently, by X, in which
      X is a cyano, halogen, hydroxy, nitro, —C(O)R^{b}, CO₂R^{b}, —O—R^{b}, —C(O)—NR^{c}R^{d}, —NR^{c}R^{d} with the meanings already mentioned before for R^{b}, R^{c} and R^{d}, and
R⁴ may be a 3-8-membered aromatic or heteroaromatic mono- or bicycle which is unsubstituted or optionally substituted by 1-3 radicals, or one of the following groups:
   A: 6-membered/6-membered ring systems:

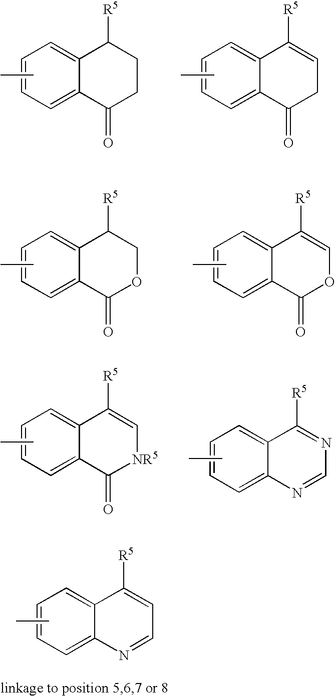

B: 6-membered/5-membered ring systems:

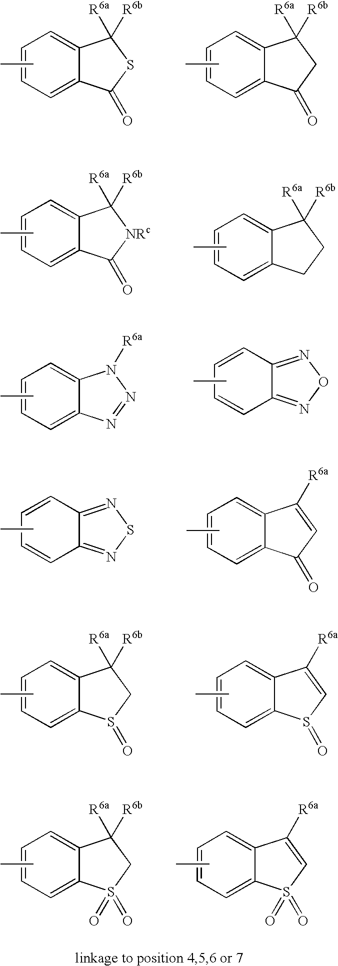

R⁵ may be hydrogen or C₁-C₄ alkyl or C₁-C₄ perfluoroalkyl,
R^{6a} and R^{6b} are independently of one another a hydrogen atom, a C₁-C₄-alkyl, a C₂-C₄-alkenyl or forming together with the ring carbon atom a 3-6-membered ring,
A is a mono- or bicyclic carbocyclic or heterocyclic aromatic ring which may optionally be substituted one or more times by C₁-C₈-alkyl, C₂-C₈-alkenyl, C₂-C₈-alkynyl, C₁-C₆-perfluoroalkyl, C₁-C₆-perfluoroalkoxy, C₁-C₆-alkoxy-C₁-C₆-alkyl, C₁-C₆-alkoxy-C₁-C₆-alkoxy, (CH₂)ₚ—C₃-C₁₀-cycloalkyl, (CH₂)ₚ-heterocycloalkyl, (CH₂)ₚCN, (CH₂)ₚHal, (CH₂)ₚNO₂, (CH₂)ₚ—C₆-C₁₂-aryl, (CH₂)ₚ-heteroaryl, —(CH₂)ₚPO₃(R^{b})₂, —(CH₂)ₚNR^{c}R^{d}, —(CH₂)ₚNR^{e}COR^{b}, —(CH₂)ₚNR^{e}CSR^{b}, —(CH₂)ₚNR^{e}S(O)R^{b}, —(CH₂)ₚNR^{e}S(O)₂R^{b}, —(CH₂)ₚNR^{e}CONR^{c}R^{d}, —(CH₂)ₚNR^{e}COOR^{b}, —(CH₂)ₚNR^{e}C(NH)NR^{c}R^{d}, —(CH₂)ₚNR^{e}CSNR^{c}R^{d}, —(CH₂)ₚNR^{e}S(O)NR^{c}R^{d}, —(CH₂)ₚNR^{e}S(O)₂NR^{c}R^{d}, —(CH₂)ₚCOR^{b}, —(CH₂)ₚCSR^{b}, —(CH₂)ₚS(O)R^{b}, —(CH₂)ₚS(O)(NH)R^{b}, —(CH₂)ₚS(O)₂R^{b}, —(CH₂)ₚS(O)₂NR^{c}R^{d}, —(CH₂)ₚSO₂OR^{b}, —(CH₂)ₚCO₂R^{b}, —(CH₂)ₚCONR^{c}R^{d}, —(CH₂)ₚCSNR^{c}R^{d}, —(CH₂)ₚOR^{b}, —(CH₂)ₚSR^{b}, —(CH₂)ₚCR^{b}(OH)—R^{d}, (CH₂)—C═NOR^{b}, —O—(CH₂)ₙ—O—, —O—(CH₂)ₙ—CH₂—, —O—CH═CH— or —(CH₂)ₙ+₂—, where n is 1 or 2, and the terminal oxygen atoms and/or carbon atoms are linked to directly adjacent ring carbon atoms, or
A is a radical —CO₂R^{b}, C(O)NR^{c}R^{d}, COR^{b},
   or
A is an alkenyl group —CR⁵═CR⁶R⁷, where
   R⁵, R⁶ and R⁷ are identical or different and are independently of one another hydrogen atoms, halogen atoms, aryl radicals or an unsubstituted or partly or completely fluorinated C₁-C₅-alkyl group, or
A is an alkynyl group —C≡CR⁵, with the meaning stated above for R⁵, and
B is a carbonyl or a CH₂ group,
   and their pharmaceutically acceptable salts.

The compounds according to the invention of the general formula (I) may, owing to the presence of centres of asymmetry, exist as different stereoisomers. Both the racemates and the separate stereoisomers belong to the subject matter of the present invention.

The present invention further includes the novel compounds as active pharmaceutical ingredients, the preparation thereof, their therapeutic use and pharmaceutical dosage forms which comprise the novel substances.

The compounds according to the invention of the general formula (I) or their pharmaceutically acceptable salts can be used to produce a medicament, in particular for the treatment and prophylaxis of gynaecological disorders such as endometriosis, leiomyomas of the uterus, dysfunctional bleeding and dysmenorrhoea. The compounds according to the invention may further be used for the treatment and prophylaxis of hormone-dependent tumours such as, for example, for breast, prostate and endometrial carcinoma.

The compounds according to the invention of the general formula (I) or their pharmaceutically acceptable salts are suitable for use for female fertility control or for female hormone replacement therapy.

The non-steroidal compounds according to the invention of the general formula I have strong antagonistic or strong partial agonistic effects on the progesterone receptor. They show a strong dissociation of effects in relation to their strength of binding to the progesterone receptor and to the glucocorticoid receptor. Whereas known progesterone receptor antagonists such as mifepristone (RU 486) show, besides the desired high binding affinity for the progesterone receptor, likewise a high affinity for the glucocorticoid receptor, the compounds according to the invention are notable for a very low glucocorticoid receptor binding with simultaneously a high progesterone receptor affinity.

The substituents, defined as groups, of the compounds according to the invention of the general formula I may in each case have the following meanings:

C₁-C₅—, C₁-C₆- and C₁-C₈-alkyl group means linear or nonlinear, branched or unbranched alkyl radicals. Examples thereof are a methyl, ethyl, n-propyl, isopropyl, n-, iso-, tert-butyl, an n-pentyl, 2,2-dimethylpropyl, 3-methylbutyl, hexyl, heptyl or octyl group.

Preferred in the meaning of R^{a} in this connection are the methyl, ethyl, n-propyl or n-butyl group and an n-pentyl group.

Preferred in the meaning of R^{a} and R² are methyl or ethyl.

A hydrogen is preferred according to the invention for R^{4a} and R^{4b}.

Alkenyl means branched or unbranched alkenyl radicals. Examples of the meaning of a C₂-C₈-alkenyl group in the context of the invention are the following: vinyl, allyl, 3-buten-1-yl or 2,3-dimethyl-2-propenyl. If the aromatic system A is substituted by a C₂-C₈-alkenyl radical, it is preferably a vinyl group.

Alkynyl means branched or unbranched alkynyl radicals. A C₂-C₈-alkynyl radical is intended to be for example an ethynyl, propynyl, butynyl, pentynyl, hexynyl and octynyl group, preferably an ethynyl or propynyl group.

3-10-Membered cycloalkyl or heterocycloalkyl means both monocyclic and bicyclic radicals.

Examples which may be mentioned of monocyclic C₃-C₁₀-cycloalkyl in the meaning of R³, K, L, R^{b}, R^{c}, R^{d}, R⁴, R^{6a} and R^{6b} are cyclopropane, cyclobutane, cyclopentane and cyclohexane. Cyclopropyl, cyclopentyl and cyclohexyl are preferred.

Heterocycloalkyl in the meaning of R^{a}, K and L means 3-8-membered monocyclic heterocycloalkyl radicals. Examples of heterocycloalkyl are morpholine, tetrahydrofurane, pyrane, piperazine, piperidine, pyrrolidine, oxirane, oxetane, aziridine, dioxolane and dioxane, it being possible to use any chemically reasonable isomer in relation to the positions of the heteroatoms.

Possible examples of C₁-C₆-alkoxyl-C₁-C₆-alkoxy group are methoxymethoxy, ethoxymethoxy or 2-methoxyethoxy.

A radical OR^{b} in the context of the invention is a hydroxy, methoxy, ethoxy, n-propoxy, isopropoxy, n-, iso-, tert-butoxy or n-pentoxy, 2,2-dimethylpropoxy or 3-methylbutoxy group. Hydroxy, methoxy and ethoxy are preferred.

Suitable for a partly or completely fluorinated C₁-C₅-alkyl group are the perfluorinated alkyl groups above. Of these, preference is given in particular to the trifluoromethyl or pentafluoroethyl group and, partly fluorinated alkyl groups, for example the 5,5,4,4-pentafluoropentyl or 5,5,5,4,4,3,3-heptafluoropentyl group.

Suitable C₁-C₃- and C₁-C₆-perfluoroalkyl groups are likewise in particular trifluoromethyl or the pentafluoroethyl group.

Preferred C₁-C₃- and C₁-C₆-perfluoroalkoxy groups are the trifluoromethoxy or pentafluoroethoxy radical.

A halogen atom may be a fluorine, chlorine, bromine or iodine atom. Fluorine, chlorine or bromine is preferred here.

If R¹ and R² form together with the C atom of the chain a 3-7 membered ring, this is for example a cyclopropyl, -butyl, -pentyl or -hexyl ring. The cyclopropyl and the cyclopentyl ring are preferred.

The mono- or bicyclic carbocyclic aromatic ring A, which may be substituted more than once, is a carbocyclic or heterocyclic aryl radical.

In the former case it is for example a phenyl or naphthyl radical, preferably a phenyl radical.

It is possible to use as heterocyclic radical for example a monocyclic heterocyclic radical, for example the thienyl, furyl, pyranyl, pyrrolyl, imidazolyl, pyrazolyl, pyridyl, pyrazinyl, pyrimidinyl, pyridazinyl, thiazolyl, oxazolyl, furazanyl, pyrrolinyl, imidazolinyl, pyrazolinyl, thiazolinyl, triazolyl, tetrazolyl radical, in particular all the possible isomers in relation to the positions of the heteroatoms.

R³ means in the case of a C₆-C₁₂-aryl radical an optionally substituted phenyl, 1- or 2-naphthyl radical, with preference for the phenyl radical. Examples of a heteroaryl radical are the 2-, 3- or 4-pyridinyl, the 2- or 3-furyl, the 2- or 3-thienyl, the 2- or 3-pyrrolyl, the 2-, 4- or 5-imidazolyl, the pyrazinyl, the 2-, 4- or 5-pyrimidinyl or 3- or 4-pyridazinyl radical.

The number p for a (CH₂)ₚ radical may be an integer from 0 to 6, preferably 0, 1 or 2. “Radical” means according to the invention all functional groups mentioned under L and A in connection with (CH₂)ₚ.

In the case where the compounds of the general formula I (B═—CH₂—) are in the form of salts, this is possible for example in the form of the hydrochloride, sulphate, nitrate, tartrate, citrate, fumarate, succinate or benzoate.

If the compounds according to the invention are in the form of racemic mixtures, they can be fractionated by methods of racemate resolution familiar to the skilled person into the pure optically active forms. For example, the racemic mixtures can be separated into the pure isomers by chromatography on a support material which is itself optically active (CHIRALPAK AD®). It is also possible to esterify the free hydroxy group in a racemic compound of the general formula I with an optically active acid, and to separate the resulting diastereoisomeric esters by fractional crystallization or chromatography and to hydrolyse the separated esters in each case to the optically pure isomers. It is possible to use as optically active acid for example mandelic acid, camphorsulphonic acid or tartaric acid.

Compounds of the general formula (I) which are preferred according to the present invention are those in which:

R¹ and R² are each independently of one another a hydrogen atom, a methyl or an ethyl radical, or form together with the C atom of the chain a ring having a total of 3-7 members. Particularly preferred compounds are those in which R¹ and R² are simultaneously a hydrogen atom, a methyl or cyclopropyl radical, particularly preferably a methyl or cyclopropyl radical.

Further preferred compounds are those in which R³ is an alkynyl radical of the formula C≡C—R^{a}, where R^{a} is a C₁-C₄-alkyl, C₃-C₁₀-cycloalkyl, 3-8-membered heterocycloalkyl radical which is optionally substituted by K, or optionally a C₆-C₁₂-aryl or 3-8-membered heteroaryl radical which is substituted by L, and

K is a cyano, halogen, hydroxy, —O—R^{b}, SO₂NR^{c}R^{d}, —C(O)—NR^{c}R^{d}, NR^{c}R^{d} or a 3-8-membered heterocycloalkyl radical which is optionally substituted one or more times, identically or differently, by M, or an aryl or heteroaryl radical which is optionally substituted more than once by L, and

L is a C₁-C₄-alkyl, C₁-C₄-perfluoroalkyl, (CH₂)ₚ—C₃-C₁₀-cycloalkyl, (CH₂)ₚ-heterocycloalkyl radical, (CH₂)ₚCN, (CH₂)ₚHal, (CH₂)ₚNO₂, (CH₂)ₚ—C₆-C₁₂-aryl, (CH₂)ₚ-heteroaryl, —(CH₂)ₚNR^{c}R^{d}, or —(CH₂)ₚNR^{e}S(O)₂R^{b}, —(CH₂)ₚS(O)₂NR^{c}R^{d}, —(CH₂)ₚCONR^{c}R^{d}, —(CH₂)ₚOR^{b}, —(CH₂)ₚOCOR^{b}, —(CH₂)ₚCR^{b}(OH)—R^{e}, —(CH₂)ₚCO₂R^{b}, and

M is a C₁-C₄-alkyl radical or a group —CO₂R^{b}, —O—R^{b} or —NR^{c}R^{d}, where R^{b} is a hydrogen or a C₁-C₆-alkyl, C₃-C₁₀-cycloalkyl, C₆-C₁₂-aryl or C₁-C₃-perfluoroalkyl and

R^{c} and R^{d} are independently of one another a hydrogen atom, a C₁-C₆-alkyl, C₃-C₁₀-cycloalkyl, C₆-C₁₂-aryl, C(O)R^{b} or a hydroxy group, where if R^{c} is a hydroxy group, then R^{d} can only be a hydrogen, a C₁-C₆-alkyl, C₂-C₈-alkenyl, C₂-C₈-alkynyl, C₃-C₁₀-cycloalkyl or C₆-C₁₂-aryl, and vice versa,

and R^{e} is a hydrogen, C₁-C₆-alkyl or C₆-C₁₂-aryl, and

p may be a number, 1, 2 or 3.

Particularly preferred compounds are those in which

R^{a} is a C₁-C₄-alkyl radical which is optionally substituted by K, or a phenyl or hetaryl radical which is optionally substituted by L, where L is preferably a methyl, trifluoromethyl, methoxy, acetoxy, hydroxy, carboxyl or carboxyalkyl radical.

Additionally preferred compounds are those in which

R⁴ is a phenyl ring, particularly preferably a phenyl ring substituted by 1-3 radicals. Preferred substituents on the phenyl ring are nitro, trifluoromethyl, pentafluoroethyl, cyano, chlorine, fluorine, methyl.

Likewise preferred compounds are those in which R⁴ is one of the following groups:
A: 6-membered/6-membered ring systems:

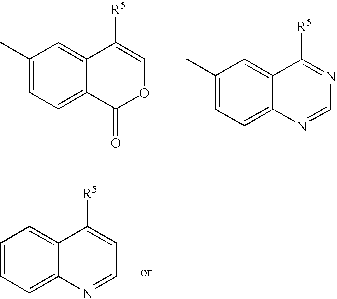

B: 6-membered/5-membered ring systems:

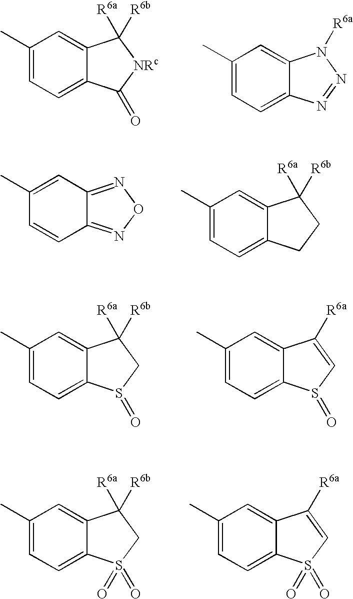

with the meanings already mentioned for R⁵ and R^{6a} and R^{6b}.

A is preferably substituted by the following radicals: C₁-C₈-alkyl, C₁-C₆-perfluoroalkyl, C₁-C₆-perfluoroalkoxy, C₁-C₆-alkoxy-C₁-C₆-alkyl, C₁-C₆-alkoxy-C₁-C₆-alkoxy, (CH₂)ₚ—C₃-C₁₀-cycloalkyl, (CH₂)ₚ-heterocycloalkyl, (CH₂)ₚCN, (CH₂)ₚHal, (CH₂)ₚNO₂, (CH₂)ₚ—C₆-C₁₂-aryl, (CH₂)ₚ-heteroaryl, —(CH₂)ₚNR^{c}R^{d}, —(CH₂)ₚNR^{e}COR^{b}, —(CH₂)ₚNR^{e}S(O)₂R^{b}, (CH₂)ₚNR^{e}CONR^{c}R^{d}, —(CH₂)ₚNR^{e}S(O)₂NR^{c}R^{d}, —(CH₂)ₚCOR^{b}, —(CH₂)ₚCSR^{b}, —(CH₂)ₚS(O)(NH)R^{b}, —(CH₂)ₚS(O)₂R^{b}, —(CH₂)ₚS(O)₂NR^{c}R^{d}, —(CH₂)ₚCO₂R^{b}, —(CH₂)ₚCONR^{c}R^{d}, —(CH₂)ₚOR^{b}, —(CH₂)ₚSR^{b}, —(CH₂)ₚCR^{b}(OH)—R^{d}, —(CH₂)ₚ—C═NOR^{b}, —O—(CH₂)ₙ—O—, —O—(CH₂)ₙ—CH₂—, —O—CH═CH— or —(CH₂)ₙ₊₂—, where n is 1 or 2, and the terminal oxygen atoms and/or carbon atoms are linked to directly adjacent ring carbon atoms.

Particularly preferred compounds are those in which A is substituted by C₁-C₄-alkyl, C₁-C₂-perfluoroalkyl, C₁-C₂-perfluoroalkoxy, (CH₂)ₚCN, (CH₂)ₚHal, —(CH₂)ₚNR^{c}R^{d}, —(CH₂)ₚS(O)(NH)R^{b}, —(CH₂)ₚS(O)₂R^{b}, —(CH₂)ₚS(O)₂NR^{c}R^{d}, —(CH₂)ₚOR^{b} or —(CH₂)ₚSR^{b} and p and R^{b}, R^{c} and R^{d} have the meanings already mentioned.

Very particularly preferred compounds are those in which A is either an unsubstituted phenyl ring or a phenyl ring which is substituted once or twice, identically or differently, by fluorine, chlorine, bromine, methyl, trifluoromethyl or methoxy.

Further preferred compounds are those in which B is a carbonyl group or a —CH₂ group.

Preferred compounds are likewise those in which p is 0 or 1.

The compounds specified below, and the use thereof, are preferred according to the invention:

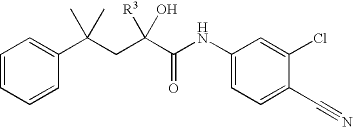

| | racemic or | |
|---|---|---|
| No. | enantiomer | R3 |
| 1 2 3 | rac+− | 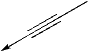 |
| 4 5 6 | rac+− | 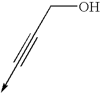 |
| 7 8 9 | rac+− | 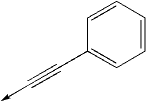 |
| 10 11 12 | rac+− | 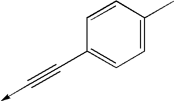 |
| 13 14 15 | rac+− | 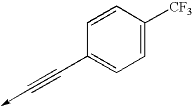 |
| 16 17 18 | rac+− | 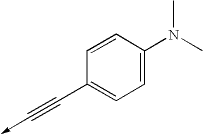 |
| 19 20 21 | rac+− | 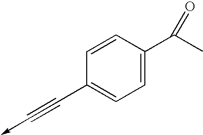 |
| 22 23 24 | rac+− | 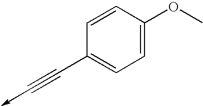 |
| 25 26 27 | rac+− | 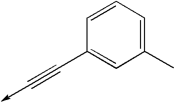 |
| 28 29 30 | rac+− | 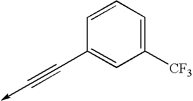 |
| 31 32 33 | rac+− | 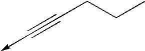 |
| 34 35 36 | rac+− | 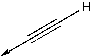 |
| 37 38 39 | rac+− | 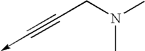 |
| 40 41 42 | rac+− | 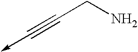 |
| 43 44 45 | rac+− | 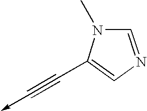 |
| 46 47 48 | rac+− | 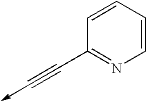 |
| 49 50 51 | rac+− | 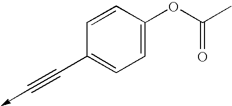 |
| 52 53 54 | rac+− | 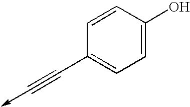 |
| 55 56 57 | rac+− | 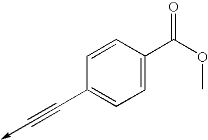 |
| 58 59 60 | rac+− | 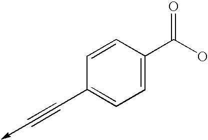 |
| 61 62 63 | rac+− | 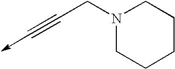 |
| 64 65 66 | rac+− | 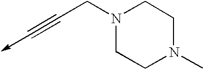 |
| 67 68 69 | rac+− | 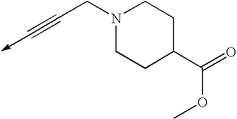 |
| 70 71 72 | rac+− | 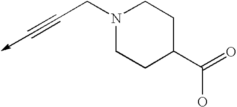 |
| 73 74 75 | rac+− | 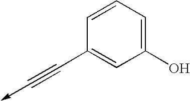 |
| 76 77 78 | rac+− | 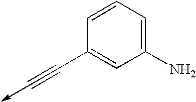 |
| 79 80 81 | rac+− | 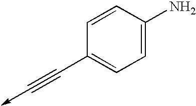 |
| 82 83 84 | rac+− | 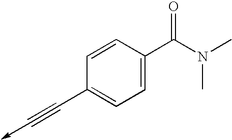 |
| 85 86 87 | rac+− | 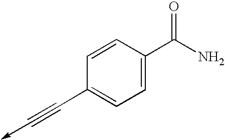 |

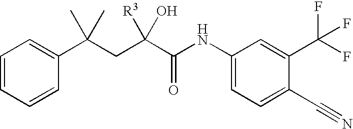

| | racemic or | |
|---|---|---|
| No. | enantiomer | R3 |
| 88 89 90 | rac+− | 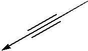 |
| 91 92 93 | rac+− | 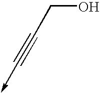 |
| 94 95 96 | rac+− | 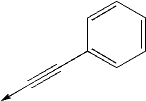 |
| 97 98 99 | rac+− | 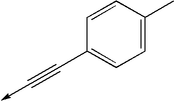 |
| 100 101 102 | rac+− | 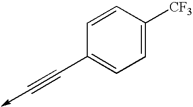 |
| 103 104 105 | rac+− | 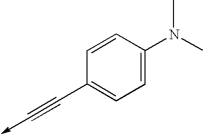 |
| 106 107 108 | rac+− | 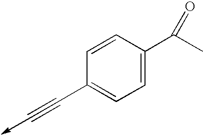 |
| 109 110 111 | rac+− | 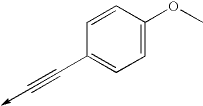 |
| 112 113 114 | rac+− | 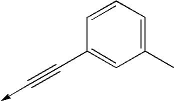 |
| 115 116 117 | rac+− | 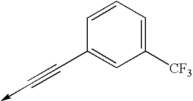 |
| 118 119 120 | rac+− | 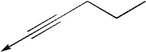 |
| 121 122 123 | rac+− | 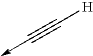 |
| 124 125 126 | rac+− | 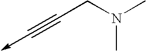 |
| 127 128 129 | rac+− | 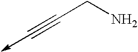 |
| 130 131 132 | rac+− | 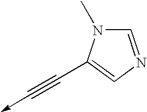 |
| 133 134 135 | rac+− | 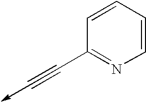 |
| 136 137 138 | rac+− | 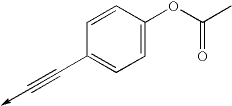 |
| 139 140 141 | rac+− | 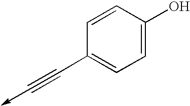 |
| 142 143 144 | rac+− | 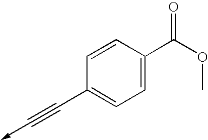 |
| 145 146 147 | rac+− | 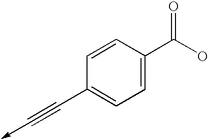 |
| 148 149 150 | rac+− | 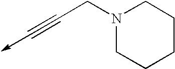 |
| 151 152 153 | rac+− | 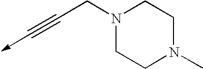 |
| 154 155 156 | rac+− | 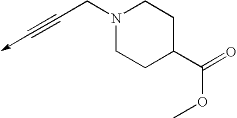 |
| 157 158 159 | rac+− | 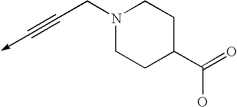 |
| 160 161 162 | rac+− | 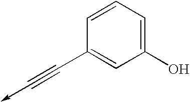 |
| 163 164 165 | rac+− | 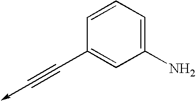 |
| 166 167 168 | rac+− | 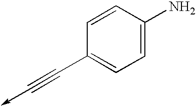 |
| 169 170 171 | rac+− | 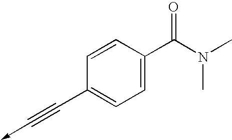 |
| 172 173 174 | rac+− | 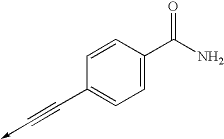 |

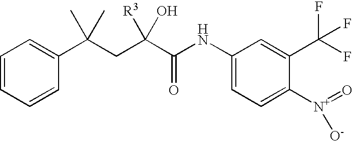

| | racemic or | |
|---|---|---|
| No. | enantiomer | R3 |
| 175 176 177 | rac+− | 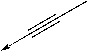 |
| 178 179 180 | rac+− | 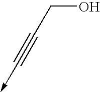 |
| 181 182 183 | rac+− | 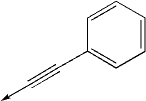 |
| 184 185 186 | rac+− | 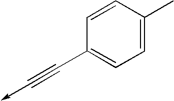 |
| 187 188 189 | rac+− | 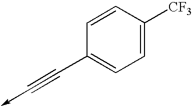 |
| 190 191 192 | rac+− | 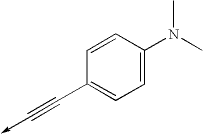 |
| 193 194 195 | rac+− | 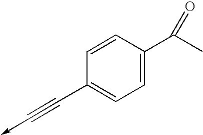 |
| 196 197 198 | rac+− | 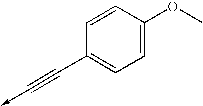 |
| 199 200 201 | rac+− | 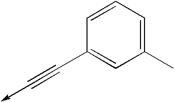 |
| 202 203 204 | rac+− | 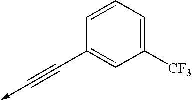 |
| 205 206 207 | rac+− | 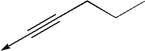 |
| 208 209 210 | rac+− | 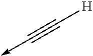 |
| 211 212 213 | rac+− | 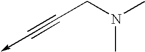 |
| 214 215 216 | rac+− | 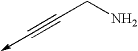 |
| 217 218 219 | rac+− | 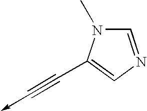 |
| 220 221 222 | rac+− | 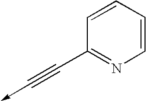 |
| 223 224 225 | rac+− | 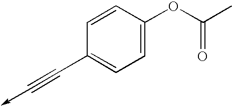 |
| 226 227 228 | rac+− | 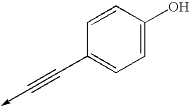 |
| 229 230 231 | rac+− | 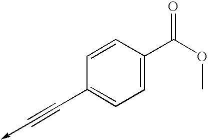 |
| 232 233 234 | rac+− | 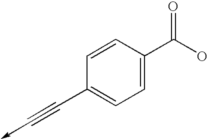 |
| 235 236 237 | rac+− | 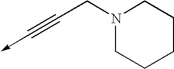 |
| 238 239 240 | rac+− | 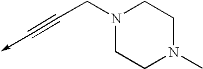 |
| 241 242 243 | rac+− | 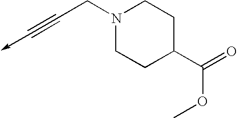 |
| 244 245 246 | rac+− | 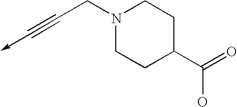 |
| 247 248 249 | rac+− | 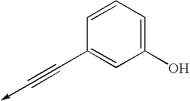 |
| 250 251 252 | rac+− | 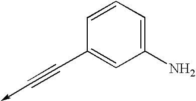 |
| 253 254 255 | rac+− | 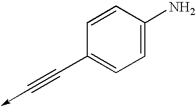 |
| 256 257 258 | rac+− | 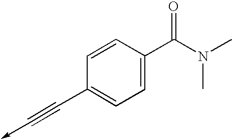 |
| 259 260 261 | rac+− | 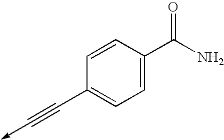 |

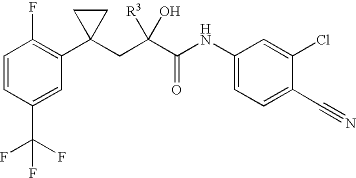

| | racemic or | |
|---|---|---|
| No. | enantiomer | R3 |
| 262 263 264 | rac+− | 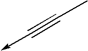 |
| 265 266 267 | rac+− | 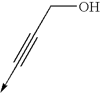 |
| 268 269 270 | rac+− | 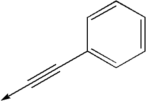 |
| 271 272 273 | rac+− | 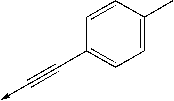 |
| 274 275 276 | rac+− | 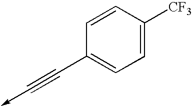 |
| 277 278 279 | rac+− | 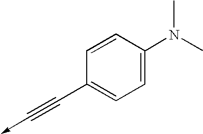 |
| 280 281 282 | rac+− | 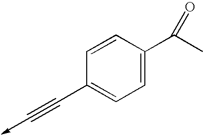 |
| 283 284 285 | rac+− | 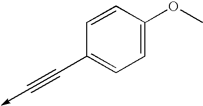 |
| 286 287 288 | rac+− | 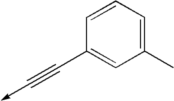 |
| 289 290 291 | rac+− | 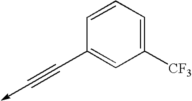 |
| 292 293 294 | rac+− | 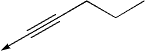 |
| 295 296 297 | rac+− | 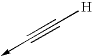 |
| 298 299 300 | rac+− | 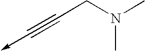 |
| 301 302 303 | rac+− | 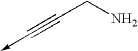 |
| 304 305 306 | rac+− | 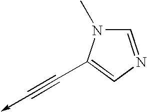 |
| 307 308 309 | rac+− | 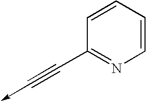 |
| 310 311 312 | rac+− | 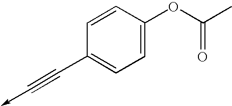 |
| 313 314 315 | rac+− | 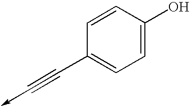 |
| 316 317 318 | rac+− | 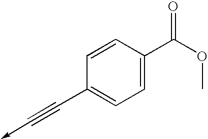 |
| 319 320 321 | rac+− | 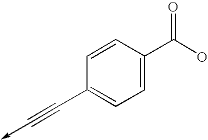 |
| 322 323 324 | rac+− | 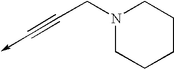 |
| 325 326 327 | rac+− | 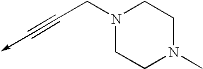 |
| 328 329 330 | rac+− | 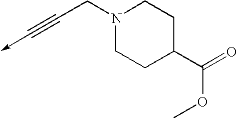 |
| 331 332 333 | rac+− | 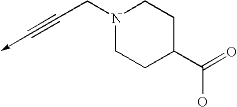 |
| 334 335 336 | rac+− | 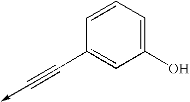 |
| 337 338 339 | rac+− | 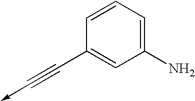 |
| 340 341 342 | rac+− | 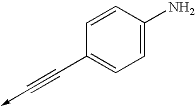 |
| 343 344 345 | rac+− | 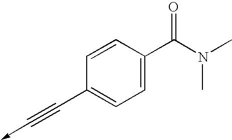 |
| 346 347 348 | rac+− | 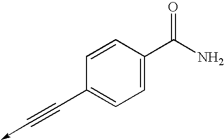 |

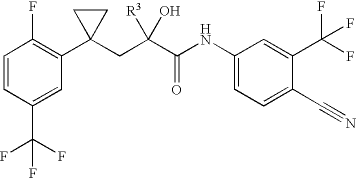

| | racemic or | |
|---|---|---|
| No. | enantiomer | R3 |
| 349 350 351 | rac+− | 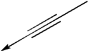 |
| 352 353 354 | rac+− | 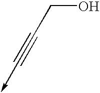 |
| 355 356 357 | rac+− | 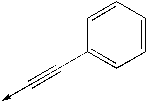 |
| 358 359 360 | rac+− | 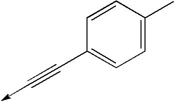 |
| 361 362 363 | rac+− | 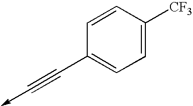 |
| 364 365 366 | rac+− | 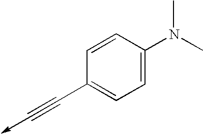 |
| 367 368 369 | rac+− | 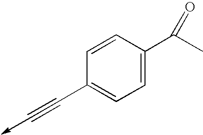 |
| 370 371 372 | rac+− | 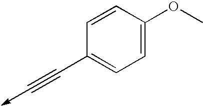 |
| 373 374 375 | rac+− | 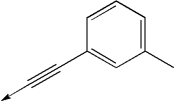 |
| 376 377 378 | rac+− | 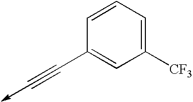 |
| 379 380 381 | rac+− | 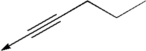 |
| 382 383 384 | rac+− | 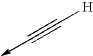 |
| 385 386 387 | rac+− | 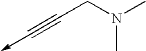 |
| 388 389 390 | rac+− | 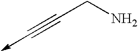 |
| 391 392 393 | rac+− | 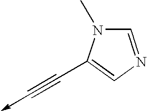 |
| 394 395 396 | rac+− | 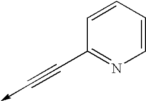 |
| 397 398 399 | rac+− | 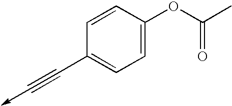 |
| 400 401 402 | rac+− | 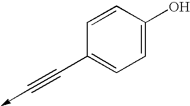 |
| 403 404 405 | rac+− | 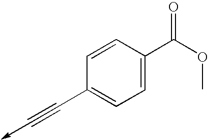 |
| 406 407 408 | rac+− | 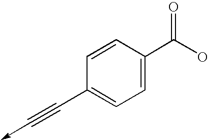 |
| 409 410 411 | rac+− | 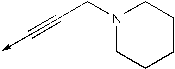 |
| 412 413 414 | rac+− | 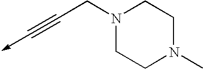 |
| 415 416 417 | rac+− | 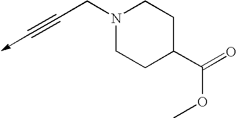 |
| 418 419 420 | rac+− | 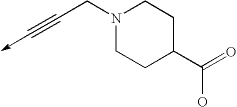 |
| 421 422 423 | rac+− | 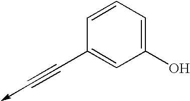 |
| 424 425 426 | rac+− | 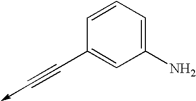 |
| 427 428 429 | rac+− | 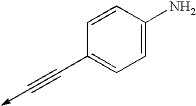 |
| 430 431 432 | rac+− | 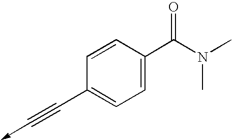 |
| 433 434 435 | rac+− | 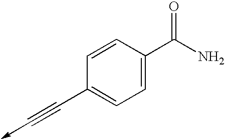 |

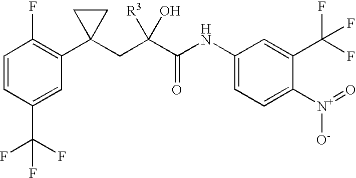

| | racemic or | |
|---|---|---|
| No. | enantiomer | R3 |
| 436 437 438 | rac+− | 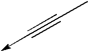 |
| 439 440 441 | rac+− | 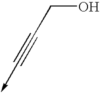 |
| 442 443 444 | rac+− | 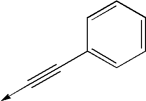 |
| 445 446 447 | rac+− | 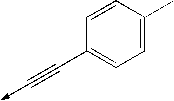 |
| 448 449 450 | rac+− | 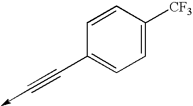 |
| 451 452 453 | rac+− | 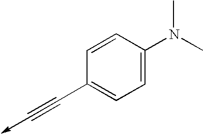 |
| 454 455 456 | rac+− | 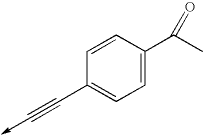 |
| 457 458 459 | rac+− | 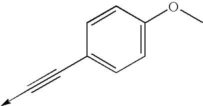 |
| 460 461 462 | rac+− | 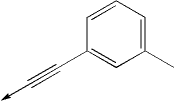 |
| 463 464 465 | rac+− | 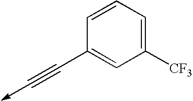 |
| 466 467 468 | rac+− | 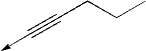 |
| 469 470 471 | rac+− | 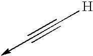 |
| 472 473 474 | rac+− | 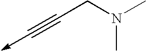 |
| 475 476 477 | rac+− | 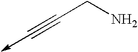 |
| 478 479 480 | rac+− | 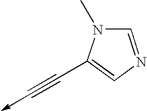 |
| 481 482 483 | rac+− | 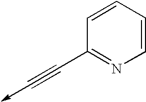 |
| 484 485 486 | rac+− | 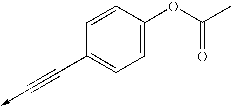 |
| 487 488 489 | rac+− | 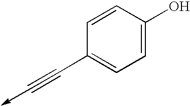 |
| 490 491 492 | rac+− | 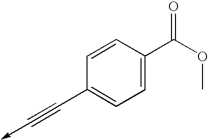 |
| 493 494 495 | rac+− | 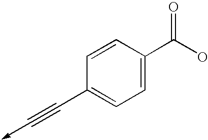 |
| 496 497 498 | rac+− | 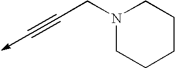 |
| 499 500 501 | rac+− | 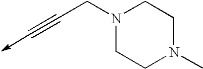 |
| 502 503 504 | rac+− | 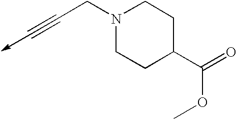 |
| 505 506 507 | rac+− | 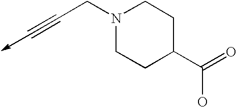 |
| 508 509 510 | rac+− | 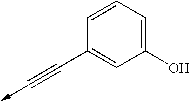 |
| 511 512 513 | rac+− | 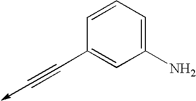 |
| 514 515 516 | rac+− | 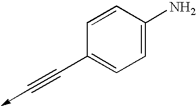 |
| 517 518 519 | rac+− | 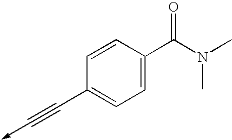 |
| 520 521 522 | rac+− | 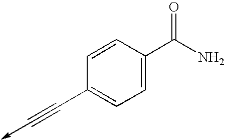 |

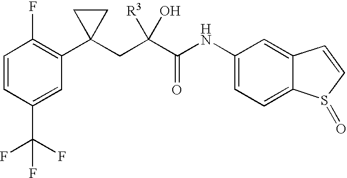

| | racemic or | |
|---|---|---|
| No. | enantiomer | R3 |
| 523 524 525 | rac+− | 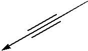 |
| 526 527 528 | rac+− | 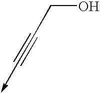 |
| 529 530 531 | rac+− | 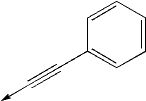 |
| 532 533 534 | rac+− | 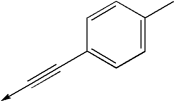 |
| 535 536 537 | rac+− | 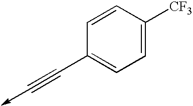 |
| 538 539 540 | rac+− | 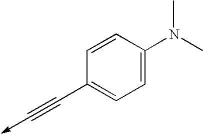 |
| 541 542 543 | rac+− | 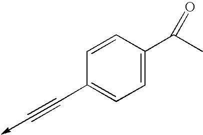 |
| 544 545 546 | rac+− | 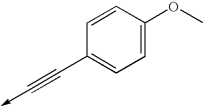 |
| 547 548 549 | rac+− | 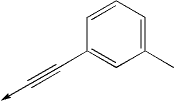 |
| 550 551 552 | rac+− | 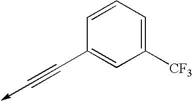 |
| 553 554 555 | rac+− | 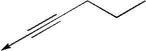 |
| 556 557 558 | rac+− | 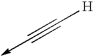 |
| 559 560 561 | rac+− | 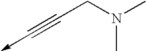 |
| 562 563 564 | rac+− | 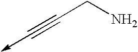 |
| 565 566 567 | rac+− | 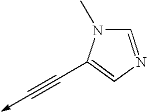 |
| 568 569 570 | rac+− | 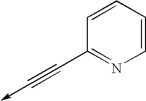 |
| 571 572 573 | rac+− | 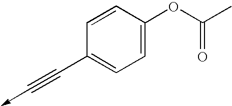 |
| 574 575 576 | rac+− | 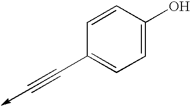 |
| 577 578 579 | rac+− | 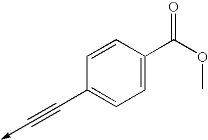 |
| 580 581 582 | rac+− | 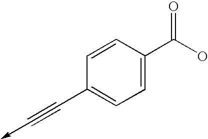 |
| 583 584 585 | rac+− | 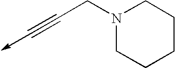 |
| 586 587 588 | rac+− | 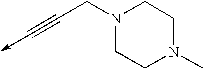 |
| 589 590 591 | rac+− | 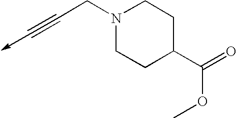 |
| 592 593 594 | rac+− | 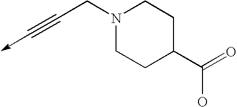 |
| 595 596 597 | rac+− | 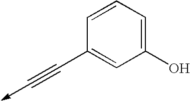 |
| 598 599 600 | rac+− | 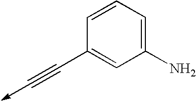 |
| 601 602 603 | rac+− | 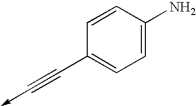 |
| 604 605 606 | rac+− | 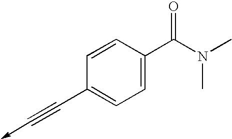 |
| 607 608 609 | rac+− | 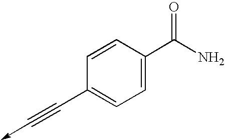 |

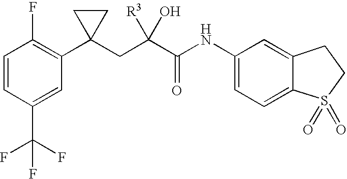

| | racemic or | |
|---|---|---|
| No. | enantiomer | R3 |
| 610 611 612 | rac+− | 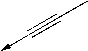 |
| 613 614 615 | rac+− | 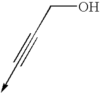 |
| 616 617 618 | rac+− | 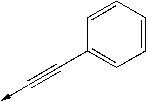 |
| 619 620 621 | rac+− | 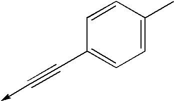 |
| 622 623 624 | rac+− | 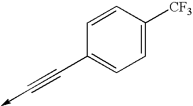 |
| 625 626 627 | rac+− | 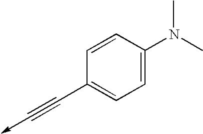 |
| 628 629 630 | rac+− | 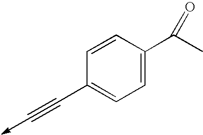 |
| 631 632 633 | rac+− | 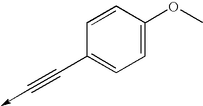 |
| 634 635 636 | rac+− | 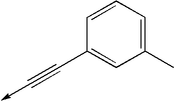 |
| 637 638 639 | rac+− | 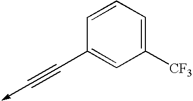 |
| 640 641 642 | rac+− | 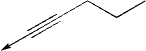 |
| 643 644 645 | rac+− | 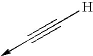 |
| 646 647 648 | rac+− | 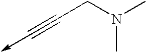 |
| 649 650 651 | rac+− | 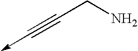 |
| 652 653 654 | rac+− | 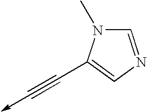 |
| 655 656 657 | rac+− | 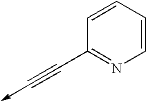 |
| 658 659 660 | rac+− | 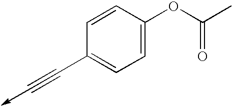 |
| 661 662 663 | rac+− | 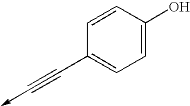 |
| 664 665 666 | rac+− | 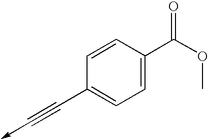 |
| 667 668 669 | rac+− | 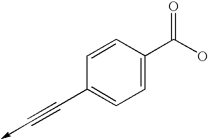 |
| 670 671 672 | rac+− | 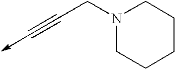 |
| 673 674 675 | rac+− | 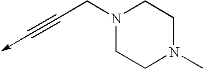 |
| 676 677 678 | rac+− | 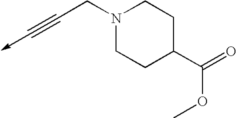 |
| 679 680 681 | rac+− | 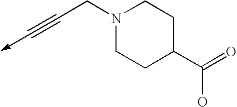 |
| 682 683 684 | rac+− | 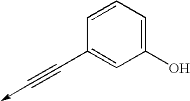 |
| 685 686 687 | rac+− | 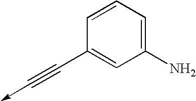 |
| 688 689 690 | rac+− | 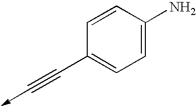 |
| 691 692 693 | rac+− | 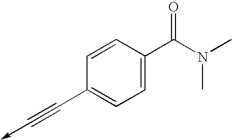 |
| 694 695 696 | rac+− | 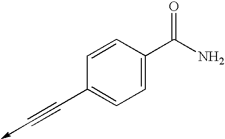 |

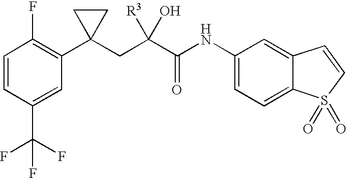

| | racemic or | |
|---|---|---|
| No. | enantiomer | R3 |
| 697 698 699 | rac+− | 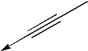 |
| 700 701 702 | rac+− | 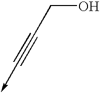 |
| 703 704 705 | rac+− | 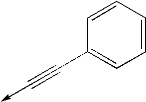 |
| 706 707 708 | rac+− | 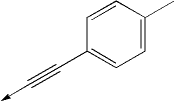 |
| 709 710 711 | rac+− | 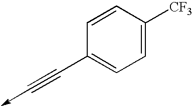 |
| 712 713 714 | rac+− | 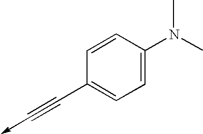 |
| 715 716 717 | rac+− | 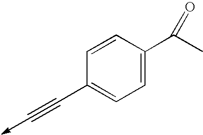 |
| 718 719 720 | rac+− | 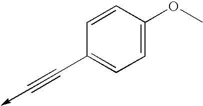 |
| 721 722 723 | rac+− | 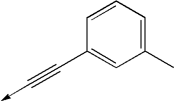 |
| 724 725 726 | rac+− | 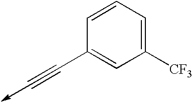 |
| 727 728 729 | rac+− | 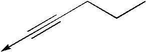 |
| 730 731 732 | rac+− | 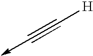 |
| 733 734 735 | rac+− | 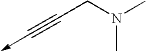 |
| 736 737 738 | rac+− | 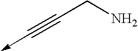 |
| 739 740 741 | rac+− | 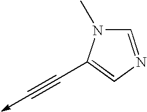 |
| 742 743 744 | rac+− | 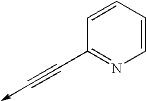 |
| 745 746 747 | rac+− | 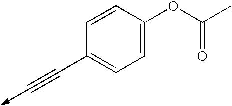 |
| 748 749 750 | rac+− | 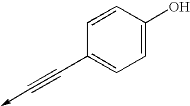 |
| 751 752 753 | rac+− | 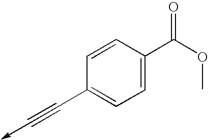 |
| 754 755 756 | rac+− | 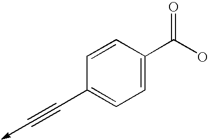 |
| 757 758 759 | rac+− | 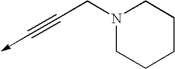 |
| 760 761 762 | rac+− | 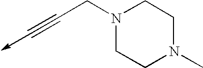 |
| 763 764 765 | rac+− | 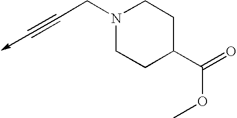 |
| 766 767 768 | rac+− | 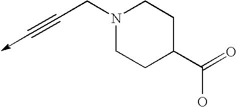 |
| 769 770 771 | rac+− | 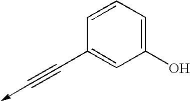 |
| 772 773 774 | rac+− | 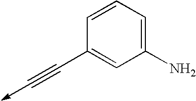 |
| 775 776 777 | rac+− | 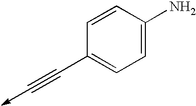 |
| 778 779 780 | rac+− | 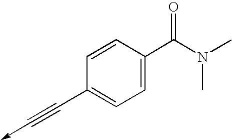 |
| 781 782 783 | rac+− | 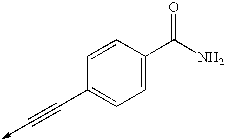 |

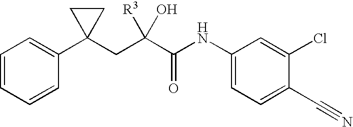

| | racemic or | |
|---|---|---|
| No. | enantiomer | R3 |
| 784 785 786 | rac+− | 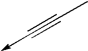 |
| 787 788 789 | rac+− | 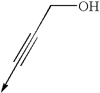 |
| 790 791 792 | rac+− | 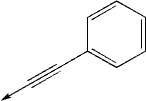 |
| 793 794 795 | rac+− | 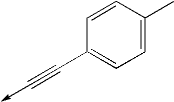 |
| 796 797 798 | rac+− | 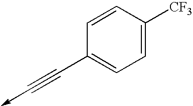 |
| 799 800 801 | rac+− | 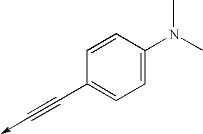 |
| 802 803 804 | rac+− | 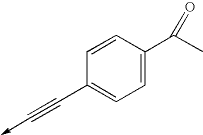 |
| 805 806 807 | rac+− | 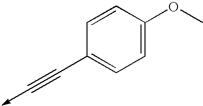 |
| 808 809 810 | rac+− | 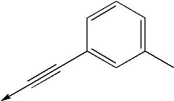 |
| 811 812 813 | rac+− | 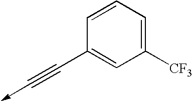 |
| 814 815 816 | rac+− | 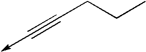 |
| 817 818 819 | rac+− | 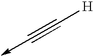 |
| 820 821 822 | rac+− | 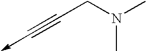 |
| 823 824 825 | rac+− | 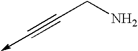 |
| 826 827 828 | rac+− | 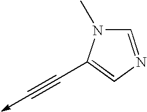 |
| 829 830 831 | rac+− | 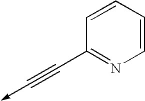 |
| 832 833 834 | rac+− | 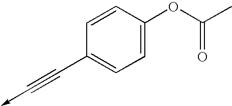 |
| 835 836 837 | rac+− | 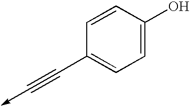 |
| 838 839 840 | rac+− | 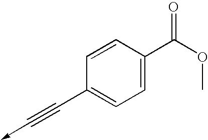 |
| 841 842 843 | rac+− | 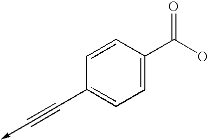 |
| 844 845 846 | rac+− | 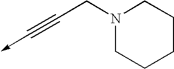 |
| 847 848 849 | rac+− | 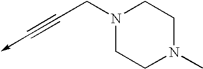 |
| 850 851 852 | rac+− | 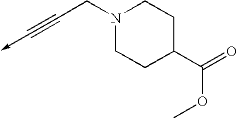 |
| 853 854 855 | rac+− | 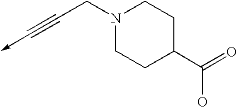 |
| 856 857 858 | rac+− | 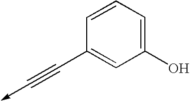 |
| 859 860 861 | rac+− | 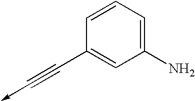 |
| 862 863 864 | rac+− | 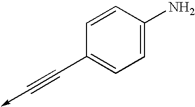 |
| 865 866 867 | rac+− | 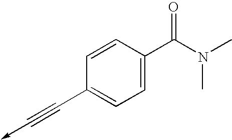 |
| 868 869 870 | rac+− | 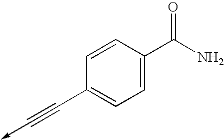 |

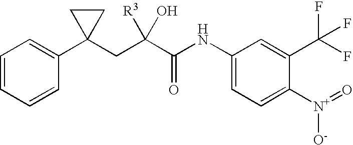

| | racemic or | |
|---|---|---|
| No. | enantiomer | R3 |
| 871 872 873 | rac+− | 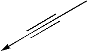 |
| 874 875 876 | rac+− | 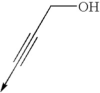 |
| 877 878 879 | rac+− | 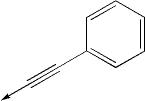 |
| 880 881 882 | rac+− | 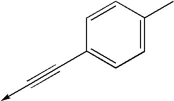 |
| 883 884 885 | rac+− | 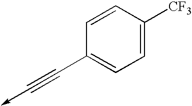 |
| 886 887 888 | rac+− | 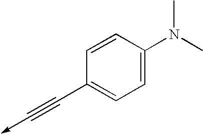 |
| 889 890 891 | rac+− | 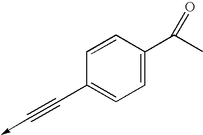 |
| 892 893 894 | rac+− | 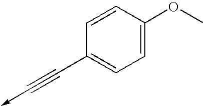 |
| 895 896 897 | rac+− | 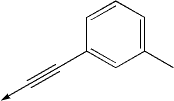 |
| 898 899 900 | rac+− | 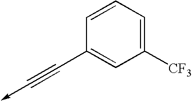 |
| 901 902 903 | rac+− | 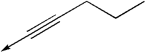 |
| 904 905 906 | rac+− | 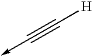 |
| 907 908 909 | rac+− | 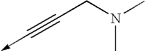 |
| 910 911 912 | rac+− | 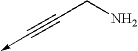 |
| 913 914 915 | rac+− | 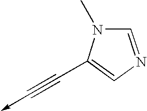 |
| 916 917 918 | rac+− | 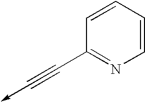 |
| 919 920 921 | rac+− | 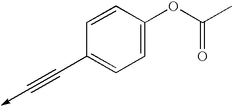 |
| 922 923 924 | rac+− | 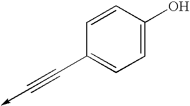 |
| 925 926 927 | rac+− | 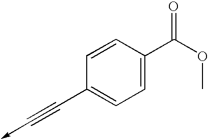 |
| 928 929 930 | rac+− | 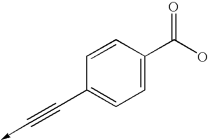 |
| 931 932 933 | rac+− | 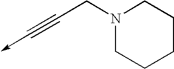 |
| 934 935 936 | rac+− | 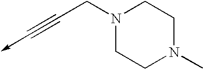 |
| 937 938 939 | rac+− | 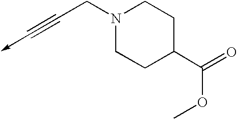 |
| 940 941 942 | rac+− | 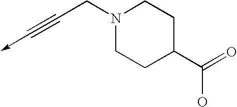 |
| 943 944 945 | rac+− | 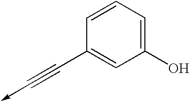 |
| 946 947 948 | rac+− | 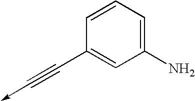 |
| 949 950 951 | rac+− | 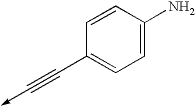 |
| 952 953 954 | rac+− | 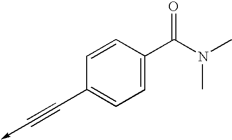 |
| 955 956 957 | rac+− | 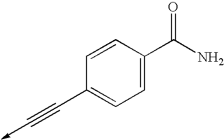 |

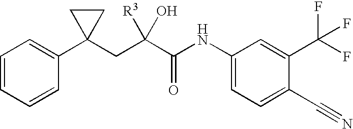

| | racemic or | |
|---|---|---|
| No. | enantiomer | R3 |
| 958 959 960 | rac+− | 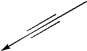 |
| 961 962 963 | rac+− | 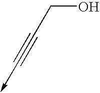 |
| 964 965 966 | rac+− | 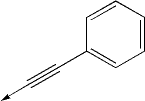 |
| 967 968 969 | rac+− | 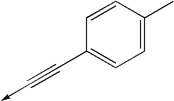 |
| 970 971 972 | rac+− | 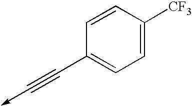 |
| 973 974 975 | rac+− | 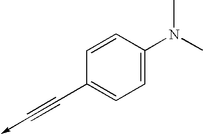 |
| 976 977 978 | rac+− | 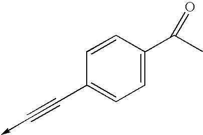 |
| 979 980 981 | rac+− | 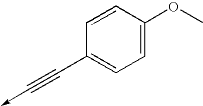 |
| 982 983 984 | rac+− | 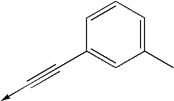 |
| 985 986 987 | rac+− | 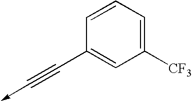 |
| 988 989 990 | rac+− | 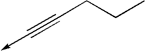 |
| 991 992 993 | rac+− | 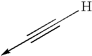 |
| 994 995 996 | rac+− | 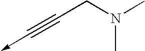 |
| 997 998 999 | rac+− | 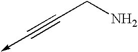 |
| 100010011002 | rac+− | 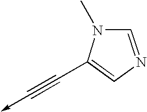 |
| 100310041005 | rac+− | 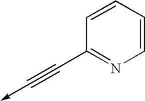 |
| 100610071008 | rac+− | 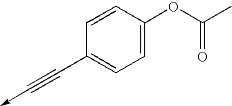 |
| 100910101011 | rac+− | 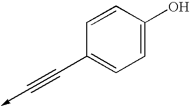 |
| 101210131014 | rac+− | 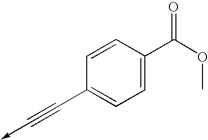 |
| 101510161017 | rac+− | 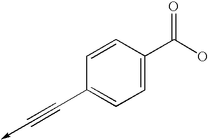 |
| 101810191020 | rac+− | 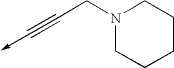 |
| 102110221023 | rac+− | 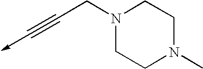 |
| 102410251026 | rac+− | 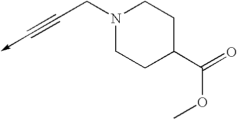 |
| 102710281029 | rac+− | 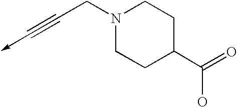 |
| 103010311032 | rac+− | 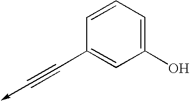 |
| 103310341035 | rac+− | 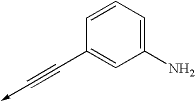 |
| 103610371038 | rac+− | 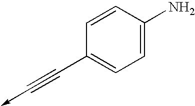 |
| 103910401041 | rac+− | 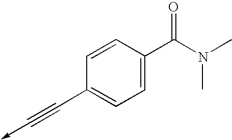 |
| 104210431044 | rac+− | 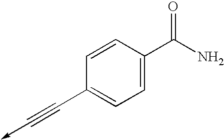 |

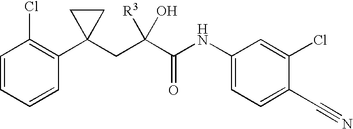

| | racemic or | |
|---|---|---|
| No. | enantiomer | R3 |
| 104510461047 | rac+− | 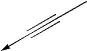 |
| 104810491050 | rac+− | 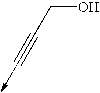 |
| 105110521053 | rac+− | 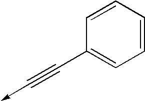 |
| 105410551056 | rac+− | 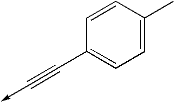 |
| 105710581059 | rac+− | 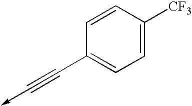 |
| 106010611062 | rac+− | 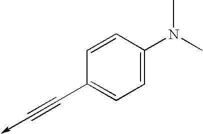 |
| 106310641065 | rac+− | 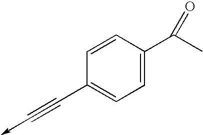 |
| 106610671068 | rac+− | 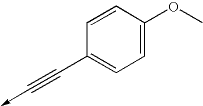 |
| 106910701071 | rac+− | 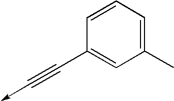 |
| 107210731074 | rac+− | 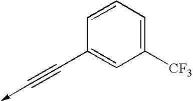 |
| 107510761077 | rac+− | 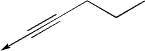 |
| 107810791080 | rac+− | 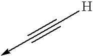 |
| 108110821083 | rac+− | 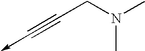 |
| 108410851086 | rac+− | 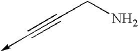 |
| 108710881089 | rac+− | 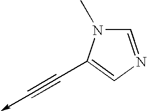 |
| 109010911092 | rac+− | 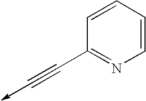 |
| 109310941095 | rac+− | 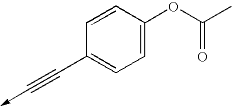 |
| 109610971098 | rac+− | 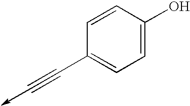 |
| 109911001101 | rac+− | 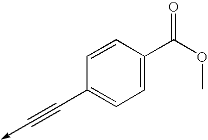 |
| 110211031104 | rac+− | 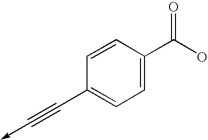 |
| 110511061107 | rac+− | 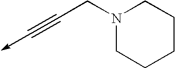 |
| 110811091110 | rac+− | 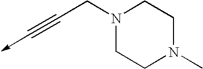 |
| 111111121113 | rac+− | 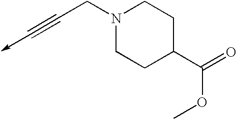 |
| 111411151116 | rac+− | 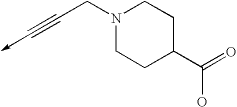 |
| 111711181119 | rac+− | 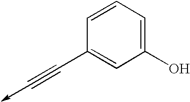 |
| 112011211122 | rac+− | 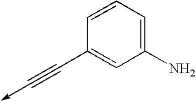 |
| 112311241125 | rac+− | 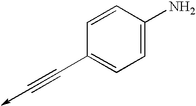 |
| 112611271128 | rac+− | 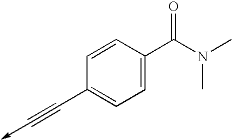 |
| 112911301131 | rac+− | 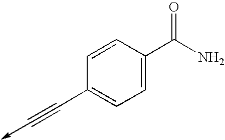 |

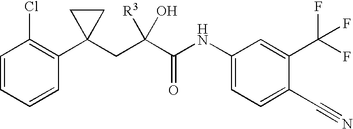

| | racemic or | |
|---|---|---|
| No. | enantiomer | R3 |
| 113211331134 | rac+− | 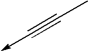 |
| 113511361137 | rac+− | 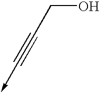 |
| 113811391140 | rac+− | 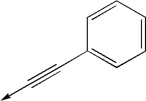 |
| 114111421143 | rac+− | 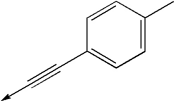 |
| 114411451146 | rac+− | 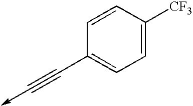 |
| 114711481149 | rac+− | 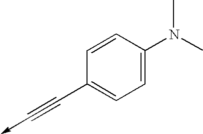 |
| 115011511152 | rac+− | 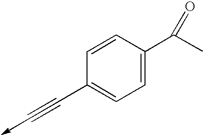 |
| 115311541155 | rac+− | 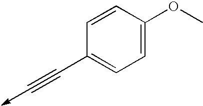 |
| 115611571158 | rac+− | 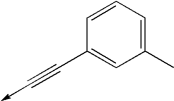 |
| 115911601161 | rac+− | 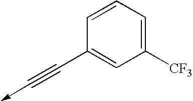 |
| 116211631164 | rac+− | 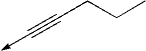 |
| 116511661167 | rac+− | 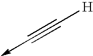 |
| 116811691170 | rac+− | 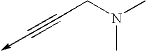 |
| 117111721173 | rac+− | 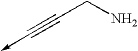 |
| 117411751176 | rac+− | 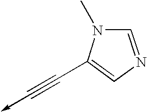 |
| 117711781179 | rac+− | 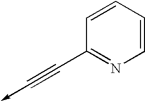 |
| 118011811182 | rac+− | 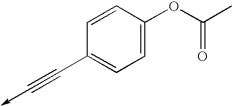 |
| 118311841185 | rac+− | 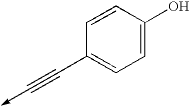 |
| 118611871188 | rac+− | 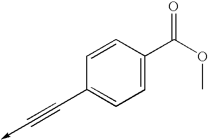 |
| 118911901191 | rac+− | 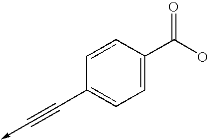 |
| 119211931194 | rac+− | 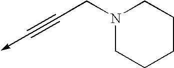 |
| 119511961197 | rac+− | 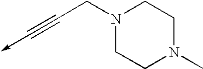 |
| 119811991200 | rac+− | 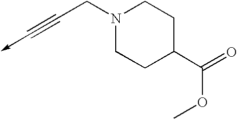 |
| 120112021203 | rac+− | 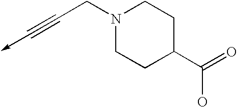 |
| 120412051206 | rac+− | 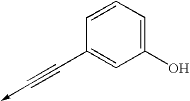 |
| 120712081209 | rac+− | 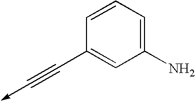 |
| 121012111212 | rac+− | 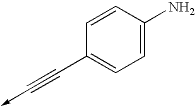 |
| 121312141215 | rac+− | 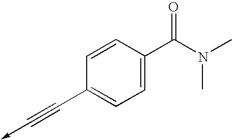 |
| 121612171218 | rac+− | 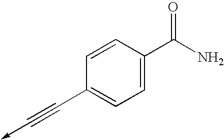 |

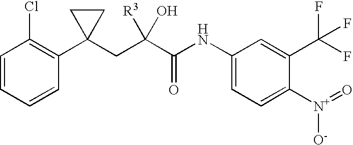

| | racemic or | |
|---|---|---|
| No. | enantiomer | R3 |
| 121912201221 | rac+− | 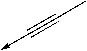 |
| 122212231224 | rac+− | 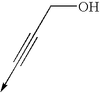 |
| 122512261227 | rac+− | 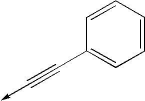 |
| 122812291230 | rac+− | 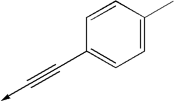 |
| 123112321233 | rac+− | 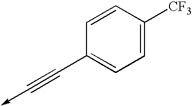 |
| 123412351236 | rac+− | 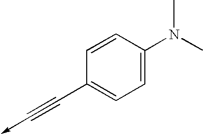 |
| 123712381239 | rac+− | 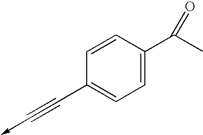 |
| 124012411242 | rac+− | 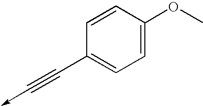 |
| 124312441245 | rac+− | 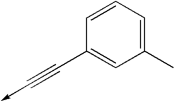 |
| 124612471248 | rac+− | 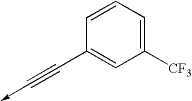 |
| 124912501251 | rac+− | 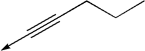 |
| 125212531254 | rac+− | 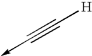 |
| 125512561257 | rac+− | 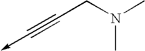 |
| 125812591260 | rac+− | 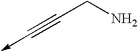 |
| 126112621263 | rac+− | 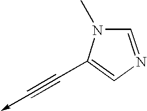 |
| 126412651266 | rac+− | 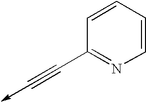 |
| 126712681269 | rac+− | 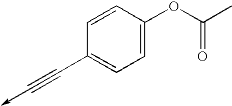 |
| 127012711272 | rac+− | 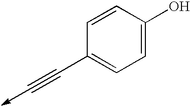 |
| 127312741275 | rac+− | 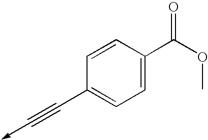 |
| 127612771278 | rac+− | 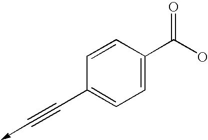 |
| 127912801281 | rac+− | 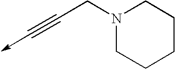 |
| 128212831284 | rac+− | 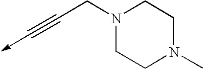 |
| 128512861287 | rac+− | 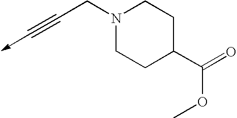 |
| 128812891290 | rac+− | 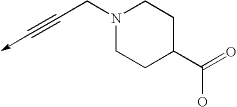 |
| 129112921293 | rac+− | 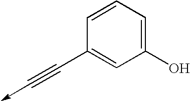 |
| 129412951296 | rac+− | 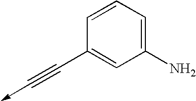 |
| 129712981299 | rac+− | 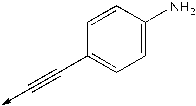 |
| 130013011302 | rac+− | 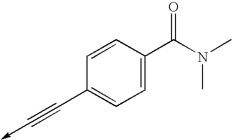 |
| 130313041305 | rac+− | 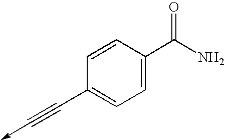 |

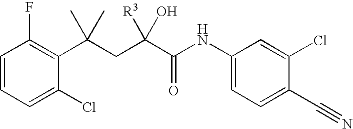

| | racemic or | |
|---|---|---|
| No. | enantiomer | R3 |
| 130613071308 | rac+− | 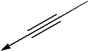 |
| 130913101311 | rac+− | 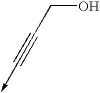 |
| 131213131314 | rac+− | 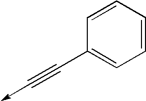 |
| 131513161317 | rac+− | 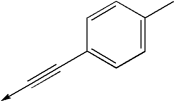 |
| 131813191320 | rac+− | 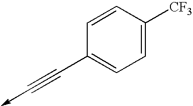 |
| 132113221323 | rac+− | 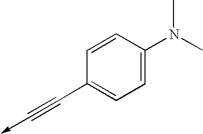 |
| 132413251326 | rac+− | 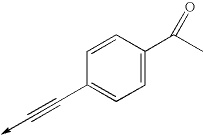 |
| 132713281329 | rac+− | 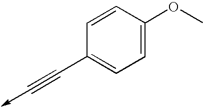 |
| 133013311332 | rac+− | 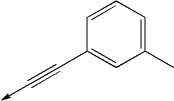 |
| 133313341335 | rac+− | 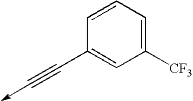 |
| 133613371338 | rac+− | 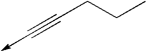 |
| 133913401341 | rac+− | 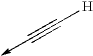 |
| 134213431344 | rac+− | 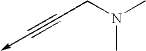 |
| 134513461347 | rac+− | 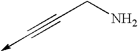 |
| 134813491350 | rac+− | 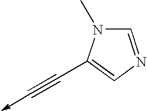 |
| 135113521353 | rac+− | 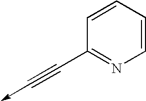 |
| 135413551356 | rac+− | 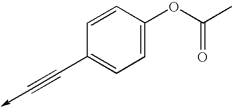 |
| 135713581359 | rac+− | 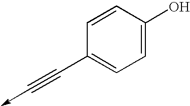 |
| 136013611362 | rac+− | 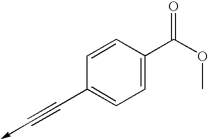 |
| 136313641365 | rac+− | 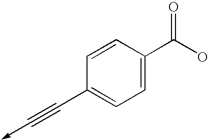 |
| 136613671368 | rac+− | 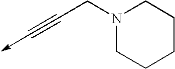 |
| 136913701371 | rac+− | 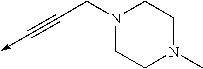 |
| 137213731374 | rac+− | 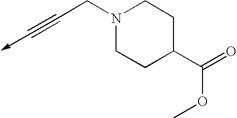 |
| 137513761377 | rac+− | 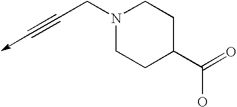 |
| 137813791380 | rac+− | 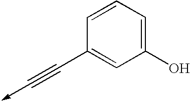 |
| 138113821383 | rac+− | 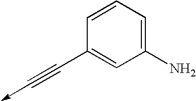 |
| 138413851386 | rac+− | 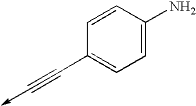 |
| 138713881389 | rac+− | 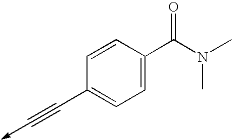 |
| 139013911392 | rac+− | 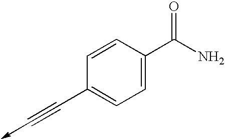 |

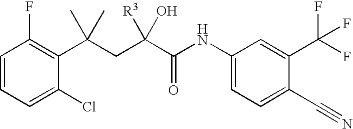

| | racemic or | |
|---|---|---|
| No. | enantiomer | R3 |
| 139313941395 | rac+− | 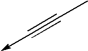 |
| 139613971398 | rac+− | 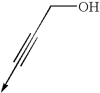 |
| 139914001401 | rac+− | 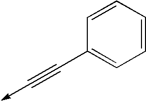 |
| 140214031404 | rac+− | 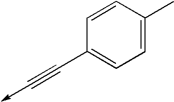 |
| 140514061407 | rac+− | 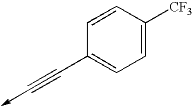 |
| 140814091410 | rac+− | 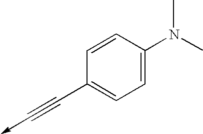 |
| 141114121413 | rac+− | 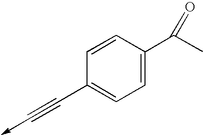 |
| 141414151416 | rac+− | 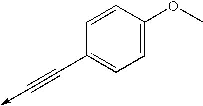 |
| 141714181419 | rac+− | 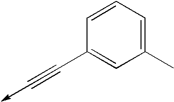 |
| 142014211422 | rac+− | 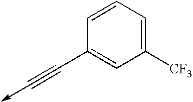 |
| 142314241425 | rac+− | 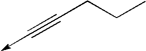 |
| 142614271428 | rac+− | 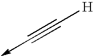 |
| 142914301431 | rac+− | 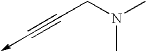 |
| 143214331434 | rac+− | 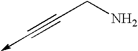 |
| 143514361437 | rac+− | 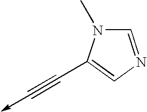 |
| 143814391440 | rac+− | 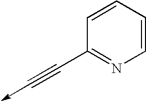 |
| 144114421443 | rac+− | 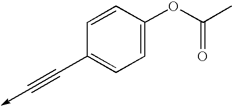 |
| 144414451446 | rac+− | 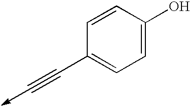 |
| 144714481449 | rac+− | 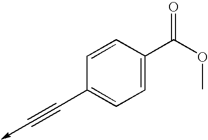 |
| 145014511452 | rac+− | 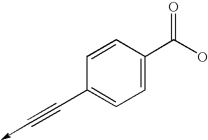 |
| 145314541455 | rac+− | 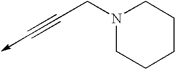 |
| 145614571458 | rac+− | 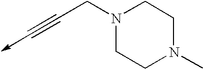 |
| 145914601461 | rac+− | 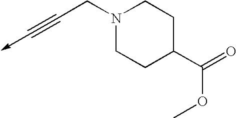 |
| 146214631464 | rac+− | 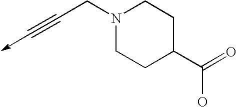 |
| 146514661467 | rac+− | 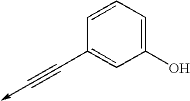 |
| 146814691470 | rac+− | 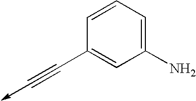 |
| 147114721473 | rac+− | 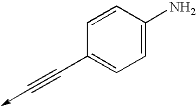 |
| 147414751476 | rac+− | 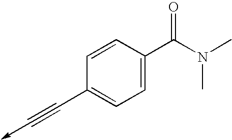 |
| 147714781479 | rac+− | 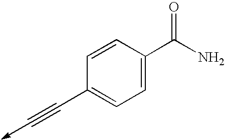 |

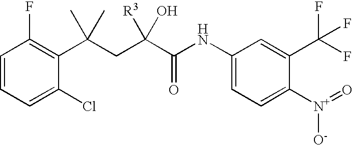

| | racemic or | |
|---|---|---|
| No. | enantiomer | R3 |
| 148014811482 | rac+− | 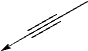 |
| 148314841485 | rac+− | 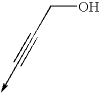 |
| 148614871488 | rac+− | 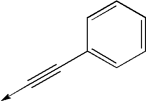 |
| 148914901491 | rac+− | 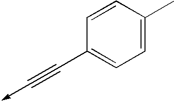 |
| 149214931494 | rac+− | 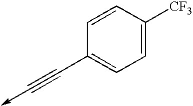 |
| 149514961497 | rac+− | 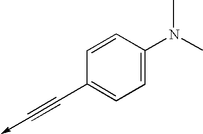 |
| 149814991500 | rac+− | 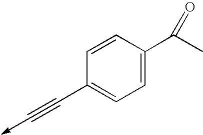 |
| 150115021503 | rac+− | 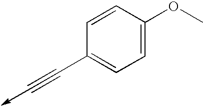 |
| 150415051506 | rac+− | 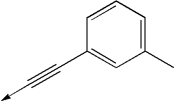 |
| 150715081509 | rac+− | 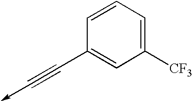 |
| 151015111512 | rac+− | 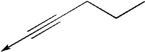 |
| 151315141515 | rac+− | 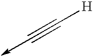 |
| 151615171518 | rac+− | 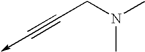 |
| 151915201521 | rac+− | 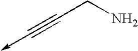 |
| 152215231524 | rac+− | 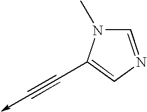 |
| 152515261527 | rac+− | 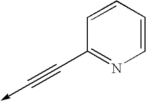 |
| 152815291530 | rac+− | 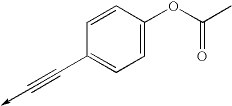 |
| 153115321533 | rac+− | 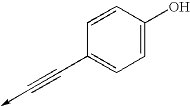 |
| 153415351536 | rac+− | 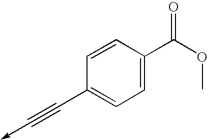 |
| 153715381539 | rac+− | 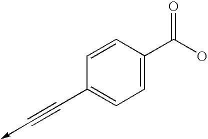 |
| 154015411542 | rac+− | 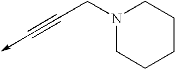 |
| 154315441545 | rac+− | 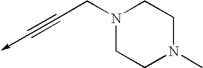 |
| 154615471548 | rac+− | 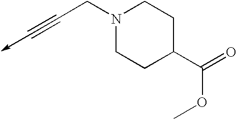 |
| 154915501551 | rac+− | 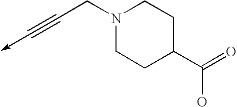 |
| 155215531554 | rac+− | 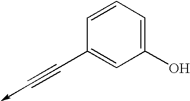 |
| 155515561557 | rac+− | 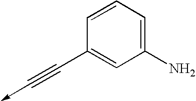 |
| 155815591560 | rac+− | 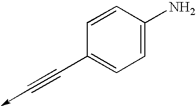 |
| 156115621563 | rac+− | 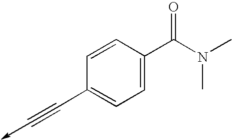 |
| 156415651566 | rac+− | 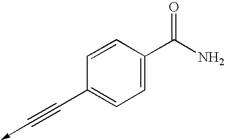 |

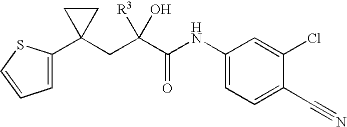

| | racemic or | |
|---|---|---|
| No. | enantiomer | R3 |
| 156715681569 | rac+− | 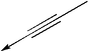 |
| 157015711572 | rac+− | 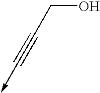 |
| 157315741575 | rac+− | 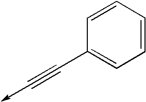 |
| 157615771578 | rac+− | 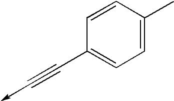 |
| 157915801581 | rac+− | 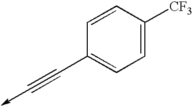 |
| 158215831584 | rac+− | 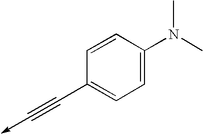 |
| 158515861587 | rac+− | 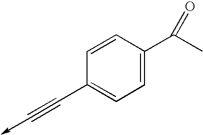 |
| 158815891590 | rac+− | 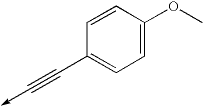 |
| 159115921593 | rac+− | 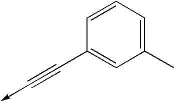 |
| 159415951596 | rac+− | 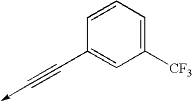 |
| 159715981599 | rac+− | 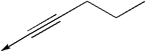 |
| 160016011602 | rac+− | 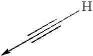 |
| 160316041605 | rac+− | 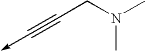 |
| 160616071608 | rac+− | 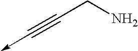 |
| 160916101611 | rac+− | 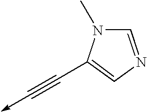 |
| 161216131614 | rac+− | 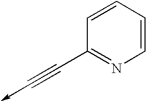 |
| 161516161617 | rac+− | 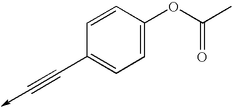 |
| 161816191620 | rac+− | 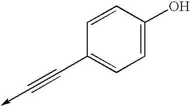 |
| 162116221623 | rac+− | 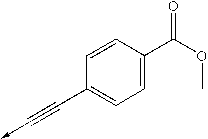 |
| 162416251626 | rac+− | 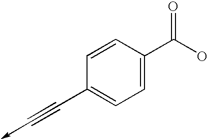 |
| 162716281629 | rac+− | 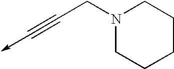 |
| 163016311632 | rac+− | 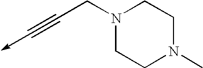 |
| 163316341635 | rac+− | 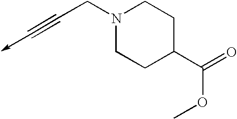 |
| 163616371638 | rac+− | 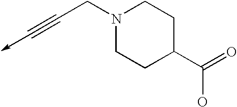 |
| 163916401641 | rac+− | 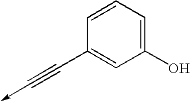 |
| 164216431644 | rac+− | 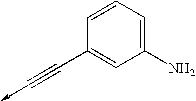 |
| 164516461647 | rac+− | 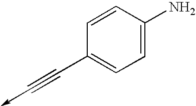 |
| 164816491650 | rac+− | 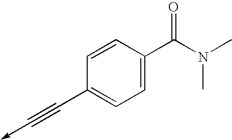 |
| 165116521653 | rac+− | 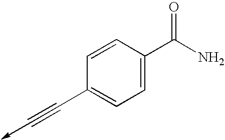 |

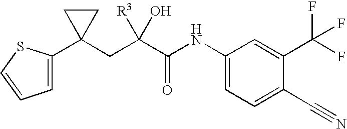

| | racemic or | |
|---|---|---|
| No. | enantiomer | R3 |
| 165416551656 | rac+− | 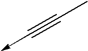 |
| 165716581659 | rac+− | 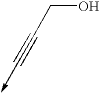 |
| 166016611662 | rac+− | 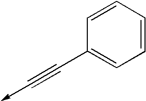 |
| 166316641665 | rac+− | 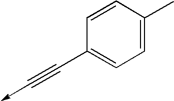 |
| 166616671668 | rac+− | 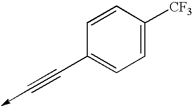 |
| 166916701671 | rac+− | 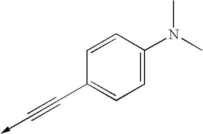 |
| 167216731674 | rac+− | 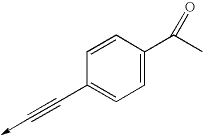 |
| 167516761677 | rac+− | 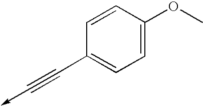 |
| 167816791680 | rac+− | 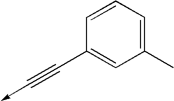 |
| 168116821683 | rac+− | 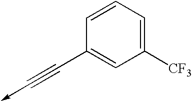 |
| 168416851686 | rac+− | 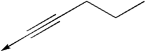 |
| 168716881689 | rac+− | 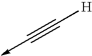 |
| 169016911692 | rac+− | 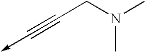 |
| 169316941695 | rac+− | 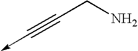 |
| 169616971698 | rac+− | 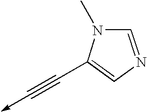 |
| 169917001701 | rac+− | 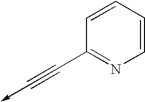 |
| 170217031704 | rac+− | 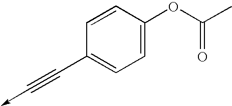 |
| 170517061707 | rac+− | 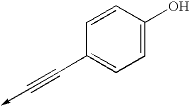 |
| 170817091710 | rac+− | 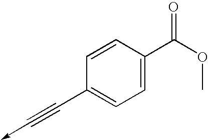 |
| 171117121713 | rac+− | 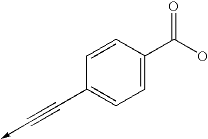 |
| 171417151716 | rac+− | 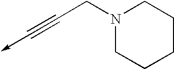 |
| 171717181719 | rac+− | 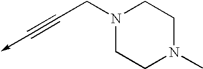 |
| 172017211722 | rac+− | 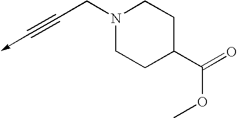 |
| 172317241725 | rac+− | 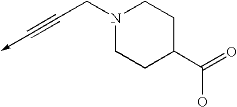 |
| 172617271728 | rac+− | 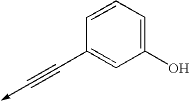 |
| 172917301731 | rac+− | 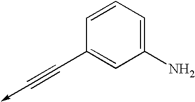 |
| 173217331734 | rac+− | 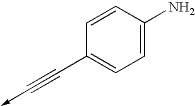 |
| 173517361737 | rac+− | 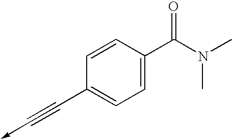 |
| 173817391740 | rac+− | 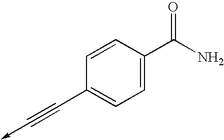 |

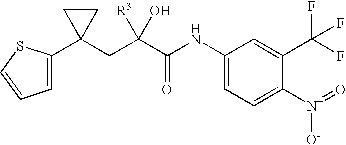

| | racemic or | |
|---|---|---|
| No. | enantiomer | R3 |
| 174117421743 | rac+− | 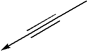 |
| 174417451746 | rac+− | 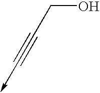 |
| 174717481749 | rac+− | 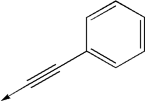 |
| 175017511752 | rac+− | 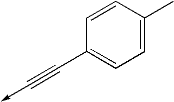 |
| 175317541755 | rac+− | 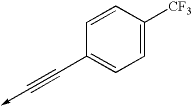 |
| 175617571758 | rac+− | 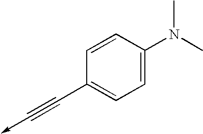 |
| 175917601761 | rac+− | 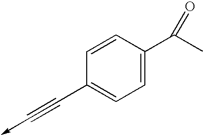 |
| 176217631764 | rac+− | 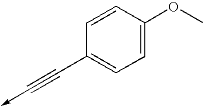 |
| 176517661767 | rac+− | 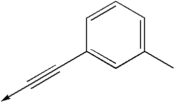 |
| 176817691770 | rac+− | 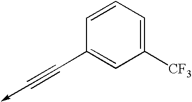 |
| 177117721773 | rac+− | 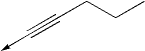 |
| 177417751776 | rac+− | 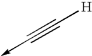 |
| 177717781779 | rac+− | 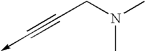 |
| 178017811782 | rac+− | 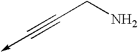 |
| 178317841785 | rac+− | 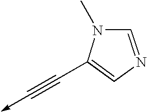 |
| 178617871788 | rac+− | 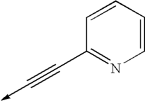 |
| 178917901791 | rac+− | 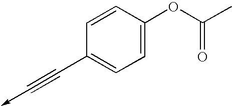 |
| 179217931794 | rac+− | 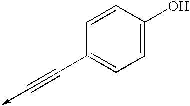 |
| 179517961797 | rac+− | 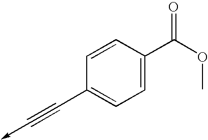 |
| 179817991800 | rac+− | 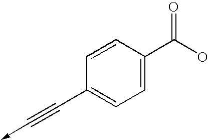 |
| 180118021803 | rac+− | 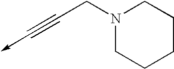 |
| 180418051806 | rac+− | 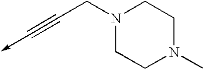 |
| 180718081809 | rac+− | 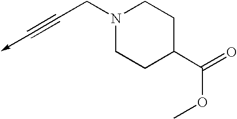 |
| 181018111812 | rac+− | 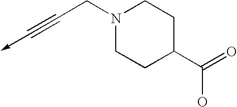 |
| 181318141815 | rac+− | 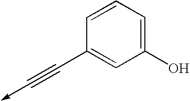 |
| 181618171818 | rac+− | 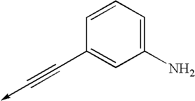 |
| 181918201821 | rac+− | 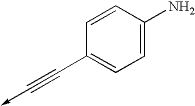 |
| 182218231824 | rac+− | 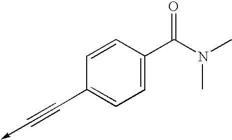 |
| 182518261827 | rac+− | 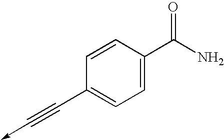 |

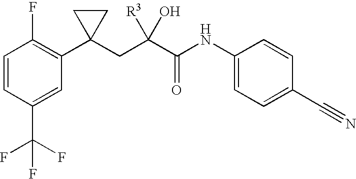

| | racemic or | |
|---|---|---|
| No. | enantiomer | R3 |
| 182818291830 | rac+− | 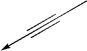 |
| 183118321833 | rac+− | 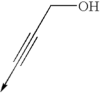 |
| 183418351836 | rac+− | 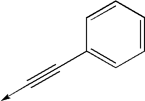 |
| 183718381839 | rac+− | 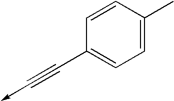 |
| 184018411842 | rac+− | 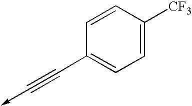 |
| 184318441845 | rac+− | 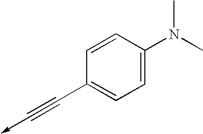 |
| 184618471848 | rac+− | 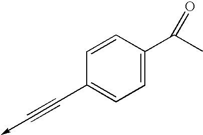 |
| 184918501851 | rac+− | 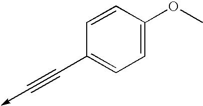 |
| 185218531854 | rac+− | 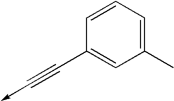 |
| 185518561857 | rac+− | 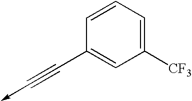 |
| 185818591860 | rac+− | 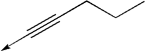 |
| 186118621863 | rac+− | 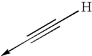 |
| 186418651866 | rac+− | 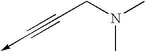 |
| 186718681869 | rac+− | 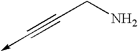 |
| 187018711872 | rac+− | 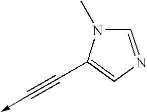 |
| 187318741875 | rac+− | 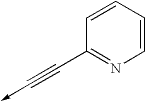 |
| 187618771878 | rac+− | 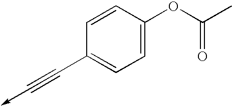 |
| 187918801881 | rac+− | 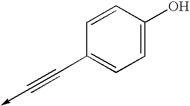 |
| 188218831884 | rac+− | 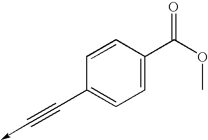 |
| 188518861887 | rac+− | 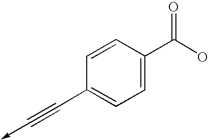 |
| 188818891890 | rac+− | 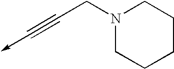 |
| 189118921893 | rac+− | 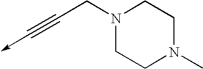 |
| 189418951896 | rac+− | 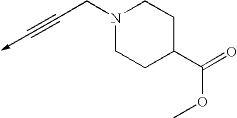 |
| 189718981899 | rac+− | 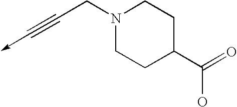 |
| 190019011902 | rac+− | 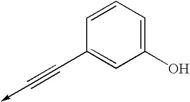 |
| 190319041905 | rac+− | 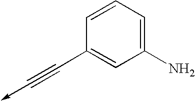 |
| 190619071908 | rac+− | 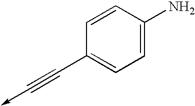 |
| 190919101911 | rac+− | 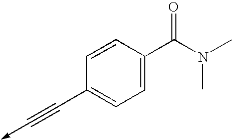 |
| 191219131914 | rac+− | 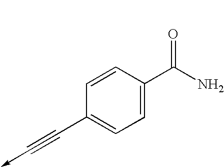 |

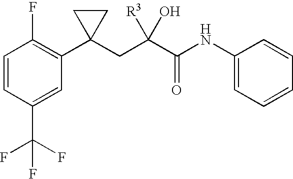

| | racemic or | |
|---|---|---|
| No. | enantiomer | R3 |
| 191519161917 | rac+− | 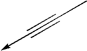 |
| 191819191920 | rac+− | 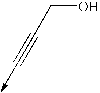 |
| 192119221923 | rac+− | 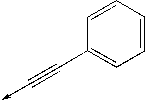 |
| 192419251926 | rac+− | 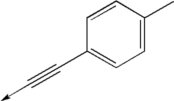 |
| 192719281929 | rac+− | 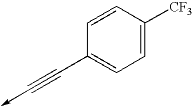 |
| 193019311932 | rac+− | 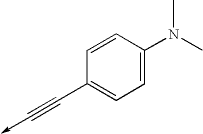 |
| 193319341935 | rac+− | 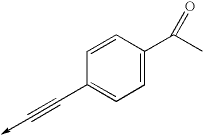 |
| 193619371938 | rac+− | 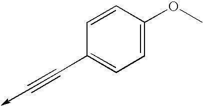 |
| 193919401941 | rac+− | 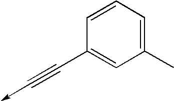 |
| 194219431944 | rac+− | 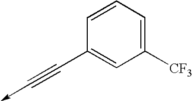 |
| 194519461947 | rac+− | 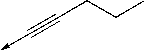 |
| 194819491950 | rac+− | 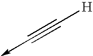 |
| 195119521953 | rac+− | 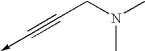 |
| 195419551956 | rac+− | 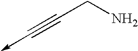 |
| 195719581959 | rac+− | 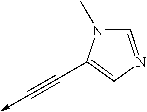 |
| 196019611962 | rac+− | 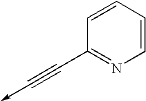 |
| 196319641965 | rac+− | 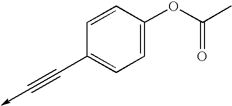 |
| 196619671968 | rac+− | 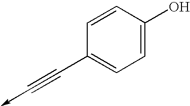 |
| 196919701971 | rac+− | 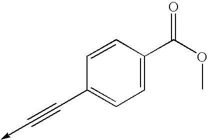 |
| 197219731974 | rac+− | 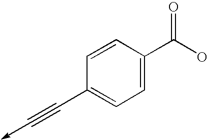 |
| 197519761977 | rac+− | 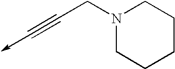 |
| 197819791980 | rac+− | 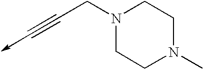 |
| 198119821983 | rac+− | 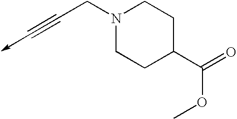 |
| 198419851986 | rac+− | 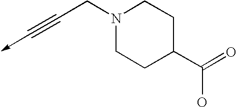 |
| 198719881989 | rac+− | 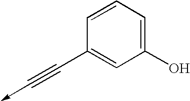 |
| 199019911992 | rac+− | 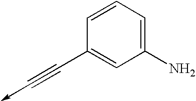 |
| 199319941995 | rac+− | 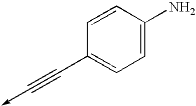 |
| 199619971998 | rac+− | 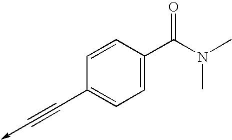 |
| 199920002001 | rac+− | 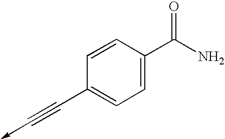 |

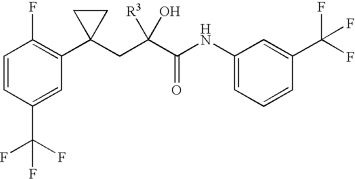

| | racemic or | |
|---|---|---|
| No. | enantiomer | R3 |
| 200220032004 | rac+− | 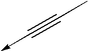 |
| 200520062007 | rac+− | 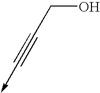 |
| 200820092010 | rac+− | 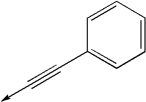 |
| 201120122013 | rac+− | 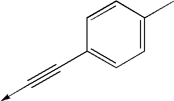 |
| 201420152016 | rac+− | 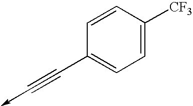 |
| 201720182019 | rac+− | 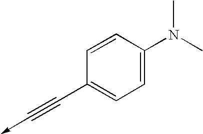 |
| 202320212022 | rac+− | 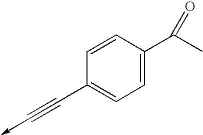 |
| 202320242025 | rac+− | 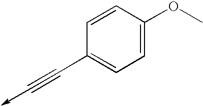 |
| 202620272028 | rac+− | 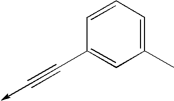 |
| 202920302031 | rac+− | 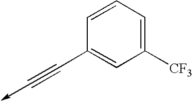 |
| 203220332034 | rac+− | 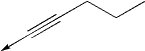 |
| 203520362037 | rac+− | 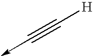 |
| 203820392040 | rac+− | 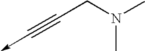 |
| 204120422043 | rac+− | 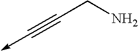 |
| 204420452046 | rac+− | 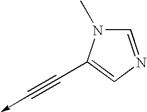 |
| 204720482049 | rac+− | 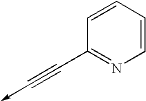 |
| 205020512052 | rac+− | 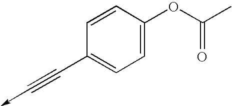 |
| 205320542055 | rac+− | 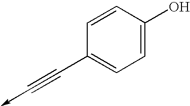 |
| 205620572058 | rac+− | 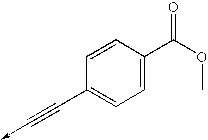 |
| 205920602061 | rac+− | 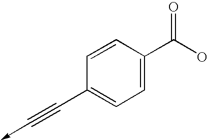 |
| 206220632064 | rac+− | 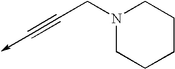 |
| 206520662067 | rac+− | 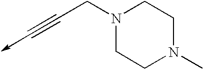 |
| 206820692070 | rac+− | 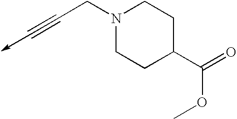 |
| 207120722073 | rac+− | 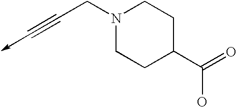 |
| 207420752076 | rac+− | 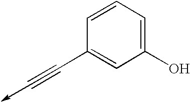 |
| 207720782079 | rac+− | 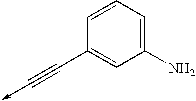 |
| 208020812082 | rac+− | 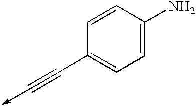 |
| 208320842085 | rac+− | 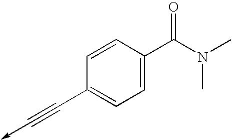 |
| 208620872088 | rac+− | 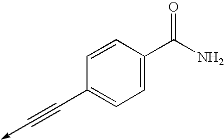 |

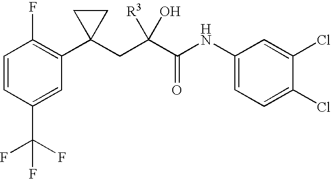

| | racemic or | |
|---|---|---|
| No. | enantiomer | R3 |
| 208920902091 | rac+− | 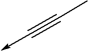 |
| 209220932094 | rac+− | 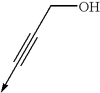 |
| 209520962097 | rac+− | 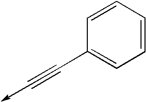 |
| 209820992100 | rac+− | 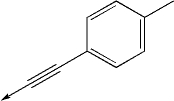 |
| 210121022103 | rac+− | 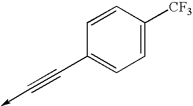 |
| 210421052106 | rac+− | 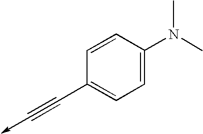 |
| 210721082109 | rac+− | 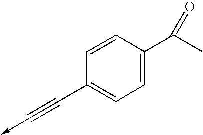 |
| 211021112112 | rac+− | 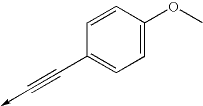 |
| 211321142115 | rac+− | 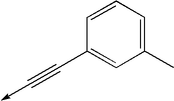 |
| 211621172118 | rac+− | 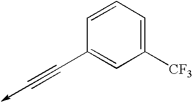 |
| 211921202121 | rac+− | 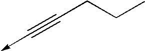 |
| 212221232124 | rac+− | 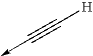 |
| 212521262127 | rac+− | 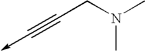 |
| 212821292130 | rac+− | 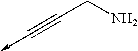 |
| 213121322133 | rac+− | 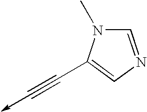 |
| 213421352136 | rac+− | 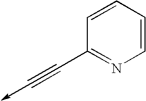 |
| 213721382139 | rac+− | 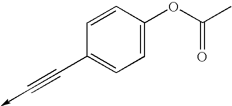 |
| 214021412142 | rac+− | 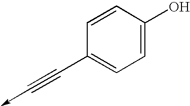 |
| 214321442145 | rac+− | 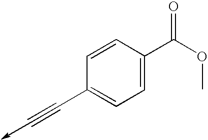 |
| 214621472148 | rac+− | 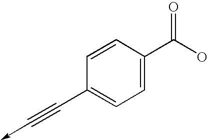 |
| 214921502151 | rac+− | 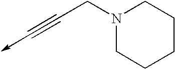 |
| 215221532154 | rac+− | 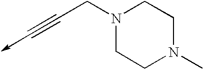 |
| 215521562157 | rac+− | 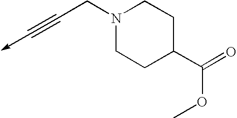 |
| 215821592160 | rac+− | 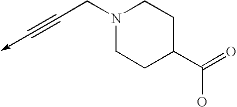 |
| 216121622163 | rac+− | 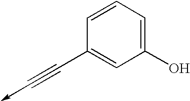 |
| 216421652166 | rac+− | 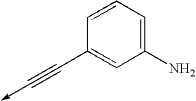 |
| 216721682169 | rac+− | 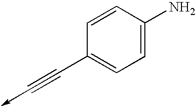 |
| 217021712172 | rac+− | 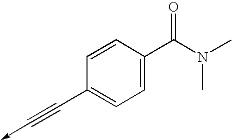 |
| 217321742175 | rac+− | 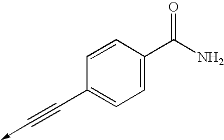 |

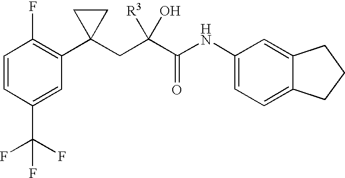

| | racemic or | |
|---|---|---|
| No. | enantiomer | R3 |
| 217621772178 | rac+− | 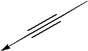 |
| 217921802181 | rac+− | 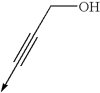 |
| 218221832184 | rac+− | 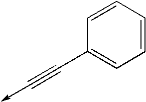 |
| 218521862187 | rac+− | 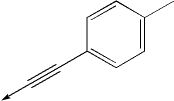 |
| 218821892190 | rac+− | 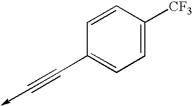 |
| 219121922193 | rac+− | 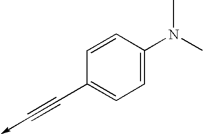 |
| 219421952196 | rac+− | 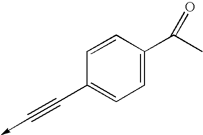 |
| 219721982199 | rac+− | 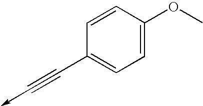 |
| 220022012202 | rac+− | 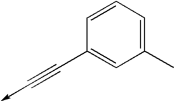 |
| 220322042205 | rac+− | 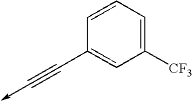 |
| 220622072208 | rac+− | 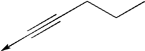 |
| 220922102211 | rac+− | 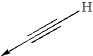 |
| 221222132214 | rac+− | 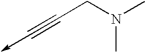 |
| 221522162217 | rac+− | 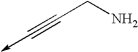 |
| 221822192220 | rac+− | 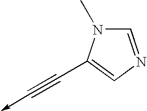 |
| 222122222223 | rac+− | 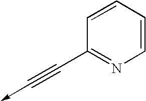 |
| 222422252226 | rac+− | 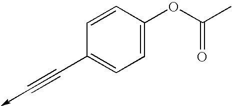 |
| 222722282229 | rac+− | 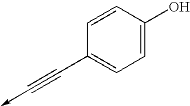 |
| 223022312232 | rac+− | 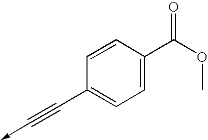 |
| 223322342235 | rac+− | 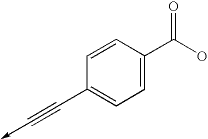 |
| 223622372238 | rac+− | 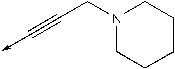 |
| 223922402241 | rac+− | 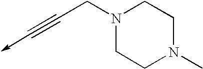 |
| 224222432244 | rac+− | 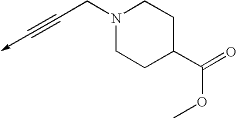 |
| 224522462247 | rac+− | 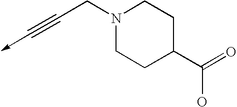 |
| 224822492250 | rac+− | 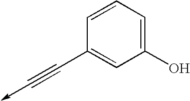 |
| 225122522253 | rac+− | 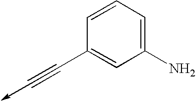 |
| 225422552256 | rac+− | 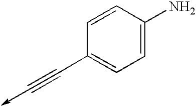 |
| 225722582259 | rac+− | 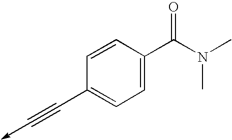 |
| 226022612262 | rac+− | 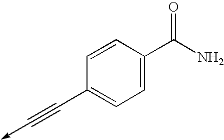 |

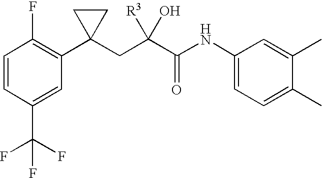

| | racemic or | |
|---|---|---|
| No. | enantiomer | R3 |
| 226322642265 | rac+− | 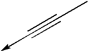 |
| 226622672268 | rac+− | 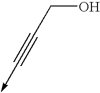 |
| 226922702271 | rac+− | 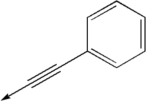 |
| 227222732274 | rac+− | 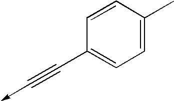 |
| 227522762277 | rac+− | 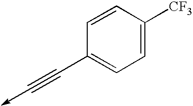 |
| 227822792280 | rac+− | 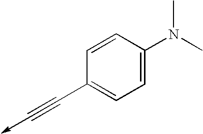 |
| 228122822283 | rac+− | 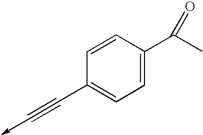 |
| 228422852286 | rac+− | 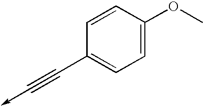 |
| 228722882289 | rac+− | 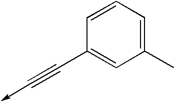 |
| 229022912292 | rac+− | 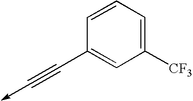 |
| 229322942295 | rac+− | 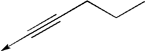 |
| 229622972298 | rac+− | 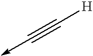 |
| 229923002301 | rac+− | 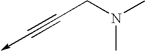 |
| 230223032304 | rac+− | 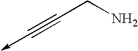 |
| 230523062307 | rac+− | 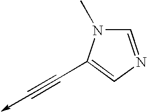 |
| 230823092310 | rac+− | 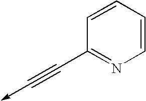 |
| 231123122313 | rac+− | 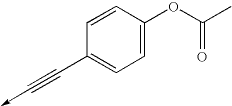 |
| 231423152316 | rac+− | 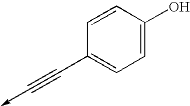 |
| 231723182319 | rac+− | 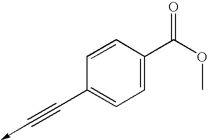 |
| 232023212322 | rac+− | 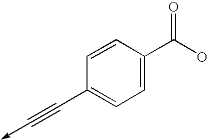 |
| 232323242325 | rac+− | 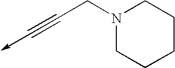 |
| 232623272328 | rac+− | 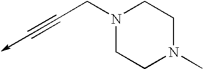 |
| 232923302331 | rac+− | 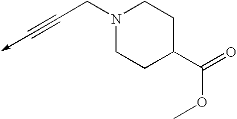 |
| 233223332334 | rac+− | 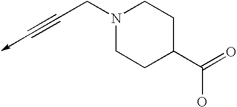 |
| 233523362337 | rac+− | 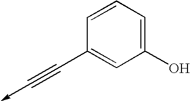 |
| 233823392340 | rac+− | 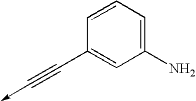 |
| 234123422343 | rac+− | 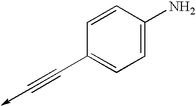 |
| 234423452346 | rac+− | 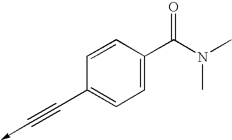 |
| 234723482349 | rac+− | 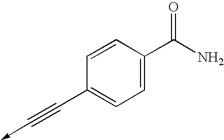 |

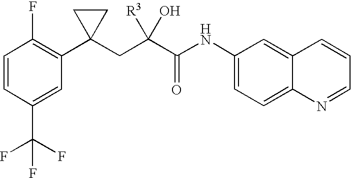

| | racemic or | |
|---|---|---|
| No. | enantiomer | R3 |
| 235023512352 | rac+− | 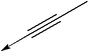 |
| 235323542355 | rac+− | 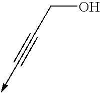 |
| 235623572358 | rac+− | 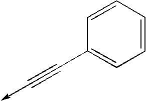 |
| 235923602361 | rac+− | 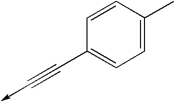 |
| 236223632364 | rac+− | 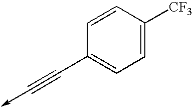 |
| 236523662367 | rac+− | 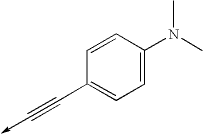 |
| 236823692370 | rac+− | 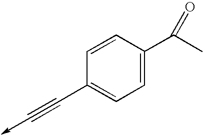 |
| 237123722373 | rac+− | 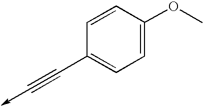 |
| 237423752376 | rac+− | 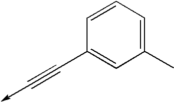 |
| 237723782379 | rac+− | 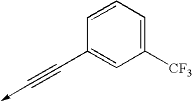 |
| 238023812382 | rac+− | 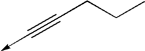 |
| 238323842385 | rac+− | 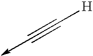 |
| 238623872388 | rac+− | 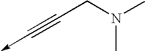 |
| 238923902391 | rac+− | 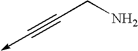 |
| 239223932394 | rac+− | 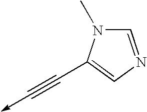 |
| 239523962397 | rac+− | 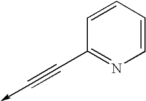 |
| 239823992400 | rac+− | 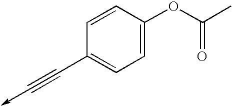 |
| 240124022403 | rac+− | 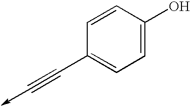 |
| 240424052406 | rac+− | 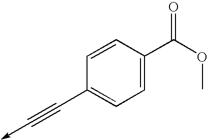 |
| 240724082409 | rac+− | 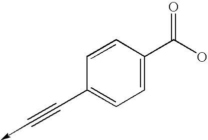 |
| 241024112412 | rac+− | 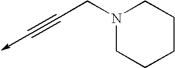 |
| 241324142415 | rac+− | 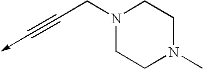 |
| 241624172418 | rac+− | 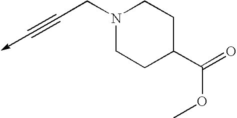 |
| 241924202421 | rac+− | 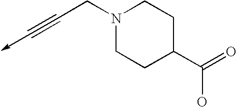 |
| 242224232424 | rac+− | 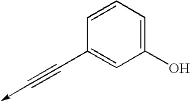 |
| 242524262427 | rac+− | 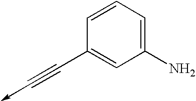 |
| 242824292430 | rac+− | 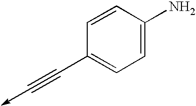 |
| 243124322433 | rac+− | 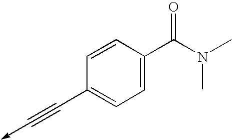 |
| 243424352436 | rac+− | 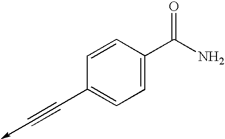 |

### Biological Characterization of the Compounds According to the Invention

Progesterone receptor modulators can be identified with the aid of simple methods, test programmes known to the skilled person. It is possible for this purpose for example to incubate a compound to be tested together with a progestogen in a test system for progesterone receptor ligands and to check whether the effect mediated by progesterone is altered in the presence of the modulator in this test system.

The substances according to the invention of the general formula I were tested in the following models:

### Progesterone Receptor-Binding Assay

Measurement of the receptor binding affinity:

The receptor binding affinity was determined by competitive binding of a specifically binding ³H-labelled hormone (tracer) and of the compound to be tested on receptors in the cytosol from animal target organs. The aim in this case was receptor saturation and reaction equilibrium.

The tracer and increasing concentrations of the compound to be tested (competitor) were coincubated at 0-4° C. for 18 h with the receptor-containing cytosol fraction. After removal of unbound tracer with carbon-dextran suspension, the receptor-bound tracer content was measured for each concentration, and the IC₅₀ was determined from the concentration series. The relative molar binding affinity (RBA) was calculated as ratio of the IC₅₀ values for reference substance and compound to be tested (×100%) (RBA of the reference substance=100%).

The following incubation conditions were chosen for the receptor types:

### Progesterone Receptor:

Uterus cytosol of the estradiol-primed rabbit, homogenized in TED buffer (20 mMTris/HCl, pH 7.4; 1 mM ethylenediaminetetraacetate, 2 mM dithiothreitol) with 250 mM sucrose; stored at −30° C. Tracer: ³H-ORG 2058, 5 nM; reference substance: progesterone.

### Glucocorticoid Receptor:

Thymus cytosol from the adrenalectomized rat, thymi stored at −30° C.; buffer: TED. Tracer: ³H-dexamethasone, 20 nM; reference substance: dexamethasone.

The competition factors (CF values) for the compounds according to the invention of the general formula (I) on the progesterone receptor are between 0.2 and 35 relative to progesterone. The CF values on the glucocorticoid receptor are in the range from 3 to 35 relative to dexamethasone.

The compounds according to the invention accordingly have a high affinity for the progesterone receptor, but only a low affinity for the glucocorticoid receptor.

### Antagonism at the PR Progesterone Receptor

The transactivation assay is carried out as described in WO 02/054064.

The IC₅₀ values are in the range of from 0.1 to 150 nM.

### Agonism on the PR Progesterone Receptor

The transactivation assay is carried out as described in Fuhrmann et al. (Fuhrmann U., Hess-Stump H., Cleve A., Neef G., Schwede W., Hoffmann J., Fritzemeier K.-H., Chwalisz K., Journal of Medicinal Chemistry, 43, 26, 2000, 5010-5016). The EC₅₀ values are in the range from 0.01 to 150 nM.

### Dosage

The progesterone receptor modulators can be administered orally, enterally, parenterally or transdermally for the use according to the invention.

Satisfactory results are generally to be expected in the treatment of the indications mentioned hereinbefore when the daily doses cover a range from 1 μg to 1000 mg of the compound according to the invention for gynaecological indications such as treatment of endometriosis, leiomyomas of the uterus and dysfunctional bleeding, and for use in fertility control and for hormone replacement therapy. Daily dosages to be administered for oncological indications are in the range from 1 μg to 2000 mg of the compound according to the invention.

Suitable dosages of the compounds according to the invention in humans for the treatment of endometriosis, of leiomyomas of the uterus and dysfunctional bleeding and for use in fertility control and for hormone replacement therapy are from 50 μg to 500 mg per day, depending on the age and constitution of the patient, it being possible to administer the necessary daily dose by single or multiple administration.

The dosage range for the compounds according to the invention for the treatment of breast carcinomas is 10 mg to 2000 mg per day.

The pharmaceutical products based on the novel compounds are formulated in a manner known per se by processing the active ingredient with the carrier substances, fillers, substances influencing disintegration, binders, humectants, lubricants, absorbents, diluents, masking flavours, colorants, etc. which are used in pharmaceutical technology, and converting into the desired administration form. Reference should be made in this connection to Remington's Pharmaceutical Science, 15^{th} ed. Mack Publishing Company, East Pennsylvania (1980).

Suitable for oral administration are in particular tablets, film-coated tablets, sugar-coated tablets, capsules, pills, powders, granules, pastilles, suspensions, emulsions or solutions.

Preparations for injection and infusion are possible for parenteral administration.

Appropriately prepared crystal suspensions can be used for intraarticular injection.

Aqueous and oily solutions for injection or suspensions and corresponding depot preparations can be used for intramuscular injection.

For rectal administration, the novel compounds can be used in the form of suppositories, capsules, solutions (e.g. in the form of enemas) and ointments, both for systemic and for local therapy.

Furthermore, compositions for vaginal use may also be mentioned as preparation.

For pulmonary administration of the novel compounds, they can be used in the form of aerosols and inhalants.

Patches are possible for transdermal administration, and formulations in gels, ointments, fatty ointments, creams, pastes, dusting powders, milk and tinctures are possible for topical application. The dosage of the compounds of the general formula I in these preparations should be 0.01%-20% in order to achieve an adequate pharmacological effect.

Corresponding tablets can be obtained for example by mixing active ingredient with known excipients, for example inert diluents such as dextrose, sugar, sorbitol, mannitol, polyvinylpyrrolidone, disintegrants such as maize starch or alginic acid, binders such as starch or gelatin, lubricants such as magnesium stearate or talc and/or means to achieve a depot effect such as carboxypolymethylene, carboxymethylcellulose, cellulose acetate phthalate or polyvinyl acetate. The tablets may also consist of a plurality of layers.

Correspondingly, coated tablets can be produced by coating cores produced in analogy to the tablets with compositions normally used in tablet coatings, for example polyvinylpyrrolidone or shellac, gum arabic, talc, titanium oxide or sugar. The tablet covering may in this case also consist of a plurality of layers, it being possible to use the excipients mentioned above for tablets.

Solutions or suspensions of the compounds according to the invention of the general formula I may additionally comprise taste-improving agents such as saccharin, cyclamate or sugar, and, for example, flavourings such as vanillin or orange extract. They may additionally comprise suspending excipients such as sodium carboxymethylcellulose or preservatives such as p-hydroxybenzoates.

Capsules comprising the compounds of the general formula I can be produced for example by mixing the compound(s) of the general formula I with an inert carrier such as lactose or sorbitol and encapsulating it in gelatin capsules.

Suitable suppositories can be produced for example by mixing with carriers intended for this purpose, such as neutral fats or polyethylene glycol or derivatives thereof.

The compounds according to the invention of the general formula (I) or their pharmaceutically acceptable salts can be used, because of their antagonistic or partial agonistic activity, for producing a medicament, in particular for the treatment and prophylaxis of gynaecological disorders such as endometriosis, leiomyomas of the uterus, dysfunctional bleeding and dysmenorrhoea. They can furthermore be employed to counteract hormonal irregularities, for inducing menstruation and alone or in combination with prostaglandins and/or oxytocin to induce labour.

The compounds according to the invention of the general formula (I) or their pharmaceutically acceptable salts are furthermore suitable for producing products for female contraception (see also WO 93/23020, WO 93/21927).

The compounds according to the invention or their pharmaceutically acceptable salts can additionally be employed alone or in combination with estrogens, estrogen derivatives, substances having estrogenic activity or with a selective oestrogen receptor modulator (SERM) for female hormone replacement therapy.

In addition, the said compounds have an antiproliferative effect in hormone-dependent tumours. They are therefore suitable for the therapy of hormone-dependent carcinomas such as, for example, for breast, prostate and endometrial carcinomas.

The compounds according to the invention or their pharmaceutically acceptable salts can be employed for the treatment of hormone-dependent carcinomas both in first-line therapy and in second-line therapy, especially after tamoxifen failure.

The compounds according to the invention, having antagonistic or partially agonistic activity, of the general formula (I) or their pharmaceutically acceptable salts can also be used in combination with compounds having antiestrogenic activity (estrogen receptor antagonists or aromatase inhibitors) or selective estrogen receptor modulators (SERM) for producing pharmaceutical products for the treatment of hormone-dependent tumours. The compounds according to the invention can likewise be used in combination with SERMs or an antiestrogen (estrogen receptor antagonist or aromatase inhibitor) for the treatment of endometriosis or of leiomyomas of the uterus.

Suitable for combination with the non-steroidal progesterone receptor modulators according to the invention in this connection are for example the following antiestrogens (estrogen receptor antagonists or aromatase inhibitors) or SERMs: tamoxifen, 5-(4-{5-[(RS)-(4,4,5,5,5-pentafluoropentyl)sulphynyl]pentyloxy}-phenyl)-6-phenyl-8,9-dihydro-7H-benzocyclohepten-2-ol (WO 00/03979), ICI 182 780 (7alpha-[9-(4,4,5,5-pentafluoropentylsulphynyl)nonyl]estra-1,3,5(10)-triene-3,17-beta-diol), 11beta-fluoro-7alpha-[5-(methyl{3-[(4,4,5,5,5-pentafluoropentyl)sulphanyl]propyl}amino)pentyl]-estra-1,3,5(10)-triene-3,17beta-diol (WO98/07740), 11beta-fluoro-7alpha-{5-[methyl(7,7,8,8,9,9,10,10,10-nonafluorodecyl)amino]pentyl}estra-1,3,5(10)-triene-3,17-beta-diol (WO 99/33855), 11beta-fluoro-17alpha-methyl-7alpha-{5-[methyl(8,8,9,9,9-pentafluorononyl)amino]pentyl}estra-1,3,5(10)-triene-3,17beta-diol (WO 03/045972), clomifene, raloxifene, and further compounds having antiestrogenic activity, and aromatase inhibitors such as, for example, fadrozole, formestane, letrozole, anastrozole or atamestane.

Suitable for combination of the progesterone receptor modulators according to the invention with suitable estrogens, estrogen derivatives or substances having estrogenic activity are the following: 17β-estradiol, 17β-ethinylestradiol, estriol, 17β-estradiol 3-alkylsulphonates, 17β-ethinylestradiol 3-alkylsulphonates, estradiol 3- or 17-esters such as estradiol 3-benzoate or estradiol 17-valerate, 17β-ethinylestradiol 3-ethers such as 17β-ethinylestradiol 3-methyl ether (mestranol) or conjugated equine estrogens (CEE).

In the case of the estrogen 3-alkylsulphonates such as 17β-estradiol 3-alkylsulphonate and 17β-ethinylestradiol 3-alkylsulphonate, suitable for the alkylsulphonate are in particular saturated, branched or unbranched C₁-C₅-alkyl groups, with the meanings mentioned in the definitions on page 9 applying to C₁-C₅-alkyl. Mention may be made here by way of example, without restriction thereto, of 17β-estradiol 3-isopropylsulphonate and of 17β-ethinylestradiol 3-propylsulphonate (turisterone).

Finally, the present invention also relates to the use of the compounds of the general formula I, where appropriate together with an antiestrogen, an estrogen or estrogen derivative and a substance having estrogenic activity, or a SERM, for producing a medicament.

The present invention further relates to pharmaceutical compositions which comprise at least one compound according to the invention, where appropriate in the form of a pharmaceutically/pharmacologically acceptable salt.

These pharmaceutical compositions and medicaments may be intended for oral, rectal, vaginal, subcutaneous, percutaneous, intravenous or intramuscular administration. Besides conventional carriers and/or diluents, they comprise at least one compound according to the invention.

The medicaments of the invention are produced with the conventional solid or liquid carriers or diluents and the excipients normally used in pharmaceutical technology appropriate for the desired mode of administration with a suitable dosage in a known manner. The preferred preparations consist of a dosage suitable for oral administration. Examples of such dosage forms are tablets, film-coated tablets, sugar-coated tablets, capsules, pills, powders, solutions or suspensions or else depot forms.

The pharmaceutical compositions comprising at least one of the compounds according to the invention are preferably administered orally.

Also suitable are parenteral preparations such as solutions for injection. Further preparations which may also be mentioned are for example suppositories and compositions for vaginal use.

### Preparation of the Compounds According to the Invention:

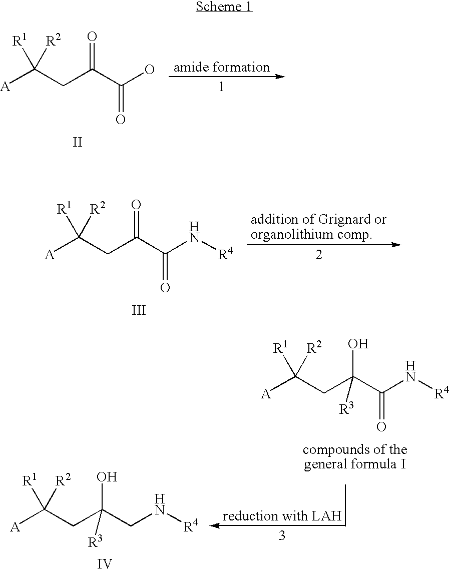

The compounds of the general formula I can be synthesized as shown in scheme 1. Carboxylic acids of the general formula II have been described for example in previously described WO 199854159, WO 200375915 and WO 9854159. The amides of the general formula III are prepared for example by forming the acid chlorides and subsequently reacting with the appropriate amines. However, as an alternative thereto, it is also possible to use other methods for amide formation, depending on the amine to be introduced. The compounds of the general formula I are then prepared from the amides of the general formula III by addition of Grignard or organolithium compounds. Steps 1 and 2 can, however, also be carried out in the reverse sequence.

The substituents A, R¹, R², R³ and R⁴ may also where appropriate be further modified after introduction has taken place. Suitable for this purpose are for example oxidation, reduction, alkylations, acylations, nucleophilic additions or especially also transition metal-catalyzed coupling reactions.

Functional groups in compounds of the general formulae II, III and IV are provided where appropriate with temporary protective groups which are then eliminated again at a suitable stage.

The following examples serve to explain the subject-matter of the invention in more detail, without intending to restrict it thereto.

Preparation of 3-[1-(2-fluoro-5-trifluoromethylphenyl)cyclopropyl]-2-oxopropionic acid is described in WO 200375915.

### EXAMPLE 1

### rac-{2-Hydroxy-3-[1-(2-fluoro-5-trifluoromethylphenyl)cyclopropyl]-2-phenylethynyl)]propionic acid}(3-chloro-4-cyanophenyl)amide

### 1a) {3-[1-(2-Fluoro-5-trifluoromethylphenyl)cyclopropyl]-2-oxopropionic acid}(3-chloro-4-cyanophenyl)amide

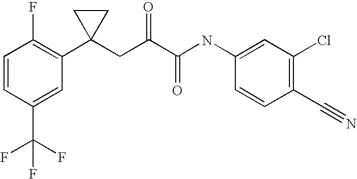

3-[1-(2-Fluoro-5-trifluoromethylphenyl)cyclopropyl]-2-oxopropionic acid (500 mg) was dissolved in 10 ml of N,N-dimethylacetamide. At −10° C., 155 μl of thionyl chloride were added, and the mixture was stirred at −10° C. for one hour. Subsequently, 368 mg of 4-amino-2-chlorobenzonitrile were added in portions. This was followed by stirring for 2 hours (−10° C. to 23° C.). The reaction mixture was then poured into ice-water. It was stirred for 2 hours and filtered with suction. The resulting solid was purified by column chromatography on silica gel with a hexane/ethyl acetate mixture. 495 mg of product were obtained.

¹H-NMR (ppm, CDCl₃, 300 MHz): 1.00 (4H), 3.30 (2H), 7.08 (1H), 7.45-7.57 (2H), 7.60-7.75 (2H), 7.92 (1H), 8.80 (1H).

### 1b) rac-{2-Hydroxy-3-[1-(2-fluoro-5-trifluoromethylphenyl)-cyclopropyl]-2-phenylethynyl)]propionic acid}(3-chloro-4-cyanophenyl)amide

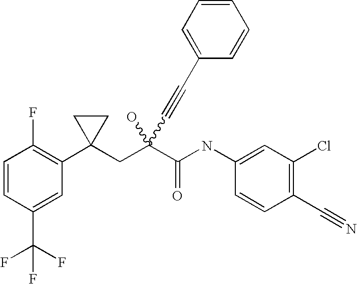

At −78° C., n-butyllithium (314 μl, 1.6 M in hexane) was added to a solution of 62 μl of phenylacetylene in tetrahydrofuran (5 ml). The mixture was stirred at this temperature for 30 minutes and then a solution of the compound (100 mg) described in 1a) in 4 ml of tetrahydrofuran was added dropwise. The mixture was then allowed to reach 23° C. over about 3 h and was subsequently stirred for 10 h. The reaction mixture was then poured into ice-cold saturated ammonium chloride solution. It was extracted with ethyl acetate. The combined organic phases were washed with saturated sodium chloride solution and dried over sodium sulphate. The crude product was chromatographed on silica gel. 93 mg of product were obtained.

¹H-NMR (ppm, CDCl₃, 400 MHz): 0.88 (1H), 0.95-1.11 (3H), 2.41 (1H), 2.66 (1H), 2.99 (1H, 7.02 (1H), 7.22-7.38 (6H), 7.40 (1H), 7.60 (2H), 7.80 (1H), 8.70 (1H).

### EXAMPLE 1c AND 1d

### (+)-{2-Hydroxy-3-[1-(2-fluoro-5-trifluoromethylphenyl)cyclopropyl]-2-phenylethynyl)]propionic acid}(3-chloro-4-cyanophenyl)amide 1c and

### (−)-{2-hydroxy-3-[1-(2-fluoro-5-trifluoromethyl phenyl)cyclopropyl]-2-phenylethynyl)]propionic acid}(3-chloro-4-cyanophenyl)amide 1d

The racemic mixture obtained in Example 1b was separated into the enantiomers 1c and 1d by preparative chiral HPLC (Chiralpak AD 250×10 mm column).

1c: [α]^{D}₂₀=+7.1° (CHCl₃, 8.9 mg/1 ml; λ=589 nM)

1d: [α]^{D}₂₀=−8.7° (CHCl₃, 9.2 mg/l ml; λ=589 nM)

### EXAMPLE 2

### rac-{2-Hydroxy-3-[1-(2-fluoro-5-trifluoromethylphenyl)cyclopropyl]-2-(4-methyl phenyl)ethynyl)]propionic acid}(3-chloro-4-cyanophenyl)amide

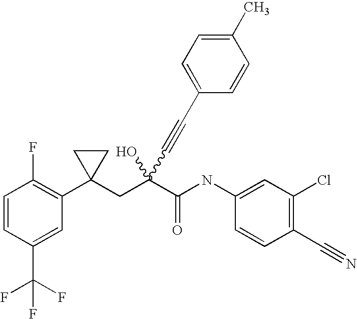

The compound described in Example 2 was prepared from the compound described in 1a), 4-methylphenylacetylene and n-butyllithium in analogy to the process described in Example 1b).

¹H-NMR (ppm, CDCl₃, 300 MHz): 0.86 (1H), 0.92-1.10 (3H), 2.33 (3H), 2.40 (1H), 2.67 (1H), 2.97 (1H), 7.00 (1H), 7.09 (2H), 7.20 (2H), 7.33 (1H), 7.40 (1H), 7.55-7.65 (2H), 7.79 (1H), 8.70 (1H).

### EXAMPLE 3

### rac-{2-Hydroxy-3-[1-(2-fluoro-5-trifluoromethylphenyl)-cyclopropyl]-2-phenylethynyl)]-propionic acid}(4-cyano-3-trifluoromethyl phenyl)amide

### 3a) {3-[1-(2-Fluoro-5-trifluoromethylphenyl)cyclopropyl]-2-oxopropionic acid}(4-cyano-3-trifluoromethylphenyl)amide

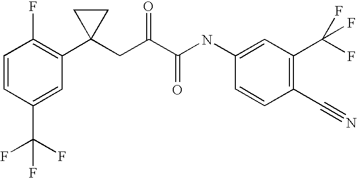

The compound described in Example 3a) was prepared from 3-[1-(2-fluoro-5-trifluoromethylphenyl)cyclopropyl]-2-oxopropionic acid and 4-amino-2-trifluoromethyl-benzonitrile in analogy to the process described in Example 1a).

¹H-NMR (ppm, CDCl₃, 300 MHz): 1.02 (4H), 3.30 (2H), 7.08 (1H), 7.49 (1H), 7.70 (1H), 7.82 (1H), 7.93 (1H), 8.08 (1H), 8.94 (1H).

### 3b) rac-{2-Hydroxy-3-[1-(2-fluoro-5-trifluoromethylphenyl)-cyclopropyl]-2-phenylethynyl)]-propionic acid}(4-cyano-3-trifluoromethylphenyl)amide

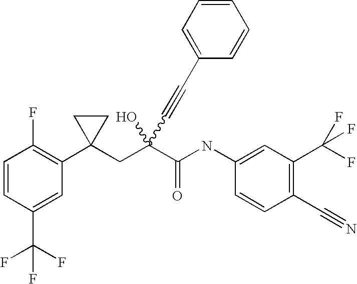

The compound described in Example 3b) was prepared from 3a) in analogy to Example 1b).

¹H-NMR (ppm, CDCl₃, 400 MHz): 0.87 (1H), 0.95-1.1 (3H), 2.40 (1H), 2.72 (1H), 3.02 (1H), 7.00 (1H), 7.25-7.42 (6H), 7.59 (1H), 7.72-7.83 (2H), 7.91 (1H), 8.87 (1H).

### EXAMPLE 3c AND 3d

### (+)-{2-Hydroxy-3-[1-(2-fluoro-5-trifluoromethylphenyl)cyclopropyl]-2-phenylethynyl)]propionic acid}(4-cyano-3-trifluoromethylphenyl)amide 3a and

### (−)-{2-hydroxy-3-[1-(2-fluoro-5-trifluoromethyl phenyl)cyclopropyl]-2-phenylethynyl)]propionic acid}(4-cyano-3-trifluoromethyl phenyl)amid 3b

The racemic mixture obtained in Example 3b was separated into the enantiomers 3c and 3d by preparative chiral HPLC (Chiralpak AD 250×10 mm column).

3c: [α]^{D}₂₀=+5.3° (CHCl₃, 9.6 mg/l ml; λ=589 nM)

3d: [α]^{D}₂₀=−5.7° (CHCl₃, 9.4 mg/l ml; λ=589 nM)

### EXAMPLE 4

### rac-{2-Hydroxy-3-[1-(2-fluoro-5-trifluoromethylphenyl)cyclopropyl]-2-(4-methyl phenyl)ethynyl)]propionic acid}(4-cyano-3-trifluoromethylphenyl)amide

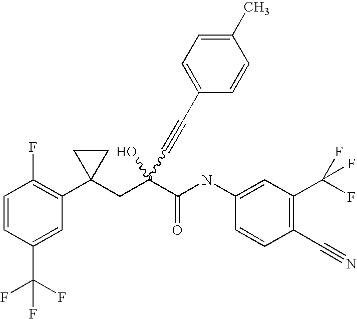

The compound described in Example 4 was prepared from 3a) in analogy to Example 2.

¹H-NMR (ppm, CDCl₃, 300 MHz): 0.83 (1H), 0.92-1.13 (3H), 2.33 (3H), 2.39 (1H), 2.73 (1H); 3.00 (1H), 7.00 (1H), 7.09 (2H), 7.20 (2H), 7.30 (1H), 7.57 (1H), 7.72-7.85 (2H), 7.90 (1H), 8.85 (1H).

### EXAMPLE 4a AND 4b

### (+)-{2-Hydroxy-3-[1-(2-fluoro-5-trifluoromethylphenyl)-cyclopropyl]-2-(4-methylphenylethynyl)]propionic acid}(4-cyano-3-trifluoromethylphenyl)amide 4a and

### (−)-{2-hydroxy-3-[1-(2-fluoro-5-trifluoromethyl phenyl)-cyclopropyl]-2-(4-methylphenylethynyl)]propionic acid}(4-cyano-3-trifluoromethylphenyl)amide 4b

The racemic mixture obtained in Example 4 was separated into the enantiomers 4a and 4b by preparative chiral HPLC (Chiralpak AD 250×10 mm column).

4a: [α]^{D}₂₀=+2.8° (CHCl₃, 10.0 mg/1 ml; λ=589 nM)

4b: [α]^{D}₂₀=−3.7° (CHCl₃, 10.5 mg/l ml; λ=589 nM)

### EXAMPLE 5

### rac-6-[4,4-Dimethyl-2-hydroxy-2-phenyl pentanoylamino]-4-methyl-2,3-benzoxazin-1-one

### 5a) {3-[1-(2-Fluoro-5-trifluoromethylphenyl)cyclopropyl]-2-oxopropionic acid}(4-nitro-3-trifluoromethylphenyl)amide

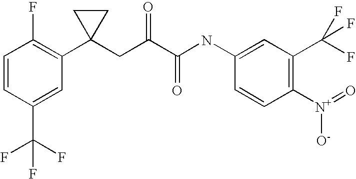

The compound described in Example 5a) was prepared from 3-[1-(2-fluoro-5-trifluoromethylphenyl)cyclopropyl]-2-oxopropionic acid and 4-nitro-3-trifluoromethyl-phenylamine in analogy to the process described in Example 1a).

¹H-NMR (ppm, CDCl₃, 400 MHz): 1.02 (4H), 3.31 (2H), 7.09 (1H), 7.04 (1H), 7.48 (1H), 7.70 (1H), 7.99 (2H), 8.05 1H), 8.97 (1H).

### 5b) rac-{2-Hydroxy-3-[1-(2-fluoro-5-trifluoromethylphenyl)-cyclopropyl]-2-phenylethynyl)]propionic acid}(4-nitro-3-trifluoromethylphenyl)amide

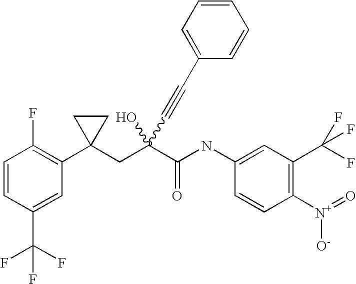

The compound described in Example 5b) was prepared from 5a) in analogy to Example 1b).

¹H-NMR (ppm, CDCl₃, 400 MHz): 0.87 (1H), 0.95-1.12 (3H), 2.40 (1H), 2.73 (1H), 3.01 (1H), 7.00 (1H), 7.23-7.40 (6H), 7.60 (1H), 7.82-7.99 (3H), 8.90 (1H).

### EXAMPLE 5c AND 5d

### (+)-{2-Hydroxy-3-[1-(2-fluoro-5-trifluoromethylphenyl)-cyclopropyl]-2-(4-methylphenylethynyl)]propionic acid}(4-nitro-3-trifluoromethylphenyl)amide 5a and

### (−)-{2-hydroxy-3-[1-(2-fluoro-5-trifluoromethylphenyl)-cyclopropyl]-2-(4-methylphenylethynyl)]propionic acid}(4-nitro-3-trifluoromethylphenyl)amide 5b

The racemic mixture obtained in Example 5b was separated into the enantiomers 5c and 5d by preparative chiral HPLC (Chiralpak AD 250×10 mm column).

5c: [α]^{D}₂₀=+5.9° (CHCl₃, 8.7 mg/l ml; λ=589 nM)

5d: [α]^{D}₂₀=−6.9° (CHCl₃, 9.0 mg/l ml; λ=589 nM)

### EXAMPLE 6

### rac-{2-Hydroxy-3-[1-(2-fluoro-5-trifluoromethylphenyl)-cyclopropyl]-2-(4-methyl phenyl)ethynyl)]propionic acid}(4-nitro-3-trifluoromethylphenyl)amide

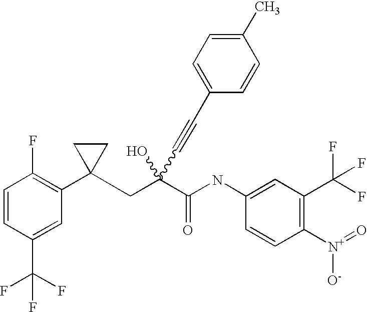

The compound described in Example 6 was prepared from 5a) in analogy to Example 2.

¹H-NMR (ppm, CDCl₃, 400 MHz): 0.85 (1H), 0.95-1.12 (3H), 2.32 (3H), 2.39 (1H), 2.72 (1H), 2.97 (1H), 7.01 (1H), 7.10 (2H), 7.21 (2H), 7.32 (1H), 7.60 (1H), 7.84-8.00 (3H), 8.90 (1H).

### EXAMPLE 7

### rac-{2-Hydroxy-3-[1-(2-fluoro-5-trifluoromethylphenyl)-cyclopropyl]-2-(4-methylphenyl)ethynyl)]propionic acid}(1-oxo-1H-1λ⁴-benzo[b]thiophen-5-yl)amide

### 7a) 5-Nitro-benzo[b]thiophene 1-oxide

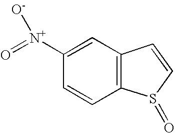

2.15 ml of hydrogen peroxide solution (30% strength in water) were added to 4.2 ml of trifluoroacetic acid at 23° C. After stirring at 23° C. for 30 minutes, a solution of 2 g of 5-nitrobenzo[b]thiophene in 15 ml of trifluoroacetic acid was slowly added. After stirring at 23° C. for one hour, the reaction mixture was poured into ice-water. It was then stirred for 3 hours. The precipitate was then filtered off with suction and washed with water. The resulting crude product was chromatographed on silica gel. 1.08 mg of product were obtained.

¹H-NMR (ppm, CDCl₃, 300 MHz): 7.32 (2H), 8.11 (1H), 8.36 (2H).

### 7b) 1-Oxo-1H-1λ⁴-benzo[b]thiophen-5-ylamine

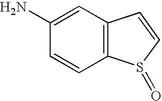

1.45 g of the compound obtained in 7a were suspended in 50 ml of ethanol. 8.38 g of tin(II) chloride dihydrate were added, and the mixture was stirred at 70° C. for 10 minutes. The reaction mixture was then poured into ice-cold saturated ammonium chloride solution. Stirring for 2 hours was followed by dilution with ethyl acetate and removal of the precipitated salts by filtration through Celite. The phases were then separated and the aqueous phase was extracted with ethyl acetate. The combined organic phases were washed with saturated aqueous sodium chloride solution, dried over sodium sulphate and concentrated in vacuo. The resulting crude product was chromatographed on silica gel. 505 mg of product were obtained.

¹H-NMR (ppm, DMSO-D₆, 300 MHz): 5.06 (2H), 6.71 (1H), 6.97 (1H), 7.15 (1H), 7.50-7.63 (2H).

### 7c) {3-[1-(2-Fluoro-5-trifluoromethylphenyl)cyclopropyl]-2-oxopropionic acid}(1-oxo-1H-1λ⁴-benzo[b]thiophen-5-yl)amide

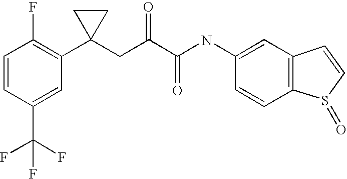

The compound described in Example 7c) was prepared from 3-[1-(2-fluoro-5-trifluoromethylphenyl)cyclopropyl]-2-oxopropionic acid and the compound described in 7b) in analogy to the process described in Example 1a).

¹H-NMR (ppm, CDCl₃, 300 MHz): 1.01 (4H), 3.35 (2H), 7.09 (1H), 7.30 (1H), 7.40 (1H), 7.48 (2H), 7.73 (1H), 7.82 (1H), 8.24 (1H), 8.74 (1H).

### 7d) rac-{2-Hydroxy-3-[1-(2-fluoro-5-trifluoromethylphenyl)-cyclopropyl]-2-(4-methylphenyl)ethynyl)]propionic acid}(1-oxo-1H-1λ⁴-benzo[b]thiophen-5-yl)amide

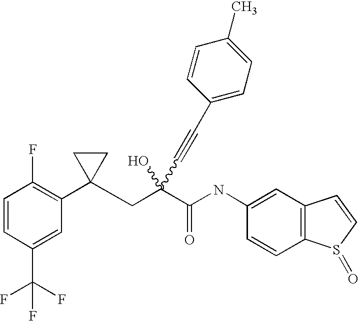

The compound described in Example 7d) was prepared from 7c) in analogy to Example 2.

¹H-NMR (ppm, CDCl₃, 300 MHz): 0.80-1.12 (4H), 2.33 (3H), 2.46 (1H), 2.59 (1H), 3.15 (1H), 6.96 (1H), 7.09 (2H), 7.21 (2H), 7.24-7.48 (3H), 7.48 (1H), 7.66 (1H), 7.80 (1H), 8.11 (1H), 8.50 (1H).

### EXAMPLE 8

### rac-{2-Hydroxy-3-[1-(2-fluoro-5-trifluoromethylphenyl)-cyclopropyl]-2-(4-methylphenyl)ethynyl)]propionic acid}(1,1-dioxo-2,3-dihydro-1H-1λ⁶-benzo[b]thiophen-5-yl)amide

### 8a) {3-[1-(2-Fluoro-5-trifluoromethylphenyl)-cyclopropyl]-2-oxopropionic acid}(1,1-dioxo-2,3-dihydro-1H-1λ⁶-benzo[b]thiophen-5-yl)amide

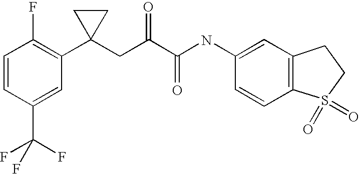

The compound described in Example 8a) was prepared from 3-[1-(2-fluoro-5-trifluoromethylphenyl)cyclopropyl]-2-oxopropionic acid and 1,1-dioxo-2,3-dihydro-1H-1λ⁶-benzo[b]thiophen-5-ylamine in analogy to the process described in Example 1a).

¹H-NMR (ppm, CDCl₃, 400 MHz): 1.02 (4H), 3.30 (2H), 3.37 (2H), 3.50 (2H), 7.09 (1H), 7.48 (2H), 7.71 (2H), 7.87 (1H), 8.83 (1H).

### 8b) rac-{2-Hydroxy-3-[1-(2-fluoro-5-trifluoromethylphenyl)-cyclopropyl]-2-(4-methylphenyl)ethynyl)]propionic acid}(1,1-dioxo-2,3-dihydro-1H-1λ⁶-benzo[b]thiophen-5-yl)amide

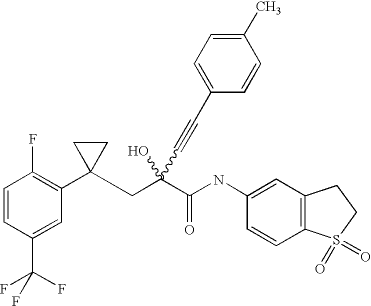

The compound described in Example 8b) was prepared from 8a) in analogy to Example 2.

¹H-NMR (ppm, CDCl₃, 300 MHz): 0.87 (1H), 0.92-1.12 (3H), 2.32 (3H), 2.43 (1H), 2.60 (1H), 3.04 (1H), 3.34 (2H), 3.50 (2H), 6.98 (1H), 7.09 (2H), 7.20 (2H), 7.34 (2H), 7.60 (1H), 7.67 (1H), 7.80 (1H), 8.70 (1H).

### EXAMPLE 9a AND 9b

### (+)-{2-Hydroxy-3-[1-(2-fluoro-5-trifluoromethylphenyl)-cyclopropyl]-2-(4-methylphenyl)-ethynyl)]propionic acid}(1,1-dioxo-2,3-dihydro-1H-1λ⁶-benzo[b]-thiophen-5-yl)amide 9a and

### (−)-{2-hydroxy-3-[1-(2-fluoro-5-trifluoromethylphenyl)cyclopropyl]-2-(4-methy)ethynyl)]propionic acid}(1,1-dioxo-2,3-dihydro-1H-1λ⁶-benzo[b]thiophen-5-yl)amide 9b

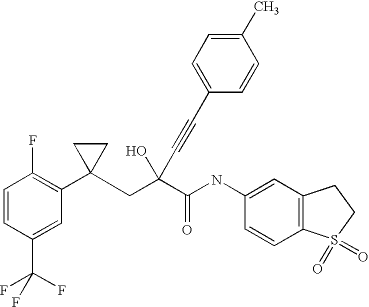

The racemic mixture obtained in Example 8 was separated into the enantiomers 9a and 9b by preparative chiral HPLC (Chiralpak AD 250×10 mm column).

9a: [α]^{D}₂₀: +20.6° (CHCl₃, 10.0 mg/1 ml; λ=589 nM)

9b: [α]^{D}₂₀: −20.7° (CHCl₃, 9.6 mg/1 ml; λ=589 nM)

### EXAMPLE 10

### rac-{2-Hydroxy-3-[1-(2-fluoro-5-trifluoromethylphenyl)cyclopropyl]-2-(4-methylphenyl)ethynyl)]propionic acid}(1,1-dioxo-1H-1λ⁶-benzo[b]thiophen-5-yl)amide

### 10a) {3-[1-(2-Fluoro-5-trifluoromethylphenyl)cyclopropyl]-2-oxopropionic acid}(1,1-dioxo-1H-1λ⁶-benzo[b]thiophen-5-yl)amide

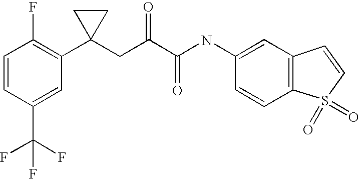

The compound described in Example 10a) was prepared from 3-[1-(2-fluoro-5-trifluoromethylphenyl)-cyclopropyl]-2-oxopropionic acid and 1,1-dioxo-1H-1λ⁶-benzo[b]thiophen-5-ylamine in analogy to the process described in Example 1a).

¹H-NMR (ppm, DMSO-D₆, 300 MHz): 0.92 (4H), 3.24 (2H), 7.28-7.38 (2H), 7.48 (2H), 7.74 (2H), 7.86 (1H), 8.01 (1H), 10.78 (1H).

### 10b) rac-{2-Hydroxy-3-[1-(2-fluoro-5-trifluoromethylphenyl)cyclopropyl]-2-(4-methylphenyl)-ethynyl)]propionic acid}(1,1-dioxo-1H-1λ⁶-benzo[b]thiophen-5-yl)amide

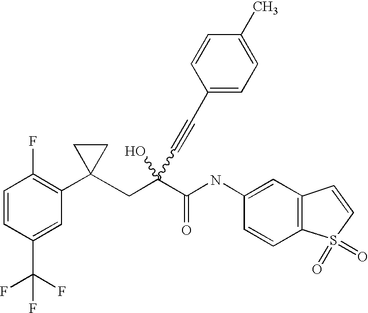

The compound described in Example 10) was prepared from 10a) in analogy to Example 2.

¹H-NMR (ppm, CDCl₃, 400 MHz): 0.86 (1H), 0.95-1.10 (3H), 2.32 (3H), 2.43 (1H), 2.62 (1H), 3.05 (1H), 6.72 (1H), 7.00 (1H), 7.07-7.25 (5H), 7.30 (1H), 7.48 (1H), 7.56-7.68 (2H), 7.80 (1H), 8.73 (1H).

### EXAMPLE 11

### rac-{2-Hydroxy-3-[1-(2-chloro-6-fluorophenyl)-dimethyl]-2-phenylethynyl)]-propionic acid}(4-cyano-3-trifluoromethyl)amide

### 11a) {3-[1-(2-Chloro-6-fluorophenyl)-dimethyl]-2-oxopropionic acid}(4-cyano-3-trifluoromethyl)amide

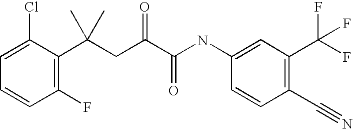

The compound described in Example 11a) was prepared from 3-[1-(2-chloro-6-fluorophenyl)dimethyl]-2-oxopropionic acid and 4-amino-2-trifluoromethylbenzonitrile in analogy to the process described in Example 1a).

¹H-NMR (ppm, CDCl₃, 300 MHz): 1.69 (3H), 1.71 (3H), 3.82 (2H), 6.94 (1H), 7.09-7.16

### 11b) rac-{2-Hydroxy-3-[1-(2-chloro-6-fluorophenyl)dimethyl]-2-phenylethynyl)]propionic acid}(4-cyano-3-trifluoromethyl)amide

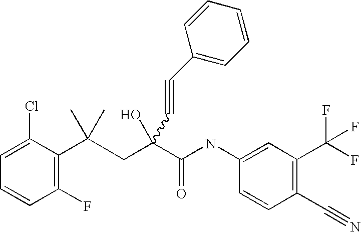

The compound described in Example 11b) was prepared from 11a) in analogy to Example 1b).

¹H-NMR (ppm, CDCl₃, 400 MHz): 1.77 (3H), 1.86 (3H), 2.93-3.04 (3H), 6.86 (1H), 6.97 (1H), 7.06 (1H), 7.31-7.36 (5H), 7.79-7.88 (2H), 8.02 (1H), 8.89 (1H).

### EXAMPLE 12

### rac-{2-Hydroxy-3-[1-(2-chloro-6-fluorophenyl)dimethyl]-2-phenylethynyl)]-propionic acid}(4-cyano-3-chlorophenyl)amide

### 12a) {3-[1-(2-Chloro-6-fluorophenyl)dimethyl]-2-oxopropionic acid}(4-cyano-3-chlorophenyl)amide

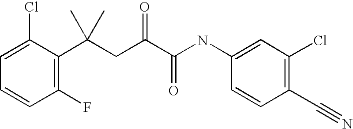

The compound described in Example 12a) was prepared from 3-[1-(2-chloro-6-fluorophenyl)dimethyl]-2-oxopropionic acid and 4-amino-2-chlorobenzonitrile in analogy to the process described in Example 1a).

¹H-NMR (ppm, CDCl₃, 300 MHz): 1.69 (3H), 1.71 (3H), 3.80 (2H), 6.94 (1H), 7.07-7.17 (2H), 7.52 (1H), 7.64 (1H), 7.95 (1H), 8.85 (1H).

### 12b) rac-{2-Hydroxy-3-[1-(2-chloro-6-fluorophenyl)dimethyl]-2-phenylethynyl)]propionic acid}(4-cyano-3-chlorophenyl)amide

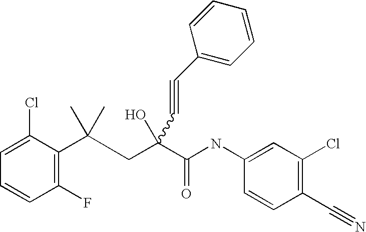

The compound described in Example 12b) was prepared from 12a) in analogy to Example 1b).

¹H-NMR (ppm, CDCl₃, 400 MHz): 1.72 (3H), 1.80 (3H), 2.92 (2H), 3.04 (1H), 6.81 (1H), 6.94 (1H), 7.03 (1H), 7.26-7.43 (6H), 7.58 (1H), 7.82 (1H), 8.72 (1H).

### EXAMPLE 12c AND 12d

### (+)-{2-Hydroxy-3-[1-(2-chloro-6-fluorophenyl)dimethyl]-2-phenylethynyl]propionic acid}(4-cyano-3-chlorophenyl)amide 12a and

### (−)-{2-hydroxy-3-[1-(2-chloro-6-fluorophenyl)dimethyl]-2-phenylethynyl]propionic acid}(4-cyano-3-chlorophenyl)amide 12b

The racemic mixture obtained in Example 12b was separated into the enantiomers 12c and 12d by preparative chiral HPLC (Chiralpak AD 250×10 mm column).

12c: [α]^{D}₂₀=+13.9° (CHCl₃, 10.6 mg/l ml; λ=589 nM)

12d: [α]^{D}₂₀=−14.0° (CHCl₃, 10.8 mg/l ml; λ=589 nM)

### EXAMPLE 13

### rac-{2-Hydroxy-3-[1-(2-chloro-6-fluorophenyl)dimethyl]-2-phenylethynyl)]-propionic acid}(4-nitro-3-trifluoromethylphenyl)amide

### 13a) {3-[1-(2-Chloro-6-fluorophenyl)dimethyl]-2-oxopropionic acid}(4-cyano-3-trifluoromethylphenyl)amide

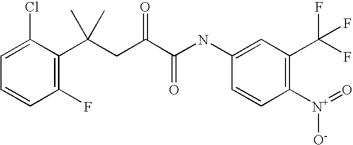

The compound described in Example 12a) was prepared from 3-[1-(2-chloro-6-fluorophenyl)dimethyl]-2-oxopropionic acid and 4-nitro-3-trifluoromethylaniline in analogy to the process described in Example 1a).

¹H-NMR (ppm, CDCl₃, 300 MHz): 1.70 (3H), 1.71 (3H), 3.82 (2H), 6.92 (1H), 7.08-7.17 (2H), 8.00 (2H), 8.09 (1H), 9.01 (1H).

### 13b) rac-{2-Hydroxy-3-[1-(2-chloro-6-fluorophenyl)dimethyl]-2-phenylethynyl)]propionic acid}(4-nitro-3-trifluoromethylphenyl)amide

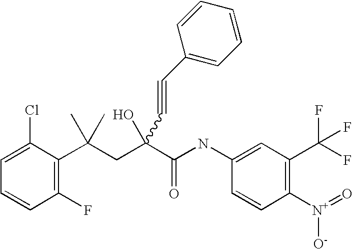

The compound described in Example 13b) was prepared from 13a) in analogy to Example 1b).

¹H-NMR (ppm, CDCl₃, 400 MHz): 1.72 (3H), 1.81 (3H), 2.95 (2H), 3.01 (1H), 6.78-7.03 (3H), 7.27-7.39 (5H), 7.86-7.96 (3H), 8.90 (1H).

### EXAMPLE 14

### rac-{2-Hydroxy-3-[1-(2-fluoro-5-trifluoromethylphenyl)cyclopropyl]-2-phenylethynyl)]propionic acid}(4-cyanophenyl)amide

### 14a) {3-[1-(2-Fluoro-5-trifluoromethylphenyl)cyclopropyl]-2-oxopropionic acid}(4-cyanophenyl)amide

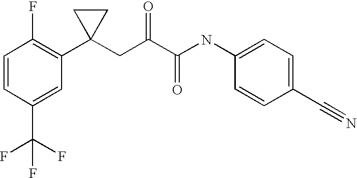

The compound described in Example 14a) was prepared from 3-[1-(2-fluoro-5-trifluoromethylphenyl)cyclopropyl]-2-oxopropionic acid and 4-aminobenzonitrile in analogy to the process described in Example 1a).

¹H-NMR (ppm, CDCl₃, 300 MHz): 1.01 (4H), 3.31 (2H), 7.09 (1H), 7.48 (1H), 7.63-7.73 (5H), 8.79 (1H).

### 14b) rac-{2-Hydroxy-3-[1-(2-fluoro-5-trifluoromethylphenyl)cyclopropyl]-2-phenylethynyl)]propionic acid}(4-cyanophenyl)amide

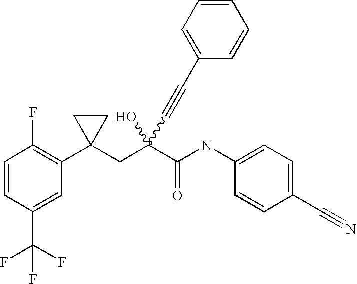

The compound described in Example 14b) was prepared from 14a) in analogy to Example 1b).

¹H-NMR (ppm, CDCl₃, 400 MHz): 0.86-0.90 (1H), 0.97-1.08 (3H), 2.45 (1H), 2.64 (1H), 3.05 (1H), 7.00 (1H), 7.29-7.35 (6H), 7.60-7.63 (5H), 8.68 (1H).

### EXAMPLE 15

### rac-2-{Hydroxy-3-[1-(2-fluoro-5-trifluoromethylphenyl)-cyclopropyl]-2-phenylethynyl)]propionic acid}phenylamide

### 15a) {3-[1-(2-fluoro-5-trifluoromethylphenyl)cyclopropyl]-2-oxopropionic acid}phenylamide

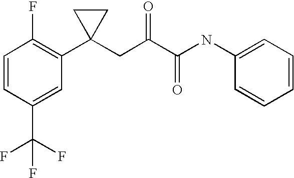

The compound described in Example 15a) was prepared from 3-[1-(2-fluoro-5-trifluoromethylphenyl)cyclopropyl]-2-oxopropionic acid and aniline in analogy to the process described in Example 1a).

¹H-NMR (ppm, CDCl₃, 300 MHz): 1.00 (4H), 3.33 (2H), 7.09 (1H), 7.16 (1H), 7.35 (2H), 7.48 (1H), 7.58 (2H), 7.73 (1H), 8.61 (1H).

### 15b) rac-{2-Hydroxy-3-[1-(2-fluoro-5-trifluoromethylphenyl)-cyclopropyl]-2-phenylethynyl)]-propionic acid}phenylamide

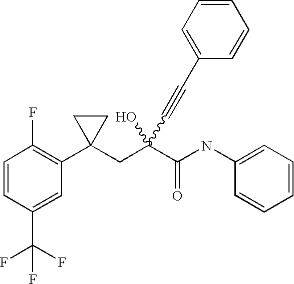

The compound described in Example 15b) was prepared from 15a) in analogy to Example 1b).

¹H-NMR (ppm, CDCl₃, 400 MHz): 0.85-1.09 (4H), 2.47 (1H), 2.57 (1H), 3.17 (1H), 6.98 (1H), 7.14 (1H), 7.28-7.35 (8H), 7.50 (2H), 7.64 (1H), 8.40 (1H).

### EXAMPLE 16

### rac-{2-Hydroxy-3-[1-(2-fluoro-5-trifluoromethylphenyl)cyclopropyl]-2-(3-hydroxy-propynyl)]propionic acid}(4-cyano-3-trifluoromethylphenyl)amide

### 16a) {3-[1-(2-Fluoro-5-trifluoromethylphenyl)cyclopropyl]-2-(3-tert-butydimethylsilyloxy-propynyl)propionic acid}(4-cyano-3-trifluoromethylphenyl)amide

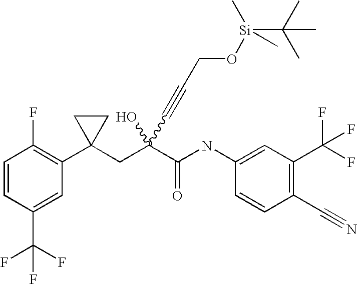

The compound described in Example 16a) was prepared from {3-[1-(2-fluoro-5-trifluoromethylphenyl)cyclopropyl]-2-oxopropionic acid}(4-cyano-3-trifluoromethylphenyl)amide (see Example 3a) and 3-tert-butylsilyloxypropyne in analogy to the process described in Example 1b).

¹H-NMR (ppm, CDCl₃, 300 MHz): 0.07 (6H), 0.76-0.84 (1H), 0.88 (9H), 1.07-0.92 (3H), 2.24 (1H), 2.69 (1H), 3.11 (1H), 4.23 (2H), 7.02 (1H), 7.31-7.36 (1H), 7.54 (1H), 7.76 (2H), 7.85 (1H), 8.82 (1H).

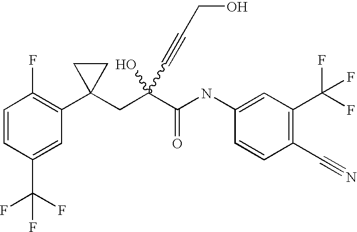

### 16b) rac-{2-Hydroxy-3-[1-(2-fluoro-5-trifluoromethylphenyl)cyclopropyl]-2-(3-hydroxypropynyl)]propionic acid}(4-cyano-3-trifluoromethylphenyl)amide

Tetrabutylammonium fluoride (280 μL, 1M in THF) was added to a solution of the compound (170 mg) described in 16a) in 5 ml of THF. The mixture was stirred at 23° C. for 4 h. The reaction mixture was then poured into saturated sodium bicarbonate solution and extracted with ethyl acetate. The combined organic phases were washed with saturated sodium chloride solution, dried over sodium sulphate and concentrated.

The crude product was chromatographed on silica gel. 137 mg of product are obtained.

¹H-NMR (ppm, CDCl₃, 400 MHz): 0.81-0.86 (1H), 0.90-1.02 (3H), 1.25 (1H), 2.30 (1H), 2.64 (1H), 4.17 (2H), 7.04 (1H), 7.36 (1H), 7.54 (1H), 7.77 (2H), 7.89 (1H), 8.87 (1H).

### EXAMPLE 16c AND 16d

### (+)-{2-Hydroxy-3-[1-(2-fluoro-5-trifluoromethylphenyl)cyclopropyl]-2-(3-hydroxypropynyl)]propionic acid}(4-cyano-3-trifluoromethylphenyl)amide 16c and

### (−)-{2-hydroxy-3-[1-(2-fluoro-5-trifluoromethyl phenyl)cyclopropyl]-2-(3-hydroxypropynyl)]propionic acid}(4-cyano-3-trifluoromethyl phenyl)amide 16d

The racemic mixture obtained in Example 16b was separated into the enantiomers 16c and 16d by preparative chiral HPLC (Chiralpak AD 250×10 mm column).

16c: [α]^{D}₂₀=+36.9° (CHCl₃, 10.1 mg/l ml; λ=589 nM)

16d: [α]^{D}₂₀=−37.9° (CHCl₃, 10.2 mg/l ml; λ=589 nM)

### EXAMPLE 17

### rac-{2-Hydroxy-3-[1-(2-fluoro-5-trifluoromethylphenyl)cyclopropyl]-2-(1-pentynyl)]propionic acid}(4-cyano-3-trifluoromethyl phenyl)amide

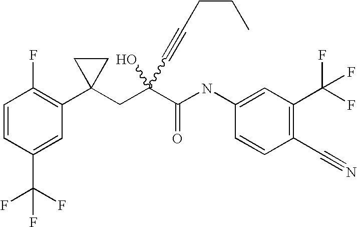

The compound described in Example 17 was prepared from {3-[1-(2-fluoro-5-trifluoromethylphenyl)cyclopropyl]-2-oxopropionic acid}(4-cyano-3-trifluoromethylphenyl)amide and 1-pentyne in analogy to the process described in Example 1b).

¹H-NMR (ppm, CDCl₃, 400 MHz): 0.83-0.90 (1H), 0.96-1.07 (6H), 1.52 (2H), 2.15 (2H), 2.29 (1H), 2.68 (1H), 2.83 (1H), 7.09 (1H), 7.41 (1H), 7.59 (1H), 7.81 (2H), 7.93 (1H), 8.85 (1H).

### EXAMPLE 17a AND 17b

### (+)-{2-Hydroxy-3-[1-(2-fluoro-5-trifluoromethylphenyl)cyclopropyl]-2-(1-pentynyl)]-propionic acid}(4-cyano-3-trifluoromethylphenyl)amide 17a and

### (−)-{2-hydroxy-3-[1-(2-fluoro-5-trifluoromethyl phenyl)cyclopropyl]-2-(1-pentynyl)]-propionic acid}(4-cyano-3-trifluoromethylphenyl)amide 17b

The racemic mixture obtained in Example 17 was separated into the enantiomers 3a and 3b by preparative chiral HPLC (Chiralpak AD 250×10 mm column).

3a: [α]^{D}₂₀=+27.4° (CHCl₃, 21.5 mg/1 ml; λ=589 nM)

3b: [α]^{D}₂₀=−27.1° (CHCl₃, 21.9 mg/l ml; λ=589 nM)

### EXAMPLE 18

### rac-2-{Hydroxy-3-[1-(2-fluoro-5-trifluoromethylphenyl)cyclopropyl]-2-phenylethynyl)]propionic acid}(3-trifluoromethylphenyl)amide

### 18a) {3-[1-(2-Fluoro-5-trifluoromethylphenyl)cyclopropyl]-2-oxopropionic acid}(3-trifluoromethylphenyl)amide

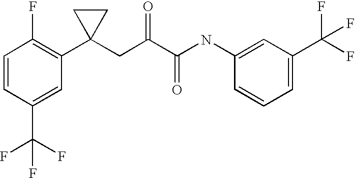

The compound described in Example 18a) was prepared from 3-[1-(2-fluoro-5-trifluoromethylphenyl)cyclopropyl]-2-oxopropionic acid and 3-trifluoromethylaniline in analogy to the process described in Example 1a).

¹H-NMR (ppm, CDCl₃, 300 MHz): 1.01 (4H), 3.32 (2H), 7.10 (1H), 7.33 (1H), 7.41-7.53 (3H), 7.73 (1H), 7.92 (1H), 8.73 (1H).

### 18b) rac-{2-Hydroxy-3-[1-(2-fluoro-5-trifluoromethylphenyl)-cyclopropyl]-2-phenylethynyl)]-propionic acid}(3-trifluoromethylphenyl)amide

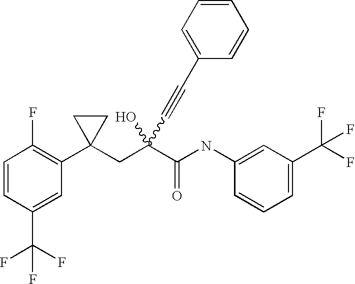

The compound described in Example 18b) was prepared from Example 18a) in analogy to Example 1b).

¹H-NMR (ppm, CDCl₃, 400 MHz): 0.87-0.94 (1H), 1.01-1.13 (3H), 2.49 (1H), 2.70 (1H), 3.14 (1H), 7.04 (1H), 7.32-7.51 (8H), 7.68 (2H), 7.82 (1H), 8.61 (1H).

### EXAMPLE 19

### rac-{2-Hydroxy-3-[1-(2-fluoro-5-trifluoromethylphenyl)cyclopropyl]-2-(4-hydroxyphenylethynyl)]propionic acid}(4-cyano-3-trifluoromethylphenyl)amide

### 19a) rac-{2-Hydroxy-3-[1-(2-fluoro-5-trifluoromethylphenyl)cyclopropyl]-2-trimethylsilylethynyl]propionic acid}(4-cyano-3-trifluoromethylphenyl)amide

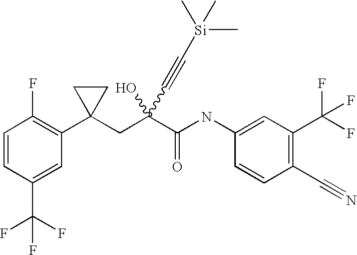

The compound described in Example 19a) was prepared from {3-[1-(2-fluoro-5-trifluoromethylphenyl)-cyclopropyl]-2-oxopropionic acid}(4-cyano-3-trifluoromethylphenyl)amide and trimethylsilylacetylene in analogy to Example 1b).

¹H-NMR (ppm, CDCl₃, 400 MHz): 0.22 (9H), 0.76-0.86 (1H), 0.98-1.14 (3H), 2.28 (1H), 2.74 (1H), 2.87 (1H), 7.08 (1H), 7.42 (1H), 7.61 (1H), 7.80 (2H), 7.92 (1H), 8.85 (1H).

### 19b) rac-{2-Hydroxy-3-[1-(2-fluoro-5-trifluoromethylphenyl)cyclopropyl]-2-ethynyl]-propionic acid}(4-cyano-3-trifluoromethylphenyl)amide

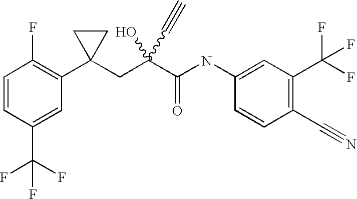

The compound described in Example 19b) was prepared from 19a) in analogy to Example 16b).

¹H-NMR (ppm, CDCl₃, 400 MHz): 0.81-0.88 (1H), 0.92-1.06 (3H), 2.30 (1H), 2.58 (1H), 2.69 (1H), 3.15 (1H), 7.03 (1H), 7.36 (1H), 7.54 (1H), 7.78 (2H), 7.88 (1H), 8.78 (1H).

### 19c) rac-{2-Hydroxy-3-[1-(2-fluoro-5-trifluoromethylphenyl)-cyclopropyl]-2-(4-acetoxyphenylethynyl)]-propionic acid}(4-cyano-3-trifluoromethylphenyl)amide

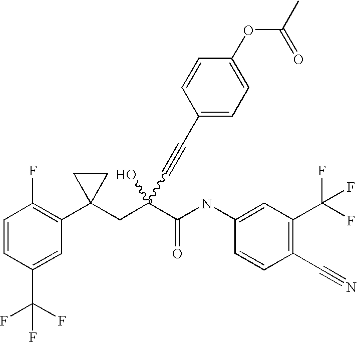

Palladium(II) acetate (3.7 mg), triphenylphosphine (8.7 mg) and copper(I) iodide (6.9 mg) were added to a solution of triethylamine (3.9 ml) in THF (7 ml). The mixture was stirred for 2 minutes. Then 4-acetoxyiodobenzene (64 mg) was added. The mixture was stirred for 5 minutes. Then the compound (80 mg) described in 19b) was added, and reaction was allowed to take place in an ultrasonic bath for 2 hours. The reaction mixture was then poured into ice-cold saturated ammonium chloride solution. It was extracted with ethyl acetate. The combined organic phases were washed with saturated sodium chloride solution, dried over sodium sulphate and concentrated. The crude product was chromatographed on silica gel and then chromatographed with HPLC. 23 mg of product were obtained.

¹H-NMR (ppm, CDCl₃, 400 MHz): 0.88-0.94 (1H), 1.02-1.13 (3H), 2.34 (3H), 2.44 (1H), 2.77 (1H), 3.10 (1H), 7.03-7.10 (3H), 7.34-7.40 (3H), 7.63 (1H), 7.84 (2H), 7.96 (1H), 8.90 (1H).

### 19d) rac-{2-Hydroxy-3-[1-(2-fluoro-5-trifluoromethylphenyl)cyclopropyl]-2-(4-hydroxyphenylethynyl)]propionic acid}(4-cyano-3-trifluoromethylphenyl)amide

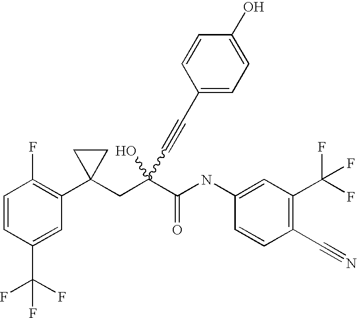

A solution of the compound (18 mg) described in 19c) and sodium bicarbonate (41 mg) in MeOH (1 ml) was stirred for 2 hours. The reaction mixture was diluted with ethyl acetate. The combined organic phases were washed with saturated sodium chloride solution, dried over sodium sulphate and concentrated. The crude product was chromatographed by preparative TLC. 11 mg of product were obtained.

¹H-NMR (ppm, CDCl₃, 400 MHz): 0.83-0.88 (1H), 0.96-1.09 (3H), 2.38 (1H), 2.71 (1H), 2.98 (1H), 5.17 (1H), 6.75 (2H), 7.01 (1H), 7.21 (2H), 7.32 (1H), 7.58 (1H), 7.79 (2H), 7.91 (1H), 8.87 (1H).

### EXAMPLE 20

### rac-{2-Hydroxy-3-[1-(2-chlorophenyl)cyclopropyl]-2-phenylethynyl)]propionic acid}(4-cyano-3-trifluoromethylphenyl)amide

### 20a) {3-[1-(2-Chlorophenyl)cyclopropyl]-2-oxopropionic acid}(4-cyano-3-trifluoromethylphenyl)amide

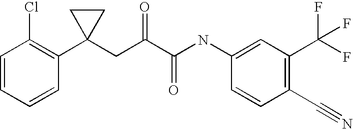

The compound described in Example 20a) was prepared from 3-[1-(2-chlorophenyl)cyclopropyl]-2-oxopropionic acid and 4-amino-2-trifluoromethylbenzonitrile in analogy to the process described in Example 1a).

¹H-NMR (ppm, CDCl₃, 300 MHz): 1.02 (4H), 3.36 (2H), 7.15-7.19 (2H), 7.32 (1H), 7.47 (1H), 7.82 (1H), 7.92 (1H), 8.04 (1H), 8.94 (1H).

### 20b) rac-{2-Hydroxy-3-[1-(2-chlorophenyl)cyclopropyl]-2-phenylethynyl]propionic acid}(4-cyano-3-trifluoromethylphenyl)amide

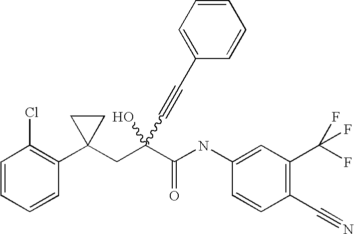

The compound described in Example 20b) was prepared from 20a) in analogy to Example 1b).

¹H-NMR (ppm, CDCl₃, 400 MHz): 0.80-0.88 (1H), 0.96-1.03 (1H), 1.09-1.28 (2H), 2.94 (2H), 7.04-7.14 (2H), 7.27-7.48 (8H), 7.79 (2H), 7.93 (1H), 8.80 (1H).

### EXAMPLE 20c AND 20d

### (+)-{2-Hydroxy-3-[1-(2-chlorophenyl)cyclopropyl]-2-phenylethynyl)]-propionic acid}(4-cyano-3-trifluoromethylphenyl)amide 20a and

### (−)-{2-hydroxy-3-[1-(2-chlorophenyl)cyclopropyl]-2-phenylethynyl)]propionic acid}(4-cyano-3-trifluoromethylphenyl)amide 20b

The racemic mixture obtained in Example 20b was separated into the enantiomers 20c and 20d by preparative chiral HPLC (Chiralpak AD 250×10 mm column).

20c: [α]^{D}₂₀=+17.9° (CHCl₃, 10.4 mg/l ml; λ=589 nM)

20d: [α]^{D}₂₀=−17.5° (CHCl₃, 10.3 mg/l ml; λ=589 nM)

### EXAMPLE 21

### rac-{2-Hydroxy-3-[1-(2-chlorophenyl)cyclopropyl]-2-phenylethynyl)]propionic acid}(4-cyano-3-chlorophenyl)amide

### 21a) {3-[1-(2-Chlorophenyl)cyclopropyl]-2-oxopropionic acid}(4-cyano-3-chlorophenyl)amide

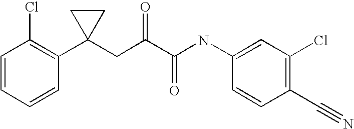

The compound described in Example 21a) was prepared from 3-[1-(2-chlorophenyl)cyclopropyl]-2-oxopropionic acid and 4-amino-2-chlorobenzonitrile in analogy to the process described in Example 1a).

¹H-NMR (ppm, CDCl₃, 300 MHz): 1.01 (4H), 3.35 (2H), 7.15-7.18 (2H), 7.32 (1H), 7.45-7.53 (2H), 7.64 (1H), 7.91 (1H), 8.81 (1H).

### 21b) rac-{2-Hydroxy-3-[1-(2-Chlorophenyl)cyclopropyl]-2-phenylethynyl)]propionic acid}-(4-cyano-3-chlorophenyl)amide

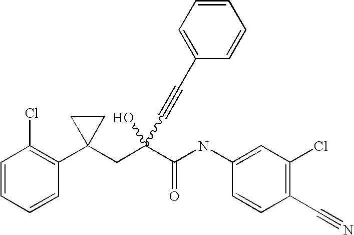

The compound described in Example 21b) was prepared from 21a) in analogy to Example 1b).

¹H-NMR (ppm, CDCl₃, 400 MHz): 0.83 (1H), 1.00 (1H), 1.08-1.20 (2H), 2.89 (1H), 7.07-7.15 (2H), 7.29-7.49 (8H), 7.59 (1H), 7.81 (1H), 8.86 (1H).

### EXAMPLE 21c AND 21d

### (+)-{2-Hydroxy-3-[1-(2-chlorophenyl)cyclopropyl]-2-phenylethynyl)]propionic acid}(4-cyano-3-chlorophenyl)amide 21c and

### (−)-{2-hydroxy-3-[1-(2-chlorophenyl)cyclopropyl]-2-phenylethynyl)]propionic acid}(4-cyano-3-chlorophenyl)amide 21d

The racemic mixture obtained in Example 21b was separated into the enantiomers 21c and 21d by preparative chiral HPLC (Chiralpak AD 250×10 mm column).

21c: [α]^{D}₂₀=+26.9° (CHCl₃, 10.3 mg/l ml; λ=589 nM)

21d: [α]^{D}₂₀=−26.5° (CHCl₃, 10.4 mg/l ml; λ=589 nM)

### EXAMPLE 22

### rac-{2-Hydroxy-3-[1-(2-chlorophenyl)cyclopropyl]-2-phenylethynyl)]propionic acid}(4-nitro-3-trifluoromethylphenyl)amide

### 22a) {3-[1-(2-Chlorophenyl)cyclopropyl]-2-oxopropionic acid}(4-nitro-3-trifluoromethylphenyl)amide

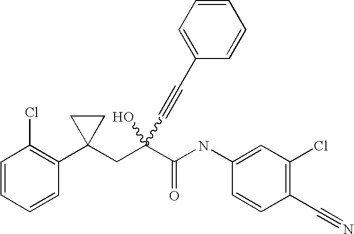

The compound described in Example 22a) was prepared from 3-[1-(2-chlorophenyl)cyclopropyl]-2-oxopropionic acid and 4-nitro-3-trifluoromethylaniline in analogy to the process described in Example 1a).

¹H-NMR (ppm, CDCl₃, 300 MHz): 1.07 (4H), 3.41 (2H), 7.20-7.24 (2H), 7.37 (1H), 7.52 (1H), 8.03 (2H), 8.09 (1H), 9.01 (1H).

### 22b) rac-{2-Hydroxy-3-[1-(2-chlorophenyl)cyclopropyl]-2-phenylethynyl)]propionic acid}(4-nitro-3-trifluoromethylphenyl)amide

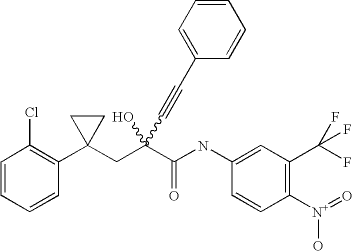

The compound described in Example 22b) was prepared from 22a) in analogy to Example 1b).

¹H-NMR (ppm, CDCl₃, 400 MHz): 0.85 (1H), 1.01 (1H), 1.12-1.20 (2H), 2.93 (2H), 7.06-7.14 (2H), 7.28-7.48 (7H), 7.87-7.97 (3H), 8.84 (1H).

### EXAMPLE 23

### rac-{2-Hydroxy-3-[1-(2-fluoro-5-trifluoromethylphenyl)cyclopropyl]-2-dimethylaminopropyne]propionic acid}(4-cyano-3-trifluoromethylphenyl)amide

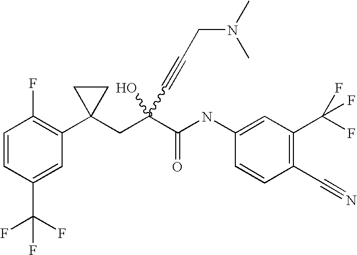

The compound described in Example 23 was prepared from {3-[1-(2-fluoro-5-trifluoromethylphenyl)cyclopropyl]-2-oxopropionic acid}(4-cyano-3-trifluoromethylphenyl)amide and 3-(N,N-dimethylamino)propyne in analogy to Example 1b).

¹H-NMR (ppm, CDCl₃, 400 MHz): 0.80-0.87 (1H), 0.93-1.07 (3H), 2.26-2.31 (7H), 2.74 (1H), 3.19 (2H), 7.06 (1H), 7.37 (1H), 7.56 (1H), 7.82 (2H), 7.94 (1H), 9.03 (1H).

### EXAMPLE 24

### rac-{2-Hydroxy-3-[1-(2-fluoro-5-trifluoromethylphenyl)cyclopropyl]-2-(1-methyl-1H-imidazol-5-ylethynyl)]propionic acid}(4-cyano-3-trifluoromethylphenyl)amide

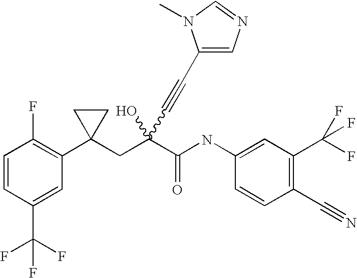

The compound described in Example 24 was prepared from {3-[1-(2-fluoro-5-trifluoromethylphenyl)cyclopropyl]-2-oxopropionic acid}(4-cyano-3-trifluoromethylphenyl)amid and 1-methyl-1-imidazol-5-ylethyne in analogy to Example 1b).

¹H-NMR (ppm, CDCl₃, 400 MHz): 0.77-0.84 (1H), 0.91-1.05 (3H), 2.28 (1H), 2.81 (1H), 3.57 (3H), 7.01 (1H), 7.09 (1H), 7.28 (1H), 7.38 (1H), 7.52 (1H), 7.73-7.81 (2H), 7.92 (1H), 9.24 (1H).

### EXAMPLE 24a AND 24b

### (+)-{2-Hydroxy-3-[1-(2-fluoro-5-trifluoromethylphenyl)cyclopropyl]-2-(1-methyl-1H-imidazol-5-ylethynyl)]propionic acid}(4-cyano-3-trifluoromethyl phenyl)amide 24a and

### (−){2-hydroxy-3-[1-(2-fluoro-5-trifluoromethylphenyl)cyclopropyl]-2-(1-methyl-1H-imidazol-5-ylethynyl)]-propionic acid}(4-cyano-3-trifluoromethylphenyl)amide 24b

The racemic mixture obtained in Example 24 was separated into the enantiomers 24a and 24b by preparative chiral HPLC (Chiralpak AD 250×10 mm column).

24a: [α]^{D}₂₀=+41.7° (CHCl₃, 10.3 mg/l ml; λ=589 nM)

24b: [α]^{D}₂₀=−42.9° (CHCl₃, 10.5 mg/l ml; λ=589 nM)

### EXAMPLE 25

### rac-{2-Hydroxy-3-[1-(2-fluoro-5-trifluoromethylphenyl)cyclopropyl]-2-(2-pyridylethynyl)]propionic acid}(4-cyano-3-trifluoromethylphenyl)amide

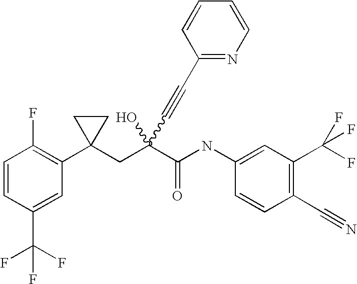

The compound described in Example 25 was prepared from {3-[1-(2-fluoro-5-trifluoromethylphenyl)cyclopropyl]-2-oxopropionic acid}(4-cyano-3-trifluoromethylphenyl)amide and 2-pyridinylethyne in analogy to Example 1b).

¹H-NMR (ppm, CDCl₃, 400 MHz): 0.78-0.83 (1H), 0.92-1.03 (3H), 2.45 (1H), 2.75 (1H), 5.39 (1H), 6.95 (1H), 7.24 (1H), 7.27-7.34 (2H), 7.54 (1H), 7.67 (1H), 7.74 (1H), 7.82 (1H), 7.94 (1H), 8.42 (1H), 9.34 (1H).

### EXAMPLE 26

### rac-{2-Hydroxy-3-[1-(2-fluoro-5-trifluoromethylphenyl)cyclopropyl]-2-(4-carboxyethynyl)]propionic acid}(4-cyano-3-trifluoromethylphenyl)amide

### 26a) rac-{2-Hydroxy-3-[1-(2-fluoro-5-trifluoromethylphenyl)cyclopropyl]-2-(4-methoxycarbonylethynyl)]propionic acid}(4-cyano-3-trifluoromethylphenyl)amide

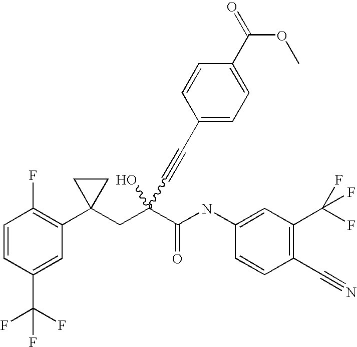

The compound described in Example 26a) was prepared from 19b) and methyl 4-iodobenzoate in analogy to Example 19c).

¹H-NMR (ppm, CDCl₃, 400 MHz): 0.85-0.92 (1H), 0.96-1.06 (3H), 2.44 (1H), 2.62 (1H), 3.18 (1H), 3.92 (3H), 7.01 (1H), 7.21-7.38 (3H), 7.58 (1H), 7.75-7.83 (2H), 7.92 (1H), 7.94 (2H), 8.84 (1H).

### 26b) rac-{2-Hydroxy-3-[1-(2-fluoro-5-trifluoromethylphenyl)cyclopropyl]-2-(4-carboxyethynyl)]propionic acid}(4-cyano-3-trifluoromethylphenyl)amide

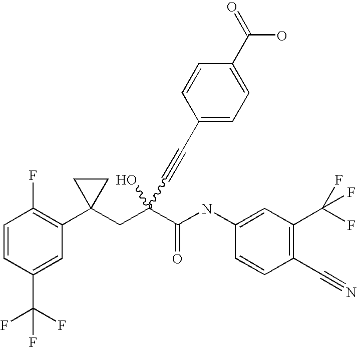

A solution of the compound (40 mg) described in 26a) and sodium hydroxide (2M aq, 90 μl) in THF (2 ml) and EtOH (1 ml) was stirred at 23° C. for 16 hours. The reaction mixture was mixed with HCl (2N aq, 350 μl) and extracted with dichloromethane. The combined organic phases were washed with saturated sodium chloride solution, dried over sodium sulphate and concentrated. The crude product was chromatographed by preparative TLC. 15 mg of product are obtained.

¹H-NMR (ppm, DMSO-d₆, 400 MHz): 0.60-0.66 (1H), 0.94-1.00 (2H), 1.10-1.16 (1H), 2.05 (1H), 2.94 (1H), 7.22 (1H), 7.33 (1H), 7.37 (2H), 7.53-7.67 (2H), 7.88 (2H), 8.04 (2H), 8.20 (1H), 10.67 (1H).

### EXAMPLE 26c AND 26d

### (+)-{2-Hydroxy-3-[1-(2-fluoro-5-trifluoromethylphenyl)cyclopropyl]-2-(4-carboxyethynyl)]propionic acid}(4-cyano-3-trifluoromethylphenyl)amide 26c and

### (−)-{2-hydroxy-3-[1-(2-fluoro-5-trifluoromethylphenyl)cyclopropyl]-2-(4-carboxyethynyl)]propionic acid}(4-cyano-3-trifluoromethylphenyl)amide 26d

The racemic mixture obtained in Example 26b was separated into the enantiomers 26c and 26d by preparative chiral HPLC (Chiralpak AD 250×10 mm column).

26c: [α]^{D}₂₀=+3.8° (CHCl₃, 5.2 mg/l ml; λ=589 nM)

26d: [α]^{D}₂₀=−2.4° (CHCl₃, 5.2 mg/l ml; λ=589 nM)

### EXAMPLE 27

### rac-2-{Hydroxy-3-[1-(2-fluoro-5-trifluoromethylphenyl)cyclopropyl]-2-phenyl)]-propionic acid}(3,4-dichlorophenyl)amide

### 27a) {3-[1-(2-Fluoro-5-trifluoromethylphenyl)cyclopropyl]-2-oxopropionic acid}(3,4-dichlorophenyl)amide

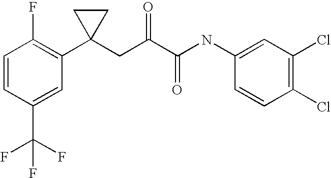

The compound described in Example 27a) was prepared from 3-[1-(2-fluoro-5-trifluoromethylphenyl)cyclopropyl]-2-oxopropionic acid and 3,4-dichloroaniline in analogy to the process described in Example 1a).

¹H-NMR (ppm, CDCl₃, 300 MHz): 1.00 (4H), 3.30 (2H), 7.09 (1H), 7.40 (2H), 7.48 (1H), 7.71 (1H), 7.84 (1H), 8.62 (1H).

### 27b) rac-{2-Hydroxy-3-[1-(2-fluoro-5-trifluoromethylphenyl)cyclopropyl]-2-phenylethynyl)]propionic acid}(3,4-dichlorophenyl)amide

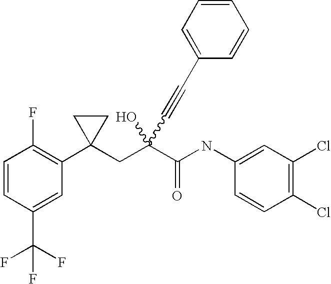

The compound described in Example 27b) was prepared from 27a) in analogy to Example 1b).

¹H-NMR (ppm, CDCl₃, 400 MHz): 0.90-0.94 (1H), 1.02-1.13 (3H), 2.49 (1H), 2.65 (1H), 3.06 (1H), 7.05 (1H), 7.32-7.43 (8H), 7.67 (1H), 7.77 (1H), 8.52 (1H).

### EXAMPLE 27c AND 27d

### (+)-2-{Hydroxy-3-[1-(2-fluoro-5-trifluoromethylphenyl)cyclopropyl]-2-phenyl)]-propionic acid}(3,4-dichlorophenyl)amide 27c and

### (−)-2-{hydroxy-3-[1-(2-fluoro-5-trifluoromethyl phenyl)-cyclopropyl]-2-phenylethynyl)]propionic acid}(3,4-dichlorophenyl)amide 27d

The racemic mixture obtained in Example 27b was separated into the enantiomers 27c and 27d by preparative chiral HPLC (Chiralpak AD 250×10 mm column).

27c: [α]^{D}₂₀=+15.4° (CHCl₃, 9.1 mg/1 ml; λ=589 nM)

27d: [α]^{D}₂₀=−15.9° (CHCl₃, 10.1 mg/l ml; λ=589 nM)

### EXAMPLE 28

### rac-{2-Hydroxy-3-[1-(2-fluoro-5-trifluoromethylphenyl)cyclopropyl]-2-(3-(1-piperidenyl)propynyl)]propionic acid}(4-cyano-3-trifluoromethylphenyl)amide

### 28a) rac-{2-Hydroxy-3-[1-(2-fluoro-5-trifluoromethylphenyl)cyclopropyl]-2-(3-bromopropynyl)]propionic acid}(4-cyano-3-trifluoromethylphenyl)amide

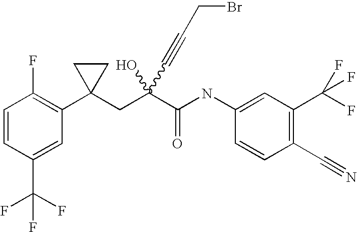

At −30° C., n-butyllithium (170 μl, 1.6 M in hexane) was added to a solution of 320 μl of diisopropylamine in tetrahydrofuran (5 ml). The mixture was stirred at this temperature for 30 minutes and cooled to −78° C. A solution of 3-bromopropyne (170 μl) in 4 ml of tetrahydrofuran was then added dropwise. The mixture was stirred at this temperature for 1 hour and then a solution of {3-[1-(2-fluoro-5-trifluoromethylphenyl)cyclopropyl]-2-oxopropionic acid}(4-cyano-3-trifluoromethylphenyl)amide (530 mg) in 4 ml of tetrahydrofuran was added dropwise. The mixture was then stirred at this temperature for about 3 h. The reaction mixture was subsequently poured into ice-cold saturated ammonium chloride solution. It was extracted with ethyl acetate. The combined organic phases were washed with saturated sodium chloride solution, dried over sodium sulphate and concentrated. The crude product was chromatographed on silica gel. 184 mg of product were obtained.

¹H-NMR (ppm, CDCl₃, 400 MHz): 0.83-0.88 (1H), 0.93-1.06 (3H), 2.28 (1H), 2.64 (1H), 2.99 (1H), 3.80 (2H), 7.07 (1H), 7.39 (1H), 7.59 (1H), 7.78 (2H), 7.90 (1H), 8.75 (1H).

### 28b) rac-{2-Hydroxy-3-[1-(2-fluoro-5-trifluoromethylphenyl)cyclopropyl]-2-(3-(1-piperidenyl)propynyl)]propionic acid}(4-cyano-3-trifluoromethylphenyl)amide

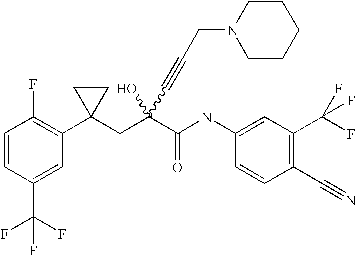

Piperidine (17 μl) was added to a suspension of the compound (50 mg) described in 28a) and potassium carbonate (24 mg) in dimethylformamide (2 ml). The mixture was stirred for 2 hours. The reaction mixture was diluted with ethyl acetate. The combined organic phases were washed with water and saturated sodium chloride solution, dried over sodium sulphate and concentrated. The crude product was chromatographed by preparative TLC. 37 mg of product were obtained.

¹H-NMR (ppm, CDCl₃, 400 MHz): 0.76-0.81 (1H), 0.89-1.02 (3H), 1.41 (2H), 1.57 (4H), 2.24 (1H), 2.42 (4H), 2.68 (1H), 3.15 (2H), 7.02 (1H), 7.34 (1H), 7.52 (1H), 7.77 (2H), 7.87 (1H), 8.95 (1H).

### EXAMPLE 29

### rac-{2-Hydroxy-3-[1-(2-fluoro-5-trifluoromethylphenyl)cyclopropyl]-2-(3-(4-methyl-1-piperazinyl)propyne)]propionic acid}(4-cyano-3-trifluoromethyl phenyl)amide

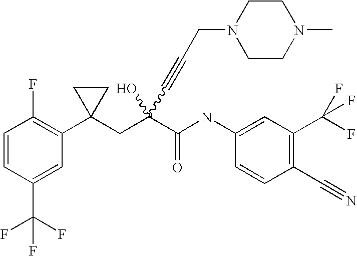

The compound described in Example 29 was prepared from rac-{2-hydroxy-3-[1-(2-fluoro-5-trifluoromethylphenyl)cyclopropyl]-2-(3-bromopropynyl)]propionic acid}(4-cyano-3-trifluoromethylphenyl)amide (see Example 28a) and 1-methylpiperazine in analogy to Example 28b).

¹H-NMR (ppm, CDCl₃, 400 MHz): 0.75-0.83 (1H), 0.90-1.03 (3H), 1.86 (4H), 2.24 (1H), 2.28 (3H), 2.55 (4H), 2.72 (1H), 3.26 (2H), 7.01 (1H), 7.32 (1H), 7.51 (1H), 7.78 (2H), 7.88 (1H), 8.95 (1H).

### EXAMPLE 30

### rac-{2-Hydroxy-3-[1-(2-fluoro-5-trifluoromethylphenyl)cyclopropyl]-2-(3-(4-carboxypiperidin-1-yl)propynyl)]-propionic acid}(4-cyano-3-trifluoromethylphenyl)amide

### 30a) rac-{2-Hydroxy-3-[1-(2-fluoro-5-trifluoromethylphenyl)cyclopropyl]-2-(3-(4-carboxy-methylpiperidin-1-yl)propynyl)]propionic acid}(4-cyano-3-trifluoromethylphenyl)amide

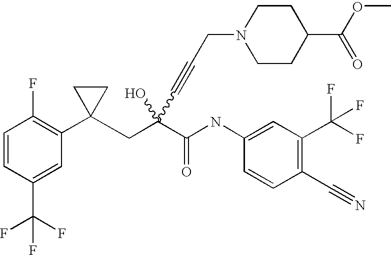

The compound described in Example 30a was prepared from rac-{2-hydroxy-3-[1-(2-fluoro-5-trifluoromethylphenyl)cyclopropyl]-2-(3-bromopropynyl)]propionic acid}-(4-cyano-3-trifluoromethylphenyl)amide and methyl piperidine-4-carboxylate in analogy to Example 28b).

¹H-NMR (ppm, CDCl₃, 400 MHz): 0.77-0.82 (1H), 0.91-1.02 (3H), 1.72-1.80 (2H), 1.91 (2H), 2.15 (2H), 2.23 (1H), 2.30-2.25 (1H), 2.70 (1H), 2.82 (2H), 3.19 (2H), 3.67 (3H), 7.02 (1H), 7.33 (1H), 7.52 (1H), 7.77 (2H), 7.88 (1H), 8.93 (1H).

### 30b) rac-{2-Hydroxy-3-[1-(2-fluoro-5-trifluoromethylphenyl)cyclopropyl]-2-(3-(4-carboxypiperidin-1-yl)propylnyl)]propionic acid}(4-cyano-3-trifluoromethylphenyl)amide

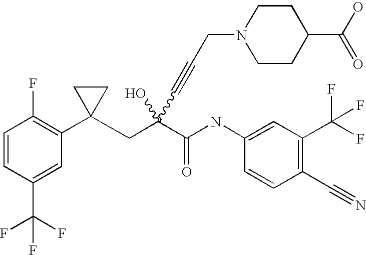

The compound described in Example 30b) was prepared from 30a) in analogy to Example 26b).

¹H-NMR (ppm, CDCl₃, 400 MHz): 0.76 (1H), 0.86 (1H), 0.94 (1H), 1.03 (1H), 1.72 (2H), 1.98 (2H), 2.15-2.26 (4H), 2.58 (1H), 3.15-3.29 (4H), 6.92 (1H), 7.25-7.28 (1H), 7.50 (1H), 7.73 (1H), 7.95 (2H), 9.71 (1H).

### EXAMPLE 31

### rac-2-{Hydroxy-3-[1-(2-fluoro-5-trifluoromethylphenyl)cyclopropyl]-2-phenylethynyl)]propionic acid}(5-indanyl)amide

### 31a) {3-[1-(2-fluoro-5-trifluoromethylphenyl)cyclopropyl]-2-oxopropionic acid}(5-indanyl)amide

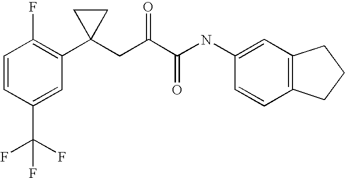

The compound described in Example 31a) was prepared from 3-[1-(2-fluoro-5-trifluoromethylphenyl)cyclopropyl]-2-oxopropionic acid and 5-aminoindane in analogy to the process described in Example 1a).

¹H-NMR (ppm, CDCl₃, 300 MHz): 0.99 (4H), 2.07 (2H), 2.88 (4H), 3.32 (2H), 7.09 (1H), 7.18 (1H), 7.25-7.28 (1H), 7.45-7.51 (2H), 7.73 (1H), 8.57 (1H).

### 31b) rac-2-{Hydroxy-3-[1-(2-fluoro-5-trifluoromethylphenyl)cyclopropyl]-2-phenylethynyl)]-propionic acid}(5-indanyl)amide

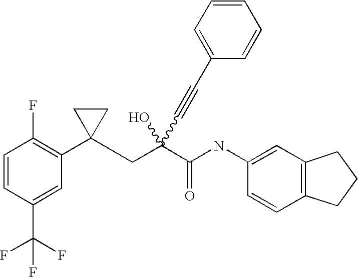

The compound described in Example 31b) was prepared from 31a) in analogy to Example 1b).

¹H-NMR (ppm, CDCl₃, 400 MHz): 0.84-1.08 (4H), 2.08 (2H), 2.45 (1H), 2.54 (1H), 2.89 (4H), 3.19 (1H), 6.99 (1H), 7.17 (2H), 7.28-7.34 (6H), 7.43 (1H), 7.64 (1H), 8.32 (1H).

### EXAMPLE 32

### rac-2-{Hydroxy-3-[1-(2-fluoro-5-trifluoromethylphenyl)cyclopropyl]-2-phenylethynyl)]-propionic acid}(3,4-dimethyl phenyl)amide

### 32a) {3-[1-(2-fluoro-5-trifluoromethylphenyl)cyclopropyl]-2-oxopropionic acid}(3,4-dimethylphenyl)amide

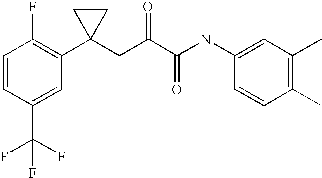

The compound described in Example 32a) was prepared from 3-[1-(2-fluoro-5-trifluoromethylphenyl)cyclopropyl]-2-oxopropionic acid and 3,4-dimethylaniline in analogy to the process described in Example 1a).

¹H-NMR (ppm, CDCl₃, 300 MHz): 0.99 (4H), 2.23 (3H), 2.25 (3H), 3.32 (2H), 7.06-7.11 (2H), 7.31 (1H), 7.36 (1H), 7.48 (1H), 7.73 (1H), 8.53 (1H).

32b) rac-{2-Hydroxy-3-[1-(2-fluoro-5-trifluoromethylphenyl)cyclopropyl]-2-phenylethynyl)]propionic acid}(3,4-dimethylphenyl)amide

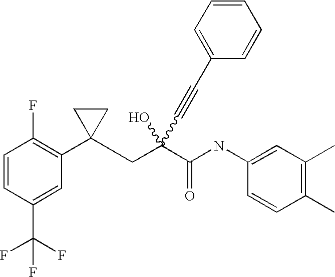

The compound described in Example 32b) was prepared from 32a) in analogy to Example 1b).

¹H-NMR (ppm, CDCl₃, 400 MHz): 0.86 (1H), 0.94 (1H), 0.98-1.05 (2H), 2.23 (3H), 2.25 (3H), 2.45 (1H), 2.53 (1H), 3.18 (1H), 6.99 (1H), 7.08 (1H), 7.23-7.33 (8H), 7.64 (1H), 8.28 (1H).

### EXAMPLE 33

### rac-2-{Hydroxy-3-[1-(2-fluoro-5-trifluoromethylphenyl)cyclopropyl]-2-phenylethynyl)]propionic acid}(6-quinolinyl)amide

### 33a) {3-[1-(2-Fluoro-5-trifluoromethylphenyl)cyclopropyl]-2-oxopropionic acid}(6-quinolinyl)amide

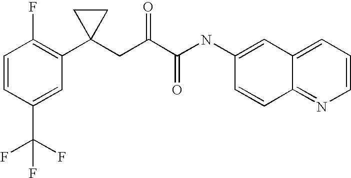

The compound described in Example 33a) was prepared from 3-[1-(2-fluoro-5-trifluoromethylphenyl)cyclopropyl]-2-oxopropionic acid and 6-aminoquinoline in analogy to the process described in Example 1a).

¹H-NMR (ppm, CDCl₃, 300 MHz): 1.02 (4H), 3.37 (2H), 7.10 (1H), 7.41 (1H), 7.49 (1H), 7.66 (1H), 7.75 (1H), 8.11 (2H), 8.37 (1H), 8.85-8.87 (2H).

### 33b) rac-{2-Hydroxy-3-[1-(2-fluoro-5-trifluoromethylphenyl)-cyclopropyl]-2-phenylethynyl)]propionic acid}(6-quinolinyl)amide

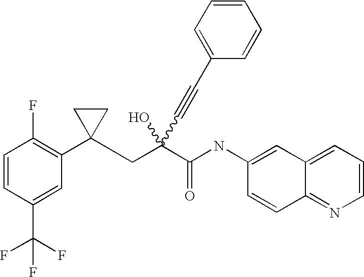

The compound described in Example 33b) was prepared from 33a) in analogy to Example 1b).

¹H-NMR (ppm, CDCl₃, 400 MHz): 0.86-1.09 (4H), 2.52 (1H), 2.66 (1H), 3.74 (1H), 6.97 (1H), 7.28-7.41 (7H), 7.56 (1H), 7.66 (1H), 8.05 (1H), 8.12 (1H), 8.29 (1H), 8.74 (1H), 8.83 (1H).

### EXAMPLE 34

### rac-{2-Hydroxy-3-[1-(2-fluoro-5-trifluoromethylphenyl)cyclopropyl]-2-(3-aminopropynyl)]propionic acid}(4-cyano-3-trifluoromethyl phenyl)amide

### 34a) rac-{2-Hydroxy-3-[1-(2-fluoro-5-trifluoromethylphenyl)-cyclopropyl]-2-(3-azidopropynyl)]propionic acid}(4-cyano-3-trifluoromethylphenyl)amide

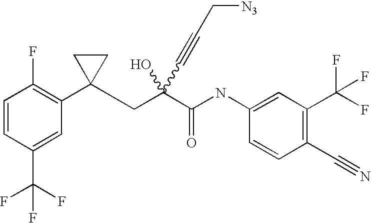

Sodium azide (28 mg) was added to a solution of the compound (130 mg) described in 28a) in dimethylformamide (2 ml). The mixture was stirred for 4 hours. The reaction mixture was diluted with ethyl acetate. The combined organic phases were washed with water and saturated sodium chloride solution, dried over sodium sulphate and concentrated. The crude product was chromatographed with silica gel. 86 mg of product were obtained.

¹H-NMR (ppm, CDCl₃, 400 MHz): 0.82-1.03 (4H), 2.32 (1H), 2.68 (1H), 3.12 (1H), 3.85 (2H), 7.06 (1H), 7.38 (1H), 7.56 (1H), 7.77 (2H), 7.88 (1H), 8.76 (1H).

### 34b) rac-{2-Hydroxy-3-[1-(2-fluoro-5-trifluoromethylphenyl)cyclopropyl]-2-(3-aminopropynyl)]propionic acid}(4-cyano-3-trifluoromethylphenyl)amide

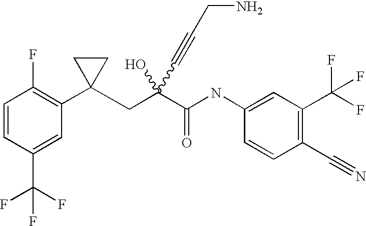

Triphenylphosphine (42 mg) was added to a solution of the compound (73 mg) described in 34a) in tetrahydrofuran (2 ml) and water (20 μl). The mixture was stirred for 7.5 hours. The reaction mixture was diluted with ethyl acetate. The combined organic phases were washed with saturated sodium bicarbonate solution and saturated sodium chloride solution, dried over sodium sulphate and concentrated. The crude product was chromatographed with silica gel. 12 mg of product were obtained.

¹H-NMR (ppm, CDCl₃, 400 MHz): 0.83 (1H), 0.92-1.00 (3H), 2.28 (1H), 2.60 (1H), 3.36 (2H), 7.04 (1H), 7.35 (1H), 7.53 (1H), 7.78 (2H), 7.91 (1H), 8.97 (1H).

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding U.S. Provisional Application Ser. No. 60/948,763, filed Jul. 10, 2007, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Compounds of the general formula I solved 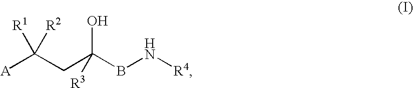
in which
R¹ and R² are independently of one another a hydrogen atom, a branched or unbranched C₁-C₅-alkyl group, further forming together with the C atom of the chain a ring having a total of 3-7 members,
R³ is a radical C≡C—R^{a}, where
R^{a} is a hydrogen or a C₁-C₈-alkyl, C₂-C₈-alkenyl, C₂-C₈-alkynyl, C₃-C₁₀-cycloalkyl, 3-8-membered heterocycloalkyl optionally substituted one or more times, identically or differently, by K, or C₆-C₁₂-aryl or 3-8-membered heteroaryl optionally substituted one or more times, identically or differently, by L, or silicon
K is a cyano, halogen, hydroxy, nitro, azido, —C(O)R^{b}, CO₂R^{b}, —O—R^{b}, —OSiR^{b}R^{c}R^{d}—S—R^{b}, SO₂NR^{c}R^{d}, —C(O)—NR^{c}R^{d}, —OC(O)—NR^{c}R^{d}, —C═NOR^{b}—NR^{c}R^{d} or C₃-C₁₀-cycloalkyl, 3-8-membered heterocycloalkyl optionally substituted one or more times, identically or differently, by M, or C₆-C₁₂-aryl or 3-8-membered heteroaryl optionally substituted one or more times, identically or differently, by L,
L is C₁-C₈-alkyl, C₂-C₈-alkenyl, C₂-C₈-alkynyl, C₁-C₆-perfluoroalkyl, C₁-C₆-perfluoroalkoxy, C₁-C₆-alkoxy-C₁-C₆-alkoxy, (CH₂)ₚ—C₃-C₁₀-cycloalkyl, (CH₂)ₚ-heterocycloalkyl, (CH₂)ₚCN, (CH₂)ₚHal, (CH₂)ₚNO₂, (CH₂)ₚ—C₆-C₁₂-aryl, (CH₂)ₚ-heteroaryl,
—(CH₂)ₚPO₃(R^{b})₂,
—(CH₂)ₚNR^{c}R^{d}, —(CH₂)ₚNR^{e}COR^{b},
—(CH₂)ₚNR^{e}CSR^{b}, —(CH₂)ₚNR^{e}S(O)R^{b},
—(CH₂)ₚNR^{e}S(O)₂R^{b}, —(CH₂)ₚNR^{e}CONR^{c}R^{d},
—(CH₂)ₚNR^{e}COOR^{b}, —(CH₂)ₚNR^{e}C(NH)NR^{c}R^{d},
—(CH₂)ₚNR^{e}CSNR^{c}R^{d}, —(CH₂)ₚNR^{e}S(O)NR^{c}R^{d},
—(CH₂)ₚNR^{e}S(O)₂NR^{c}R^{d}, —(CH₂)ₚCOR^{b},
—(CH₂)ₚCSR^{b}, —(CH₂)ₚS(O)R^{b},
—(CH₂)ₚS(O)(NH)R^{b}, —(CH₂)ₚS(O)₂R^{b},
—(CH₂)ₚS(O)₂NR^{c}R^{d}, —(CH₂)ₚSO₂OR^{b},
—(CH₂)ₚCO₂R^{b}, —(CH₂)ₚCONR^{c}R^{d},
—(CH₂)ₚCSNR^{c}R^{d}, —(CH₂)ₚOR^{b}, —(CH₂)ₚOCOR^{b}, —(CH₂)ₚSR^{b}, —(CH₂)ₚCR^{b}(OH)—R^{e}, —(CH₂)ₚ—C═NOR^{b}, —O—(CH₂)—O—, —O—(CH₂)ₙ—CH₂—, —O—CH═CH— or —(CH₂)ₙ₊₂—, where n is 1 or 2, and the terminal oxygen atoms and/or carbon atoms are linked to directly adjacent ring carbon atoms,
M is C₁-C₆-alkyl or a group —COR^{b}, CO₂R^{b}, —O—R^{b}, or —NR^{c}R^{b}, where
R^{b} is a hydrogen or a C₁-C₆-alkyl, C₂-C₈-alkenyl, C₂-C₈-alkynyl, C₃-C₁₀-cycloalkyl, C₆-C₁₂-aryl or C₁-C₃-perfluoroalkyl and
R^{c} and R^{d} are independently of one another a hydrogen, C₁-C₆-alkyl, C₂-C₈-alkenyl, C₂-C₈-alkynyl, C₃-C₁₀-cycloalkyl, C₆-C₁₂-aryl, C(O)R^{b} or a hydroxy group, where if
R^{c} is a hydroxy group, then R^{d} can only be a hydrogen, a C₁-C₆-alkyl, C₂-C₈-alkenyl, C₂-C₈-alkynyl, C₃-C₁₀-cycloalkyl or C₆-C₁₂-aryl and vice versa, and
R^{e} is a hydrogen, C₁-C₆-alkyl, C₂-C₈-alkenyl, C₂-C₈-alkynyl, C₃-C₁₀-cycloalkyl or C₆-C₁₂-aryl, and p can be an integral value from 0-6,
or
R³ is a radical C═C—R^{g}R^{h}, where
R^{g} and R^{h} are independently of one another a hydrogen or a C₁-C₈-alkyl, C₂-C₈-alkenyl or C₂-C₈-alkynyl optionally substituted one or more times, identically or differently, by X, in which
X is a cyano, halogen, hydroxy, nitro, —C(O)R^{b}, CO₂R^{b}, —O—R^{b}, —C(O)—NR^{c}R^{d}, —NR^{c}R^{d} with the meanings already mentioned before for R^{b}, R^{c} and R^{d}, and
R⁴ may be a 3-8-membered aromatic or heteroaromatic mono- or bicycle which is optionally substituted, identically or differently, by 1-3 radicals, or one of the following groups:
A: 6-membered/6-membered ring systems: 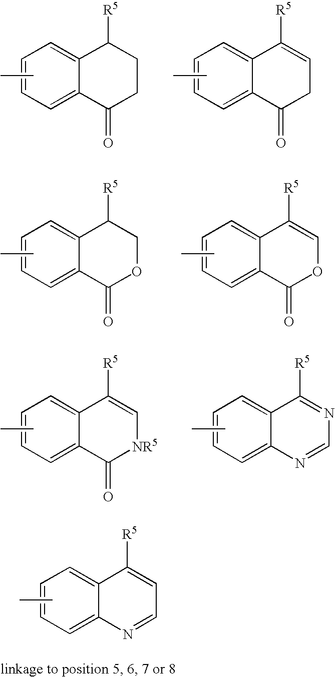
B: 6-membered/5-membered ring systems: 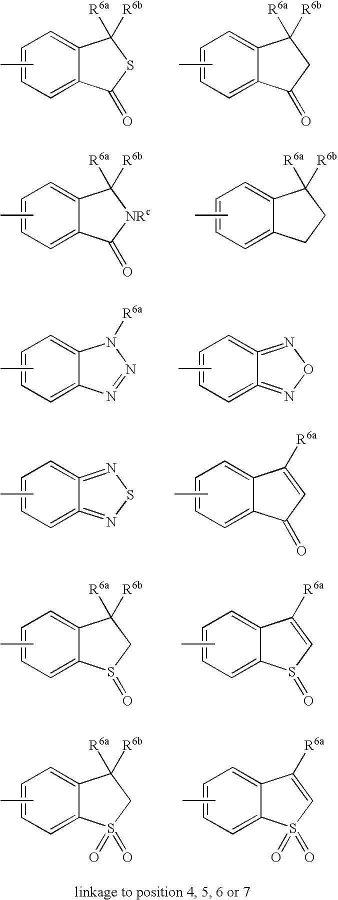
R⁵ may be hydrogen or C₁-C₄ alkyl or C₁-C₄ perfluoroalkyl,
R^{6a} and R^{6b} are independently of one another a hydrogen atom, a C₁-C₄-alkyl, a C₂-C₄-alkenyl or form together with the ring carbon atom a 3-6-membered ring,
A is a mono- or bicyclic carbocyclic or heterocyclic aromatic ring which may optionally be substituted one or more times, identically or differently, by C₁-C₈-alkyl, C₂-C₈-alkenyl, C₂-C₈-alkynyl, C₁-C₆-perfluoroalkyl, C₁-C₆-perfluoroalkoxy, C₁-C₆-alkoxy-C₁-C₆-alkyl, C₁-C₆-alkoxy-C₁-C₆-alkoxy, (CH₂)ₚ—C₃-C₁₀-cycloalkyl, (CH₂)ₚ-heterocycloalkyl, (CH₂)ₚCN, (CH₂)ₚHal, (CH₂)ₚNO₂, (CH₂)ₚ—C₆-C₁₂-aryl, (CH₂)ₚ-heteroaryl,
—(CH₂)ₚPO₃(R^{b})₂, —(CH₂)ₚNR^{c}R^{d}, —(CH₂)ₚNR^{e}COR^{b}, —(CH₂)ₚNR^{e}CS R^{b}, —(CH₂)ₚNR^{e}S(O)R^{b}, —(CH₂)ₚNR^{e}S(O)₂R^{b}, —(CH₂)ₚNR^{e}CONR^{c}R^{d}, (CH₂)ₚNR^{e}COOR^{b}, —(CH₂)ₚNR^{e}C(NH)NR^{c}R^{d}, —(CH₂)ₚNR^{e}CSNR^{c}R^{d}, —(CH₂)ₚNR^{e}S(O)NR^{c}R^{d}, —(CH₂)ₚNR^{e}S(O)₂NR^{c}R^{d}, —(CH₂)ₚCOR^{b}, —(CH₂)ₚCSR^{b}, —(CH₂)ₚS(O)R^{b}, —(CH₂)ₚS(O)(NH)R^{b}, —(CH₂)ₚS(O)₂R^{b}, —(CH₂)ₚS(O)₂NR^{c}R^{d}, —(CH₂)ₚSO₂OR^{b}, —(CH₂)ₚCO₂R^{e}, —(CH₂)ₚCONR^{c}R^{d}, —(CH₂)ₚCSNR^{c}R^{d}, —(CH₂)ₚOR^{b}, —(CH₂)ₚSR^{b}, —(CH₂)ₚCR^{b}(OH)—R^{d}, —(CH₂)ₚ—C═NOR^{b}, —O— (CH₂)ₙ—O—, —O—(CH₂)ₙ—CH₂—, —O—CH═CH— or —(CH₂)ₙ₊₂—, where n is 1 or 2, and the terminal oxygen atoms and/or carbon atoms are linked to directly adjacent ring carbon atoms, or
A is a radical —CO₂R^{b}, C(O)NR^{c}R^{d}, COR^{b},
or
A is an alkenyl group —CR⁵═CR⁶R⁷, where
R⁵, R⁶ and R⁷ are identical or different and are independently of one another hydrogen atoms, halogen atoms, aryl radicals or an unsubstituted or partly or completely fluorinated C₁-C₅-alkyl group, or
A is an alkynyl group —C≡CR⁵, with the meaning stated above for R⁵, and
B is a carbonyl or a CH₂ group,
and their pharmaceutically acceptable salts.

2. Compounds according to claim 1, in which R¹ and R² are each independently of one another a hydrogen atom, a methyl or ethyl radical, or form together with the C atom of the chain a ring having a total of 3-7 members.

3. Compounds according to claim 2, in which R¹ and R² are preferably simultaneously a hydrogen atom, a methyl or cyclopropyl radical, particularly preferably a methyl or cyclopropyl radical.

4. Compounds according to claim 1, in which R³ is an alkynyl radical of the formula radical C≡C—R^{a} with
R^{a} a C₁-C₄-alkyl, C₃-C₁₀-cycloalkyl, 3-8-membered heterocycloalkyl which is optionally substituted by K, or optionally a C₆-C₁₂-aryl or 3-8-membered heteroaryl which is substituted by L,
K a cyano, halogen, hydroxy, —O—R^{b}, SO₂NR^{c}R^{d}, —C(O)—NR^{c}R^{d}, —NR^{c}R^{d} or a 3-8-membered heterocycloalkyl radical which is optionally substituted one or more times, identically or differently, by M, or an aryl or heteroaryl which is optionally substituted more than once, identically or differently, by L, and
L a C₁-C₄-alkyl, C₁-C₄-perfluoroalkyl, (CH₂)ₙ—C₃-C₁₀-cycloalkyl, (CH₂)ₚ-heterocycloalkyl, (CH₂)ₚCN, (CH₂)ₚHal, (CH₂)ₚNO₂, (CH₂)ₚ—C₆-C₁₂-aryl, (CH₂)ₚ-heteroaryl, —(CH₂)ₚNR^{c}R^{d}, —(CH₂)ₚNR^{e}S(O)₂R^{b}, —(CH₂)ₚS(O)₂NR^{c}R^{d}, —(CH₂)ₚCONR^{c}R^{d}, (CH₂)ₚOR^{b}, —(CH₂)ₚOCOR^{b}, —(CH₂)ₚCR^{b}(OH)—R^{e}, —(CH₂)ₚCO₂R^{b},
M a C₁-C₄-alkyl or a group —CO₂R^{b}, —O—R^{b} or —NR^{c}R^{d}, where
R^{b} is a hydrogen or a C₁-C₆-alkyl, C₃-C₁₀-cycloalkyl, C₆-C₁₂-aryl or C₁-C₃-perfluoroalkyl and
R^{c} and R^{d} are independently of one another a hydrogen, a C₁-C₆-alkyl, C₃-C₁₀-cycloalkyl, C₆-C₁₂-aryl, C(O)R^{b} or a hydroxy group, where if
R^{c} is a hydroxy group, then
R^{d} can only be a hydrogen, a C₁-C₆-alkyl, C₂-C₈-alkenyl, C₂-C₈-alkynyl, C₃-C₁₀-cycloalkyl or C₆-C₁₂-aryl, and vice versa, and
R^{e} is a hydrogen, C₁-C₆-alkyl or C₆-C₁₂-aryl, and
p may be a number 0, 1, 2 or 3.

5. Compounds according to claim 4, in which R^{a} is a C₁-C₄-alkyl radical which is optionally substituted by K, a phenyl or hetaryl radical which is optionally substituted by L.

6. Compounds according to claim 5, in which L is a methyl, trifluoromethyl, methoxy, acetoxy, hydroxy, carboxyl or carboxyalkyl radical.

7. Compounds according to claim 1, in which R⁴ is preferably a phenyl ring, particularly preferably a phenyl ring substituted identically or differently by 1-3 radicals.

8. Compounds according to claim 7, in which the phenyl ring is preferably substituted by nitro, trifluoromethyl, pentafluoroethyl, cyano, chlorine, fluorine, methyl.

9. Compounds according to claim 1, in which R⁴ is preferably one of the following groups
A: 6-membered/6-membered ring systems: 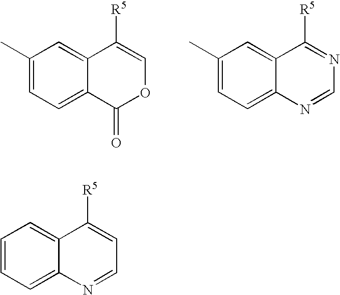
or
B: 6-membered/5-membered ring systems: 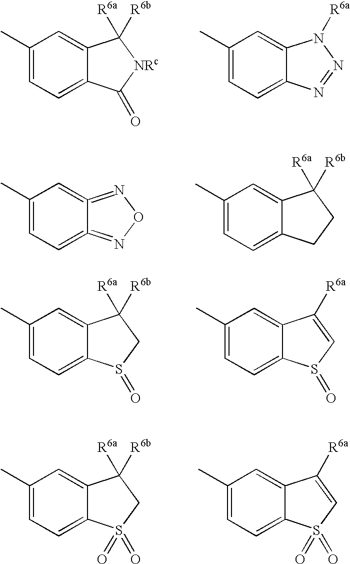
in which R⁵ and R^{6a} and R^{6b} have the meanings mentioned in claim 1.

10. Compounds according to claim 1, in which A can preferably be substituted by the following radicals: C₁-C₈-alkyl, C₁-C₆-perfluoroalkyl, C₁-C₆-perfluoroalkoxy, C₁-C₆-alkoxy-C₁-C₆-alkyl, C₁-C₆-alkoxy-C₁-C₆-alkoxy, (CH₂)ₙ—C₃-C₁₀-cycloalkyl, (CH₂)ₚ-heterocycloalkyl, (CH₂)ₚCN, (CH₂)ₚHal, (CH₂)ₚNO₂, (CH₂)ₚ—C₆-C₁₂-aryl, (CH₂)ₚ-heteroaryl, —(CH₂)ₚNR^{c}R^{d}, —(CH₂)ₚNR^{e}COR^{b}, —(CH₂)ₚNR^{e}S(O)₂R^{b}, —(CH₂)ₚNR^{e}CONR^{c}R^{d}, —(CH₂)ₚNR^{e}S(O)₂NR^{c}R^{d}, (CH₂)ₚCOR^{b}, —(CH₂)ₚCSR^{b}, —(CH₂)ₚS(O)(NH)R^{b}, —(CH₂)ₚS(O)₂R^{b}, —(CH₂)ₚS(O)₂NR^{c}R^{d}, —(CH₂)ₚCO₂R^{b}, —(CH₂)ₚCONR^{c}R^{d}, —(CH₂)ₚOR^{b}, —(CH₂)ₚSR^{b}, —(CH₂)ₚCR^{b}(OH)—R^{d}, —(CH₂)ₚ—C═NOR^{b}, —O—(CH₂)ₙ—O—, —O—(CH₂)ₙ—CH₂—, —O—CH═CH— or —(CH₂)ₙ₊₂—, where n=1 or 2 and the terminal oxygen atoms and/or carbon atoms are linked to directly adjacent ring carbon atoms.

11. Compounds according to claim 10, in which A is particularly preferably substituted by C₁-C₄-alkyl, C₁-C₂-perfluoroalkyl, C₁-C₂-perfluoroalkoxy, (CH₂)ₚCN, (CH₂)ₚHal, —(CH₂)ₚNR^{c}R^{d}, —(CH₂)ₚS(O)(NH)R^{b}, —(CH₂)ₚS(O)₂R^{b}, (CH₂)ₚS(O)₂NR^{c}R^{d}, —(CH₂)ₚOR^{b} or —(CH₂)ₚSR^{b}, and p and R^{b}, R^{c} and R^{d}.

12. Compounds according to claim 1, in which A is preferably an unsubstituted phenyl ring.

13. Compounds according to claim 11, in which A is preferably a phenyl ring substituted once or twice, identically or differently, by fluorine, chlorine, bromine, methyl, trifluoromethyl or methoxy.

14. Compounds according to claim 1, in which B is a carbonyl group.

15. Compounds according to claim 1, in which B is a —CH₂— group.

16. Compounds according to claim 1, in which p is preferably 0 or 1.

17. Compounds according to any of claim 1, specifically the compounds mentioned below, and the use thereof are preferred according to the invention:

| | | |
|---|---|---|
| | Racemic or | |
| No. | enantiomer | R3 |

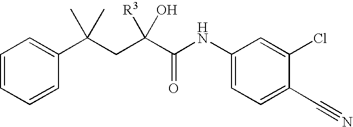
| | | |
|---|---|---|
| 1 2 3 | rac+− | 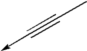 |
| 4 5 6 | rac+− | 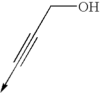 |
| 7 8 9 | rac+− | 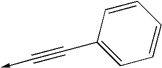 |
| 10 11 12 | rac+− | 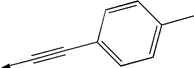 |
| 13 14 15 | rac+− | 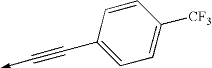 |
| 16 17 18 | rac+− | 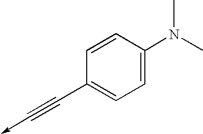 |
| 19 20 21 | rac+− | 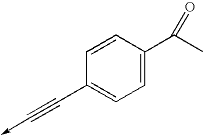 |
| 22 23 24 | rac+− | 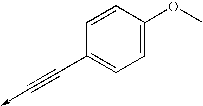 |
| 25 26 27 | rac+− | 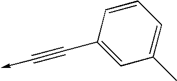 |
| 28 29 30 | rac+− | 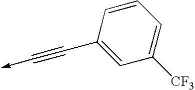 |
| 31 32 33 | rac+− | 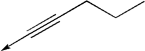 |
| 34 35 36 | rac+− | 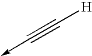 |
| 37 38 39 | rac+− | 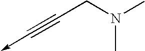 |
| 40 41 42 | rac+− | 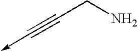 |
| 43 44 45 | rac+− | 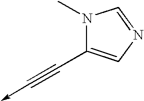 |
| 46 47 48 | rac+− | 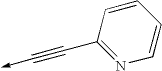 |
| 49 50 51 | rac+− | 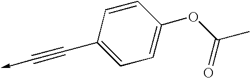 |
| 52 53 54 | rac+− | 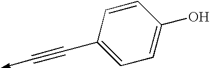 |
| 55 56 57 | rac+− | 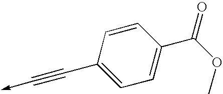 |
| 58 59 60 | rac+− | 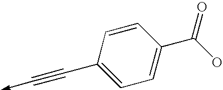 |
| 61 62 63 | rac+− | 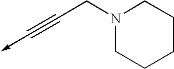 |
| 64 65 66 | rac+− | 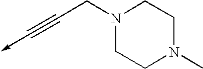 |
| 67 68 69 | rac+− | 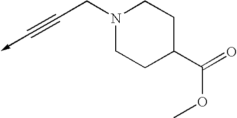 |
| 70 71 72 | rac+− | 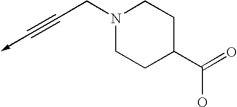 |
| 73 74 75 | rac+− | 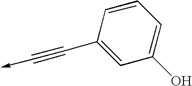 |
| 76 77 78 | rac+− | 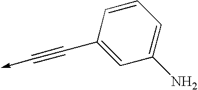 |
| 79 80 81 | rac+− | 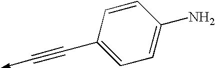 |
| 82 83 84 | rac+− | 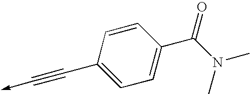 |
| 85 86 87 | rac+− | 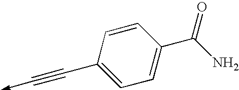 |

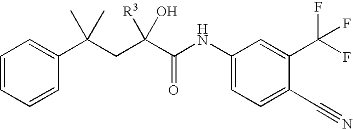
| | | |
|---|---|---|
| 88 89 90 | rac+− | 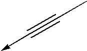 |
| 91 92 93 | rac+− | 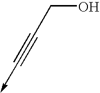 |
| 94 95 96 | rac+− | 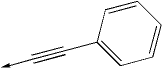 |
| 97 98 99 | rac+− | 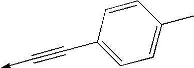 |
| 100 101 102 | rac+− | 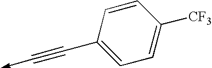 |
| 103 104 105 | rac+− | 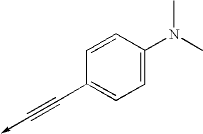 |
| 106 107 108 | rac+− | 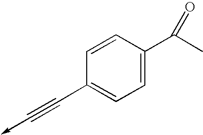 |
| 109 110 111 | rac+− | 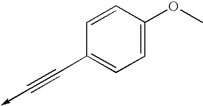 |
| 112 113 114 | rac+− | 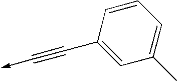 |
| 115 116 117 | rac+− | 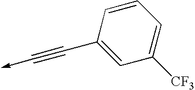 |
| 118 119 120 | rac+− | 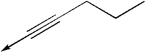 |
| 121 122 123 | rac+− | 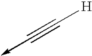 |
| 124 125 126 | rac+− | 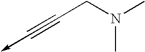 |
| 127 128 129 | rac+− | 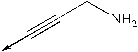 |
| 130 131 132 | rac+− | 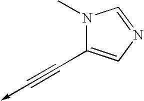 |
| 133 134 135 | rac+− | 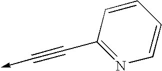 |
| 136 137 138 | rac+− | 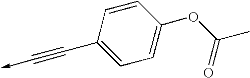 |
| 139 140 141 | rac+− | 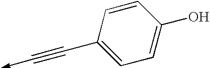 |
| 142 143 144 | rac+− | 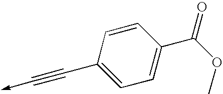 |
| 145 146 147 | rac+− | 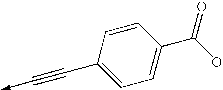 |
| 148 149 150 | rac+− | 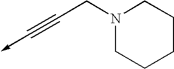 |
| 151 152 153 | rac+− | 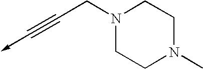 |
| 154 155 156 | rac+− | 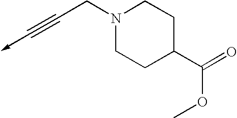 |
| 157 158 159 | rac+− | 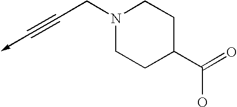 |
| 160 161 162 | rac+− | 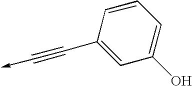 |
| 163 164 165 | rac+− | 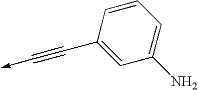 |
| 166 167 168 | rac+− | 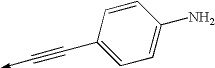 |
| 169 170 171 | rac+− | 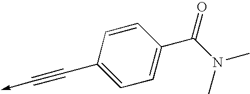 |
| 172 173 174 | rac+− | 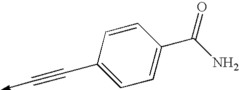 |

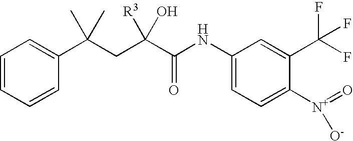
| | | |
|---|---|---|
| 175 176 177 | rac+− | 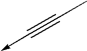 |
| 178 179 180 | rac+− | 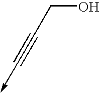 |
| 181 182 183 | rac+− | 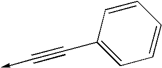 |
| 184 185 186 | rac+− | 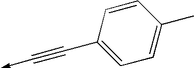 |
| 187 188 189 | rac+− | 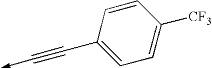 |
| 190 191 192 | rac+− | 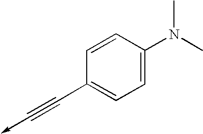 |
| 193 194 195 | rac+− | 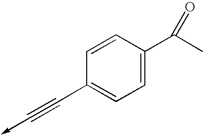 |
| 196 197 198 | rac+− | 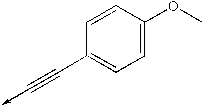 |
| 199 200 201 | rac+− | 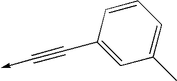 |
| 202 203 204 | rac+− | 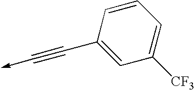 |
| 205 206 207 | rac+− | 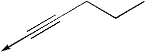 |
| 208 209 210 | rac+− | 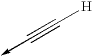 |
| 211 212 213 | rac+− | 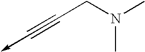 |
| 214 215 216 | rac+− | 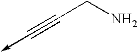 |
| 217 218 219 | rac+− | 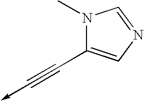 |
| 220 221 222 | rac+− | 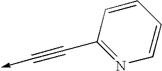 |
| 223 224 225 | rac+− | 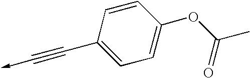 |
| 226 227 228 | rac+− | 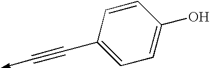 |
| 229 230 231 | rac+− | 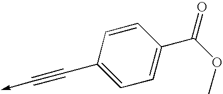 |
| 232 233 234 | rac+− | 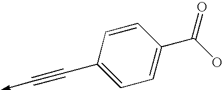 |
| 235 236 237 | rac+− | 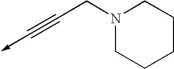 |
| 238 239 240 | rac+− | 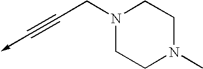 |
| 241 242 243 | rac+− | 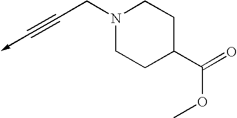 |
| 244 245 246 | rac+− | 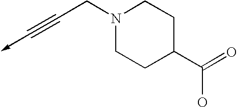 |
| 247 248 249 | rac+− | 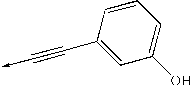 |
| 250 251 252 | rac+− | 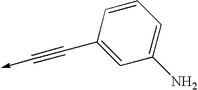 |
| 253 254 255 | rac+− | 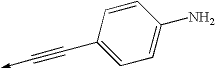 |
| 256 257 258 | rac+− | 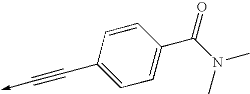 |
| 259 260 261 | rac+− | 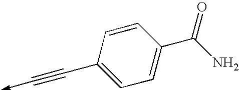 |

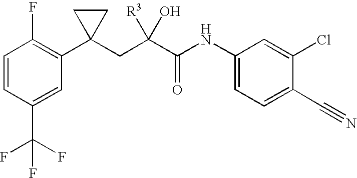
| | | |
|---|---|---|
| 262 263 264 | rac+− | 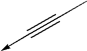 |
| 265 266 267 | rac+− | 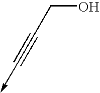 |
| 268 269 270 | rac+− | 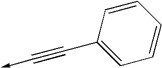 |
| 271 272 273 | rac+− | 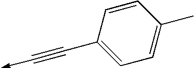 |
| 274 275 276 | rac+− | 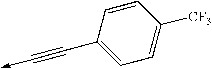 |
| 277 278 279 | rac+− | 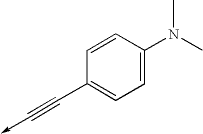 |
| 280 281 282 | rac+− | 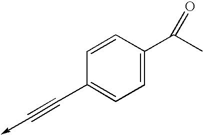 |
| 283 284 285 | rac+− | 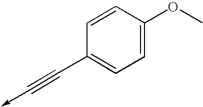 |
| 286 287 288 | rac+− | 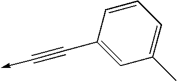 |
| 289 290 291 | rac+− | 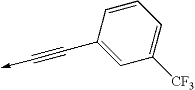 |
| 292 293 294 | rac+− | 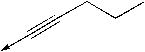 |
| 295 296 297 | rac+− | 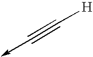 |
| 298 299 300 | rac+− | 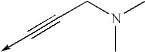 |
| 301 302 303 | rac+− | 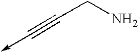 |
| 304 305 306 | rac+− | 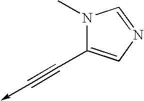 |
| 307 308 309 | rac+− | 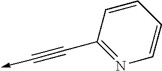 |
| 310 311 312 | rac+− | 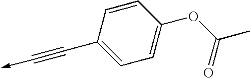 |
| 313 314 315 | rac+− | 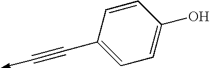 |
| 316 317 318 | rac+− | 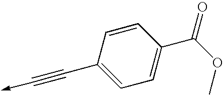 |
| 319 320 321 | rac+− | 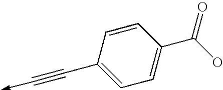 |
| 322 323 324 | rac+− | 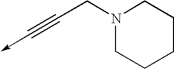 |
| 325 326 327 | rac+− | 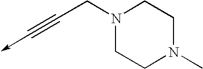 |
| 328 329 330 | rac+− | 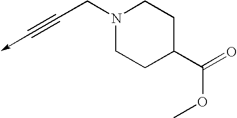 |
| 331 332 333 | rac+− | 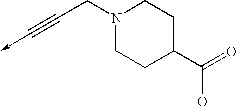 |
| 334 335 336 | rac+− | 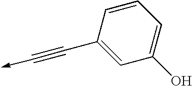 |
| 337 338 339 | rac+− | 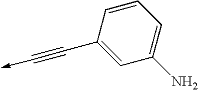 |
| 340 341 342 | rac+− | 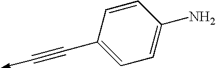 |
| 343 344 345 | rac+− | 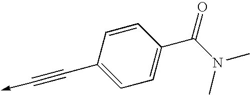 |
| 346 347 348 | rac+− | 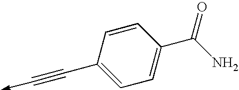 |

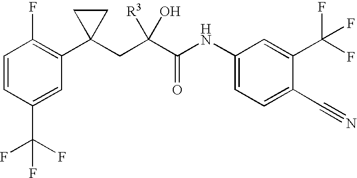
| | | |
|---|---|---|
| 349 350 351 | rac+− | 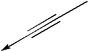 |
| 352 353 354 | rac+− | 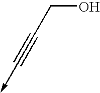 |
| 355 356 357 | rac+− | 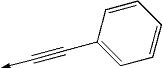 |
| 358 359 360 | rac+− | 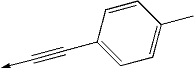 |
| 361 362 363 | rac+− | 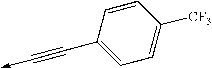 |
| 364 365 366 | rac+− | 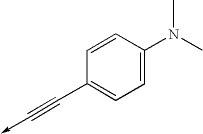 |
| 367 368 369 | rac+− | 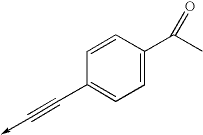 |
| 370 371 372 | rac+− | 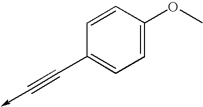 |
| 373 374 375 | rac+− | 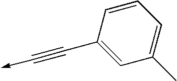 |
| 376 377 378 | rac+− | 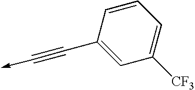 |
| 379 380 381 | rac+− | 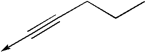 |
| 382 383 384 | rac+− | 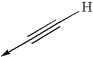 |
| 385 386 387 | rac+− | 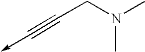 |
| 388 389 390 | rac+− | 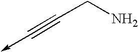 |
| 391 392 393 | rac+− | 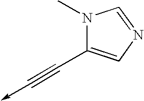 |
| 394 395 396 | rac+− | 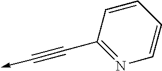 |
| 397 398 399 | rac+− | 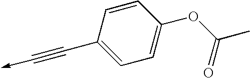 |
| 400 401 402 | rac+− | 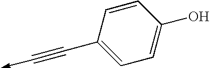 |
| 403 404 405 | rac+− | 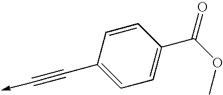 |
| 406 407 408 | rac+− | 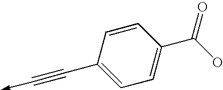 |
| 409 410 411 | rac+− | 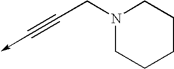 |
| 412 413 414 | rac+− | 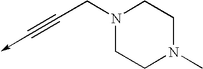 |
| 415 416 417 | rac+− | 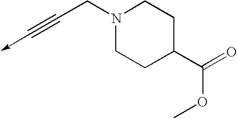 |
| 418 419 420 | rac+− | 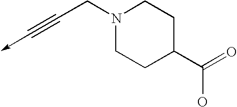 |
| 421 422 423 | rac+− | 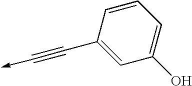 |
| 424 425 426 | rac+− | 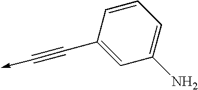 |
| 427 428 429 | rac+− | 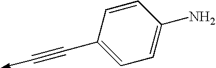 |
| 430 431 432 | rac+− | 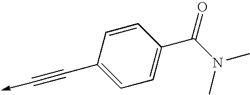 |
| 433 434 435 | rac+− | 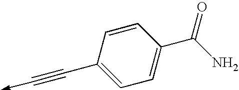 |

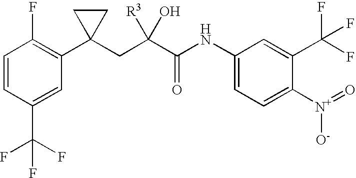
| | | |
|---|---|---|
| 436 437 438 | rac+− | 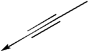 |
| 439 440 441 | rac+− | 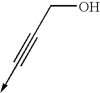 |
| 442 443 444 | rac+− | 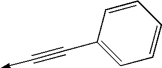 |
| 445 446 447 | rac+− | 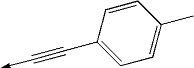 |
| 448 449 450 | rac+− | 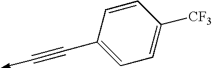 |
| 451 452 453 | rac+− | 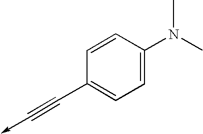 |
| 454 455 456 | rac+− | 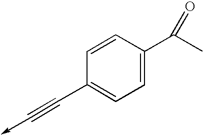 |
| 457 458 459 | rac+− | 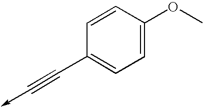 |
| 460 461 462 | rac+− | 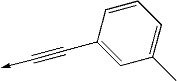 |
| 463 464 465 | rac+− | 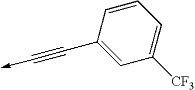 |
| 466 467 468 | rac+− | 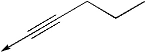 |
| 469 470 471 | rac+− | 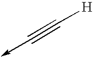 |
| 472 473 474 | rac+− | 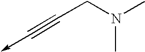 |
| 475 476 477 | rac+− | 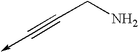 |
| 478 479 480 | rac+− | 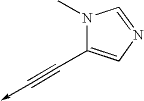 |
| 481 482 483 | rac+− | 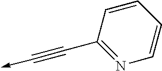 |
| 484 485 486 | rac+− | 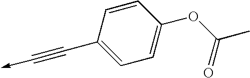 |
| 487 488 489 | rac+− | 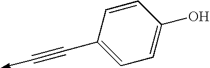 |
| 490 491 492 | rac+− | 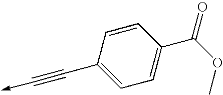 |
| 493 494 495 | rac+− | 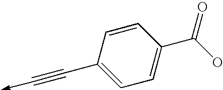 |
| 496 497 498 | rac+− | 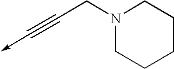 |
| 499 500 501 | rac+− | 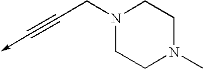 |
| 502 503 504 | rac+− | 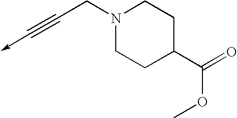 |
| 505 506 507 | rac+− | 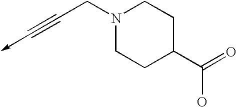 |
| 508 509 510 | rac+− | 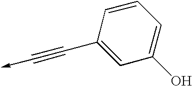 |
| 511 512 513 | rac+− | 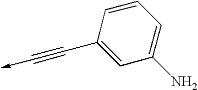 |
| 514 515 516 | rac+− | 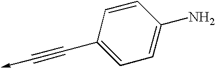 |
| 517 518 519 | rac+− | 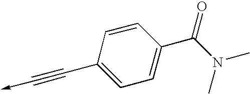 |
| 520 521 522 | rac+− | 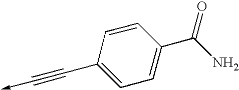 |

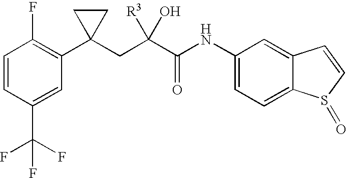
| | | |
|---|---|---|
| 523 524 525 | rac+− | 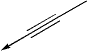 |
| 526 527 528 | rac+− | 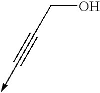 |
| 529 530 531 | rac+− | 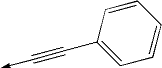 |
| 532 533 534 | rac+− | 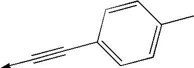 |
| 535 536 537 | rac+− | 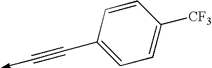 |
| 538 539 540 | rac+− | 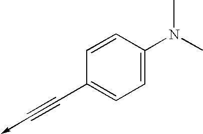 |
| 541 542 543 | rac+− | 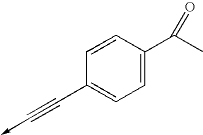 |
| 544 545 546 | rac+− | 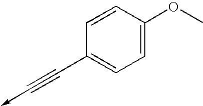 |
| 547 548 549 | rac+− | 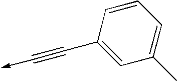 |
| 550 551 552 | rac+− | 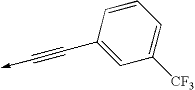 |
| 553 554 555 | rac+− | 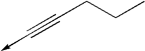 |
| 556 557 558 | rac+− | 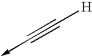 |
| 559 560 561 | rac+− | 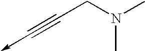 |
| 562 563 564 | rac+− | 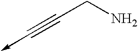 |
| 565 566 567 | rac+− | 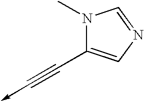 |
| 568 569 570 | rac+− | 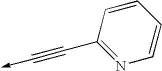 |
| 571 572 573 | rac+− | 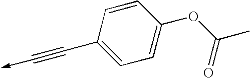 |
| 574 575 576 | rac+− | 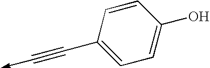 |
| 577 578 579 | rac+− | 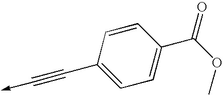 |
| 580 581 582 | rac+− | 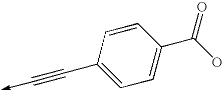 |
| 583 584 585 | rac+− | 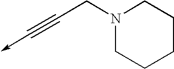 |
| 586 587 588 | rac+− | 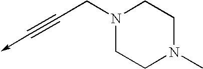 |
| 589 590 591 | rac+− | 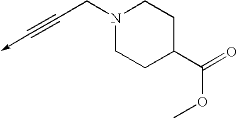 |
| 592 593 594 | rac+− | 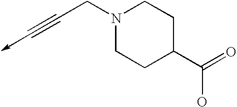 |
| 595 596 597 | rac+− | 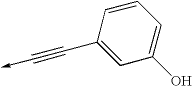 |
| 598 599 600 | rac+− | 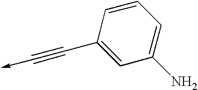 |
| 601 602 603 | rac+− | 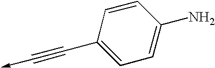 |
| 604 605 606 | rac+− | 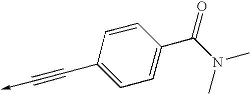 |
| 607 608 609 | rac+− | 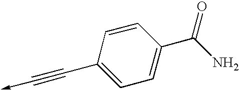 |

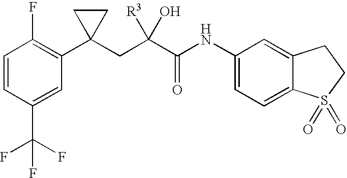
| | | |
|---|---|---|
| 610 611 612 | rac+− | 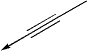 |
| 613 614 615 | rac+− | 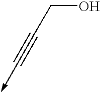 |
| 616 617 618 | rac+− | 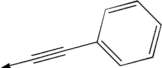 |
| 619 620 621 | rac+− | 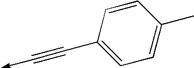 |
| 622 623 624 | rac+− | 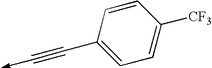 |
| 625 626 627 | rac+− | 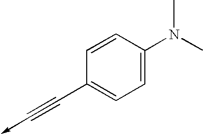 |
| 628 629 630 | rac+− | 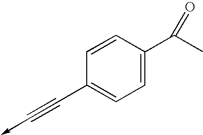 |
| 631 632 633 | rac+− | 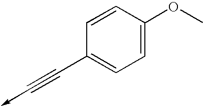 |
| 634 635 636 | rac+− | 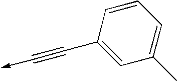 |
| 637 638 639 | rac+− | 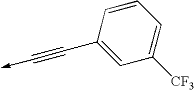 |
| 640 641 642 | rac+− | 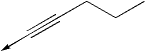 |
| 643 644 645 | rac+− | 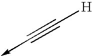 |
| 646 647 648 | rac+− | 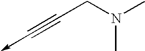 |
| 649 650 651 | rac+− | 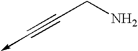 |
| 652 653 654 | rac+− | 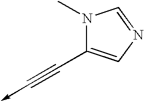 |
| 655 656 657 | rac+− | 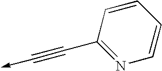 |
| 658 659 660 | rac+− | 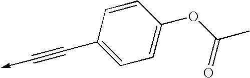 |
| 661 662 663 | rac+− | 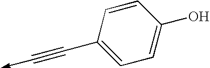 |
| 664 665 666 | rac+− | 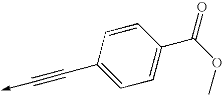 |
| 667 668 669 | rac+− | 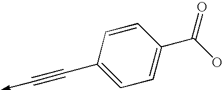 |
| 670 671 672 | rac+− | 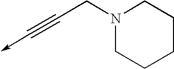 |
| 673 674 675 | rac+− | 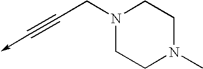 |
| 676 677 678 | rac+− | 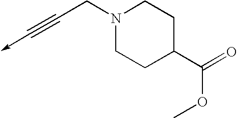 |
| 679 680 681 | rac+− | 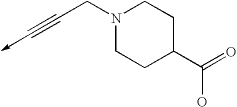 |
| 682 683 684 | rac+− | 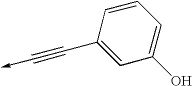 |
| 685 686 687 | rac+− | 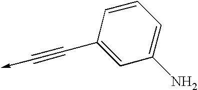 |
| 688 689 690 | rac+− | 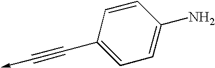 |
| 691 692 693 | rac+− | 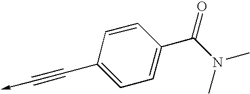 |
| 694 695 696 | rac+− | 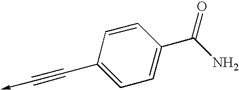 |

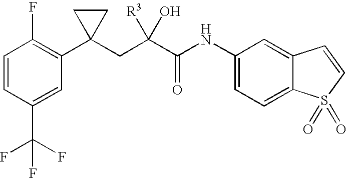
| | | |
|---|---|---|
| 697 698 699 | rac+− | 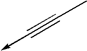 |
| 700 701 702 | rac+− | 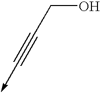 |
| 703 704 705 | rac+− | 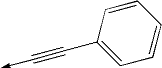 |
| 706 707 708 | rac+− | 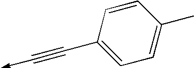 |
| 709 710 711 | rac+− | 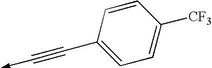 |
| 712 713 714 | rac+− | 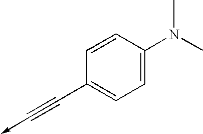 |
| 715 716 717 | rac+− | 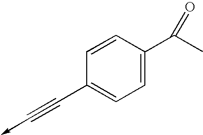 |
| 718 719 720 | rac+− | 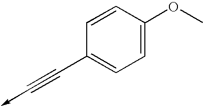 |
| 721 722 723 | rac+− | 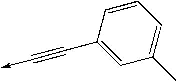 |
| 724 725 726 | rac+− | 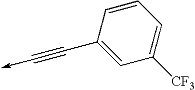 |
| 727 728 729 | rac+− | 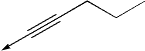 |
| 730 731 732 | rac+− | 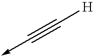 |
| 733 734 735 | rac+− | 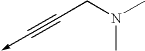 |
| 736 737 738 | rac+− | 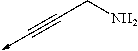 |
| 739 740 741 | rac+− | 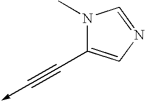 |
| 742 743 744 | rac+− | 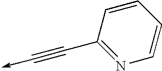 |
| 745 746 747 | rac+− | 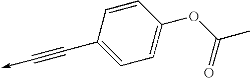 |
| 748 749 750 | rac+− | 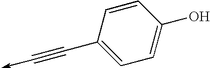 |
| 751 752 753 | rac+− | 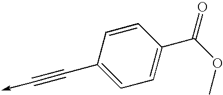 |
| 754 755 756 | rac+− | 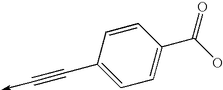 |
| 757 758 759 | rac+− | 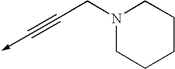 |
| 760 761 762 | rac+− | 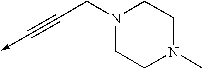 |
| 763 764 765 | rac+− | 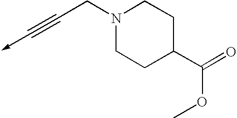 |
| 766 767 768 | rac+− | 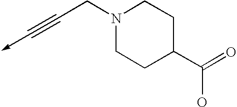 |
| 769 770 771 | rac+− | 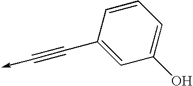 |
| 772 773 774 | rac+− | 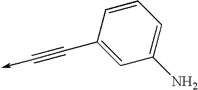 |
| 775 776 777 | rac+− | 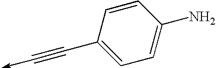 |
| 778 779 780 | rac+− | 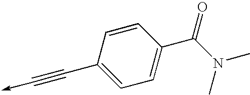 |
| 781 782 783 | rac+− | 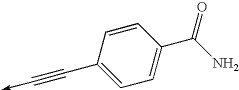 |

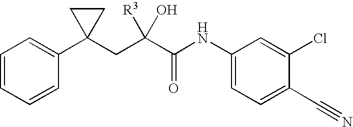
| | | |
|---|---|---|
| 784 785 786 | rac+− | 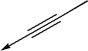 |
| 787 788 789 | rac+− | 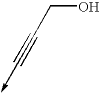 |
| 790 791 792 | rac+− | 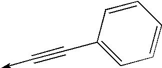 |
| 793 794 795 | rac+− | 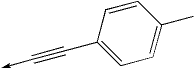 |
| 796 797 798 | rac+− | 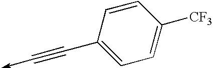 |
| 799 800 801 | rac+− | 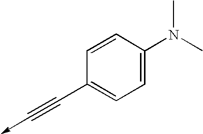 |
| 802 803 804 | rac+− | 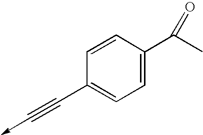 |
| 805 806 807 | rac+− | 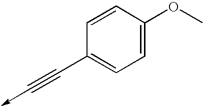 |
| 808 809 810 | rac+− | 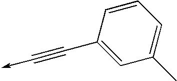 |
| 811 812 813 | rac+− | 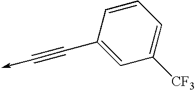 |
| 814 815 816 | rac+− | 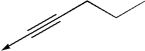 |
| 817 818 819 | rac+− | 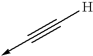 |
| 820 821 822 | rac+− | 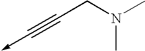 |
| 823 824 825 | rac+− | 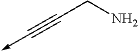 |
| 826 827 828 | rac+− | 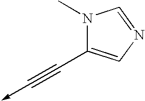 |
| 829 830 831 | rac+− | 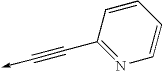 |
| 832 833 834 | rac+− | 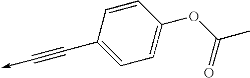 |
| 835 836 837 | rac+− | 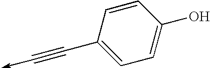 |
| 838 839 840 | rac+− | 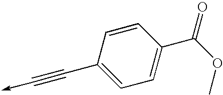 |
| 841 842 843 | rac+− | 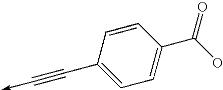 |
| 844 845 846 | rac+− | 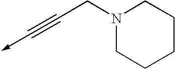 |
| 847 848 849 | rac+− | 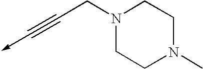 |
| 850 851 852 | rac+− | 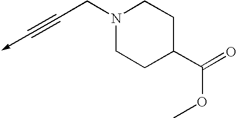 |
| 853 854 855 | rac+− | 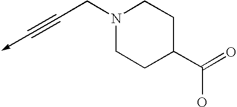 |
| 856 857 858 | rac+− | 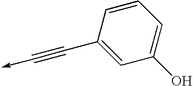 |
| 859 860 861 | rac+− | 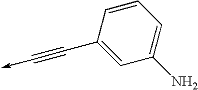 |
| 862 863 864 | rac+− | 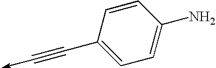 |
| 865 866 867 | rac+− | 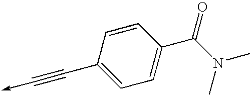 |
| 868 869 870 | rac+− | 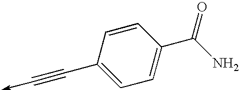 |

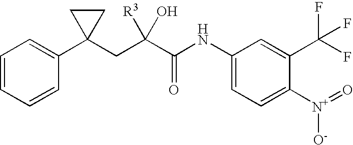
| | | |
|---|---|---|
| 871 872 873 | rac+− | 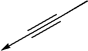 |
| 874 875 876 | rac+− | 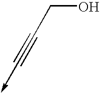 |
| 877 878 879 | rac+− | 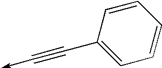 |
| 880 881 882 | rac+− | 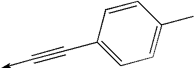 |
| 883 884 885 | rac+− | 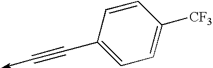 |
| 886 887 888 | rac+− | 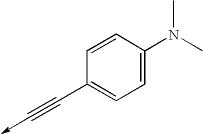 |
| 889 890 891 | rac+− | 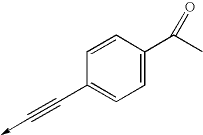 |
| 892 893 894 | rac+− | 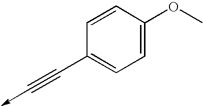 |
| 895 896 897 | rac+− | 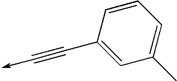 |
| 898 899 900 | rac+− | 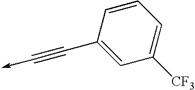 |
| 901 902 903 | rac+− | 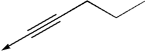 |
| 904 905 906 | rac+− | 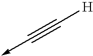 |
| 907 908 909 | rac+− | 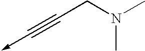 |
| 910 911 912 | rac+− | 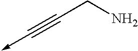 |
| 913 914 915 | rac+− | 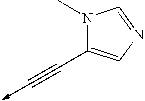 |
| 916 917 918 | rac+− | 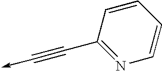 |
| 919 920 921 | rac+− | 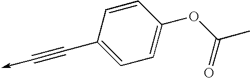 |
| 922 923 924 | rac+− | 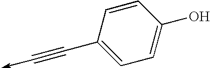 |
| 925 926 927 | rac+− | 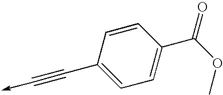 |
| 928 929 930 | rac+− | 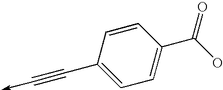 |
| 931 932 933 | rac+− | 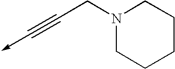 |
| 934 935 936 | rac+− | 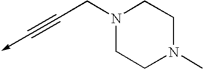 |
| 937 938 939 | rac+− | 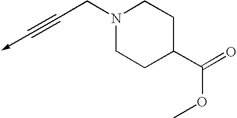 |
| 940 941 942 | rac+− | 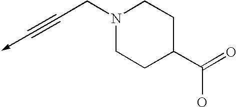 |
| 943 944 945 | rac+− | 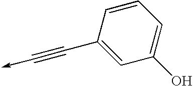 |
| 946 947 948 | rac+− | 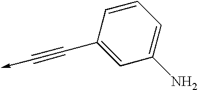 |
| 949 950 951 | rac+− | 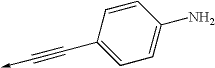 |
| 952 953 954 | rac+− | 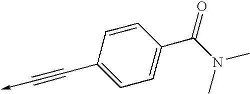 |
| 955 956 957 | rac+− | 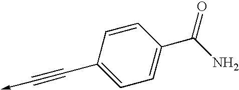 |

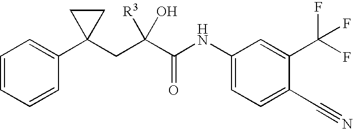
| | | |
|---|---|---|
| 958 959 960 | rac+− | 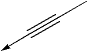 |
| 961 962 963 | rac+− | 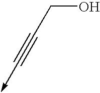 |
| 964 965 966 | rac+− | 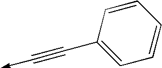 |
| 967 968 969 | rac+− | 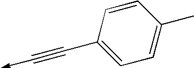 |
| 970 971 972 | rac+− | 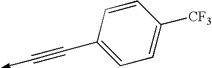 |
| 973 974 975 | rac+− | 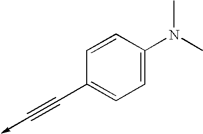 |
| 976 977 978 | rac+− | 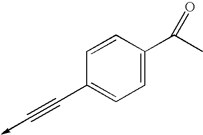 |
| 979 980 981 | rac+− | 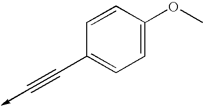 |
| 982 983 984 | rac+− | 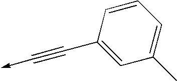 |
| 985 986 987 | rac+− | 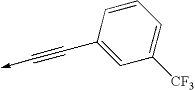 |
| 988 989 990 | rac+− | 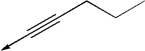 |
| 991 992 993 | rac+− | 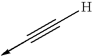 |
| 994 995 996 | rac+− | 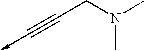 |
| 997 998 999 | rac+− | 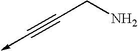 |
| 100010011002 | rac+− | 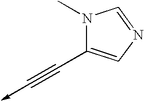 |
| 100310041005 | rac+− | 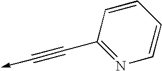 |
| 100610071008 | rac+− | 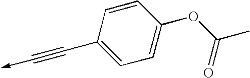 |
| 100910101011 | rac+− | 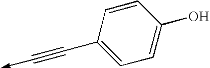 |
| 101210131014 | rac+− | 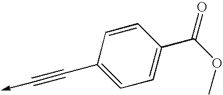 |
| 101510161017 | rac+− | 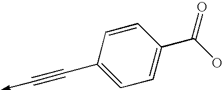 |
| 101810191020 | rac+− | 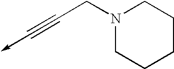 |
| 102110221023 | rac+− | 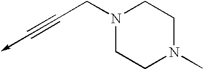 |
| 102410251026 | rac+− | 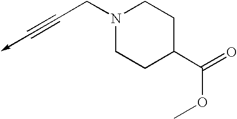 |
| 102710281029 | rac+− | 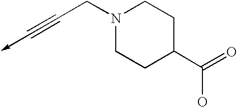 |
| 103010311032 | rac+− | 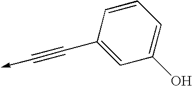 |
| 103310341035 | rac+− | 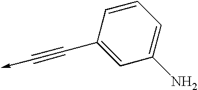 |
| 103610371038 | rac+− | 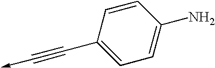 |
| 103910401041 | rac+− | 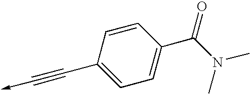 |
| 104210431044 | rac+− | 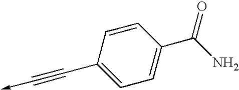 |

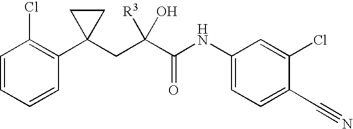
| | | |
|---|---|---|
| 104510461047 | rac+− | 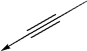 |
| 104810491050 | rac+− | 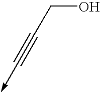 |
| 105110521053 | rac+− | 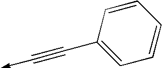 |
| 105410551056 | rac+− | 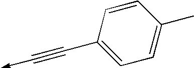 |
| 105710581059 | rac+− | 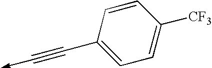 |
| 106010611062 | rac+− | 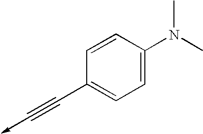 |
| 106310641065 | rac+− | 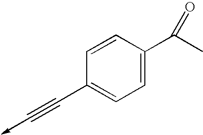 |
| 106610671068 | rac+− | 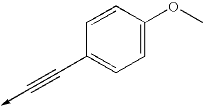 |
| 106910701071 | rac+− | 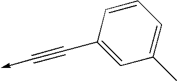 |
| 107210731074 | rac+− | 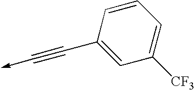 |
| 107510761077 | rac+− | 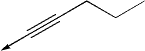 |
| 107810791080 | rac+− | 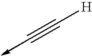 |
| 108110821083 | rac+− | 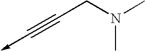 |
| 108410851086 | rac+− | 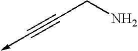 |
| 108710881089 | rac+− | 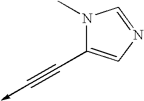 |
| 109010911092 | rac+− | 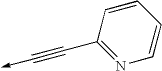 |
| 109310941095 | rac+− | 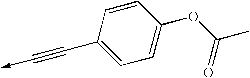 |
| 109610971098 | rac+− | 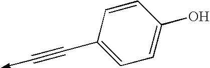 |
| 109911001101 | rac+− | 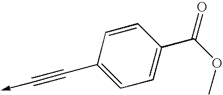 |
| 110211031104 | rac+− | 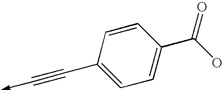 |
| 110511061107 | rac+− | 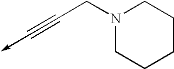 |
| 110811091110 | rac+− | 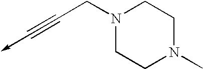 |
| 111111121113 | rac+− | 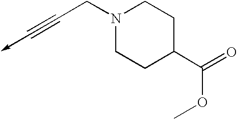 |
| 111411151116 | rac+− | 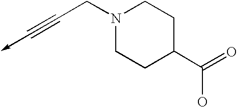 |
| 111711181119 | rac+− | 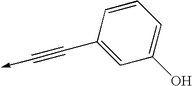 |
| 112011211122 | rac+− | 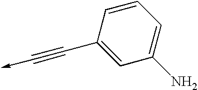 |
| 112311241125 | rac+− | 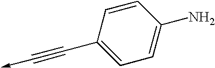 |
| 112611271128 | rac+− | 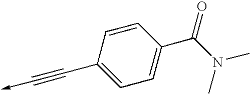 |
| 112911301131 | rac+− | 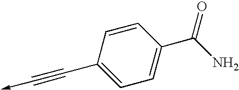 |

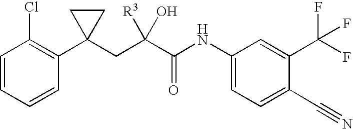
| | | |
|---|---|---|
| 113211331134 | rac+− | 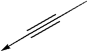 |
| 113511361137 | rac+− | 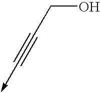 |
| 113811391140 | rac+− | 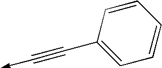 |
| 114111421143 | rac+− | 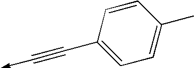 |
| 114411451146 | rac+− | 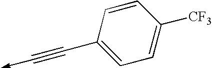 |
| 114711481149 | rac+− | 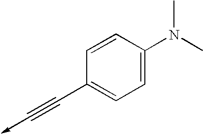 |
| 115011511152 | rac+− | 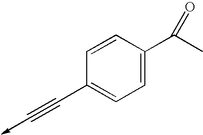 |
| 115311541155 | rac+− | 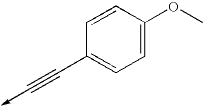 |
| 115611571158 | rac+− | 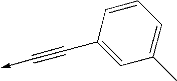 |
| 115911601161 | rac+− | 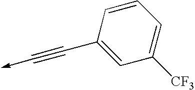 |
| 116211631164 | rac+− | 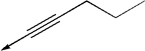 |
| 116511661167 | rac+− | 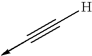 |
| 116811691170 | rac+− | 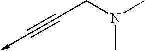 |
| 117111721173 | rac+− | 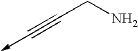 |
| 117411751176 | rac+− | 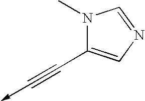 |
| 117711781179 | rac+− | 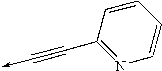 |
| 118011811182 | rac+− | 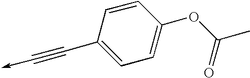 |
| 118311841185 | rac+− | 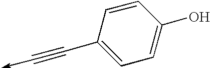 |
| 118611871188 | rac+− | 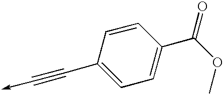 |
| 118911901191 | rac+− | 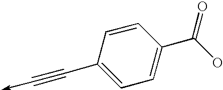 |
| 119211931194 | rac+− | 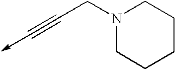 |
| 119511961197 | rac+− | 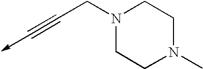 |
| 119811991200 | rac+− | 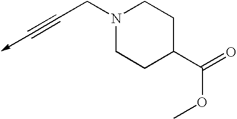 |
| 120112021203 | rac+− | 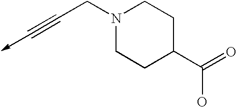 |
| 120412051206 | rac+− | 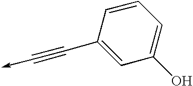 |
| 120712081209 | rac+− | 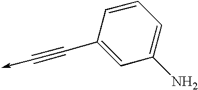 |
| 121012111212 | rac+− | 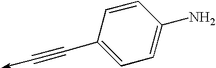 |
| 121312141215 | rac+− | 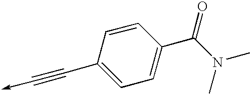 |
| 121612171218 | rac+− | 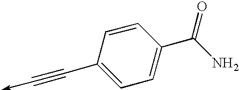 |

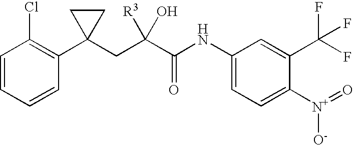
| | | |
|---|---|---|
| 121912201221 | rac+− | 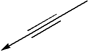 |
| 122212231224 | rac+− | 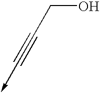 |
| 122512261227 | rac+− | 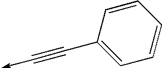 |
| 122812291230 | rac+− | 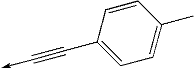 |
| 123112321233 | rac+− | 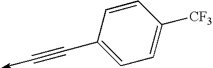 |
| 123412351236 | rac+− | 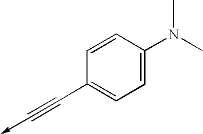 |
| 123712381239 | rac+− | 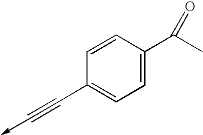 |
| 124012411242 | rac+− | 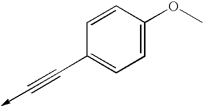 |
| 124312441245 | rac+− | 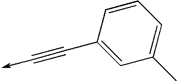 |
| 124612471248 | rac+− | 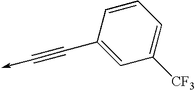 |
| 124912501251 | rac+− | 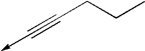 |
| 125212531254 | rac+− | 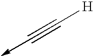 |
| 125512561257 | rac+− | 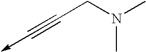 |
| 125812591260 | rac+− | 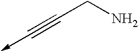 |
| 126112621263 | rac+− | 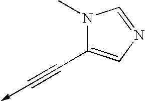 |
| 126412651266 | rac+− | 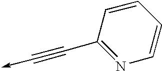 |
| 126712681269 | rac+− | 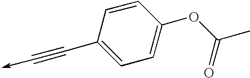 |
| 127012711272 | rac+− | 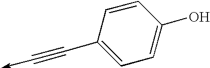 |
| 127312741275 | rac+− | 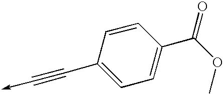 |
| 127612771278 | rac+− | 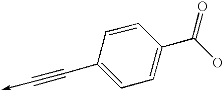 |
| 127912801281 | rac+− | 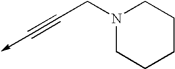 |
| 128212831284 | rac+− | 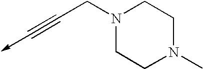 |
| 128512861287 | rac+− | 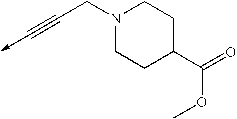 |
| 128812891290 | rac+− | 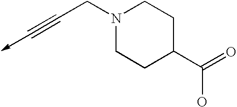 |
| 129112921293 | rac+− | 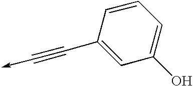 |
| 129412951296 | rac+− | 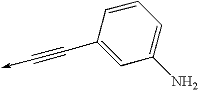 |
| 129712981299 | rac+− | 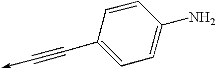 |
| 130013011302 | rac+− | 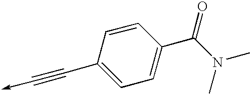 |
| 130313041305 | rac+− | 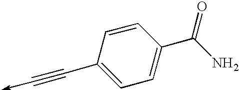 |

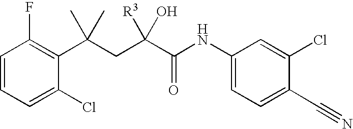
| | | |
|---|---|---|
| 130613071308 | rac+− | 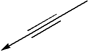 |
| 130913101311 | rac+− | 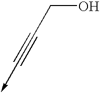 |
| 131213131314 | rac+− | 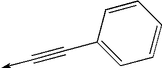 |
| 131513161317 | rac+− | 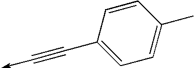 |
| 131813191320 | rac+− | 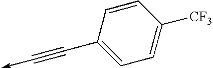 |
| 132113221323 | rac+− | 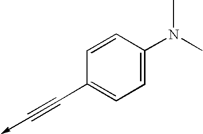 |
| 132413251326 | rac+− | 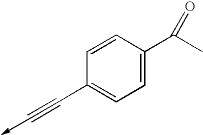 |
| 132713281329 | rac+− | 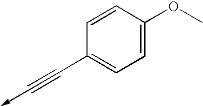 |
| 133013311332 | rac+− | 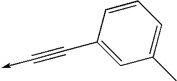 |
| 133313341335 | rac+− | 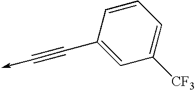 |
| 133613371338 | rac+− | 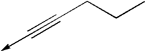 |
| 133913401341 | rac+− | 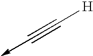 |
| 134213431344 | rac+− | 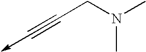 |
| 134513461347 | rac+− | 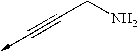 |
| 134813491350 | rac+− | 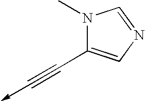 |
| 135113521353 | rac+− | 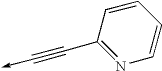 |
| 135413551356 | rac+− | 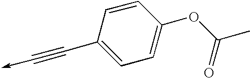 |
| 135713581359 | rac+− | 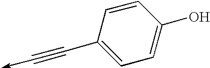 |
| 136013611362 | rac+− | 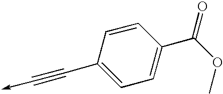 |
| 136313641365 | rac+− | 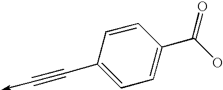 |
| 136613671368 | rac+− | 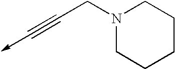 |
| 136913701371 | rac+− | 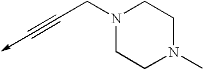 |
| 137213731374 | rac+− | 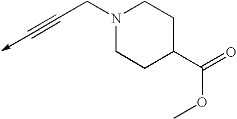 |
| 137513761377 | rac+− | 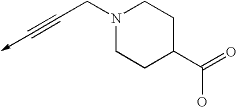 |
| 137813791380 | rac+− | 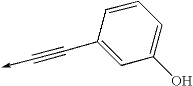 |
| 138113821383 | rac+− | 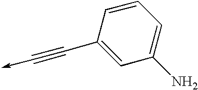 |
| 138413851386 | rac+− | 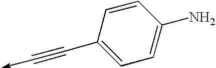 |
| 138713881389 | rac+− | 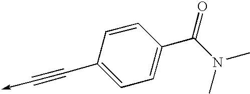 |
| 139013911392 | rac+− | 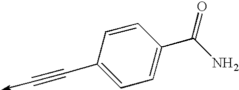 |

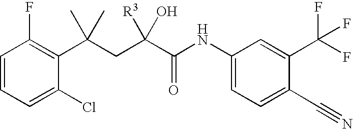
| | | |
|---|---|---|
| 139313941395 | rac+− | 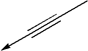 |
| 139613971398 | rac+− | 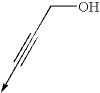 |
| 139914001401 | rac+− | 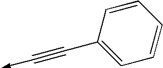 |
| 140214031404 | rac+− | 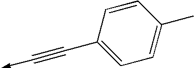 |
| 140514061407 | rac+− | 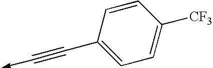 |
| 140814091410 | rac+− | 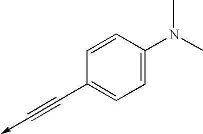 |
| 141114121413 | rac+− | 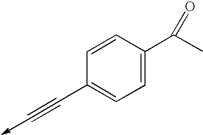 |
| 141414151416 | rac+− | 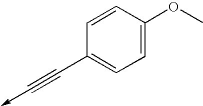 |
| 141714181419 | rac+− | 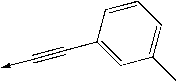 |
| 142014211422 | rac+− | 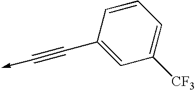 |
| 142314241425 | rac+− | 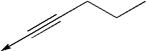 |
| 142614271428 | rac+− | 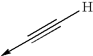 |
| 142914301431 | rac+− | 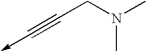 |
| 143214331434 | rac+− | 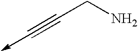 |
| 143514361437 | rac+− | 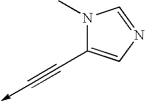 |
| 143814391440 | rac+− | 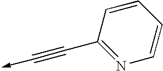 |
| 144114421443 | rac+− | 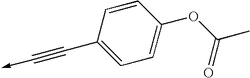 |
| 144414451446 | rac+− | 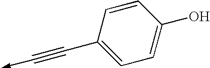 |
| 144714481449 | rac+− | 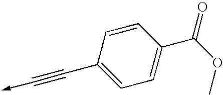 |
| 145014511452 | rac+− | 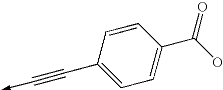 |
| 145314541455 | rac+− | 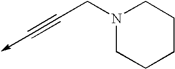 |
| 145614571458 | rac+− | 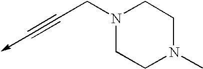 |
| 145914601461 | rac+− | 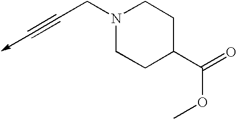 |
| 146214631464 | rac+− | 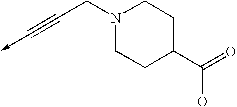 |
| 146514661467 | rac+− | 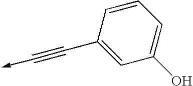 |
| 146814691470 | rac+− | 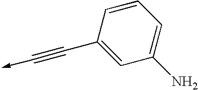 |
| 147114721473 | rac+− | 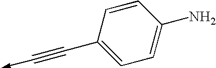 |
| 147414751476 | rac+− | 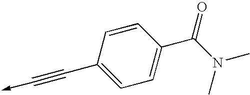 |
| 147714781479 | rac+− | 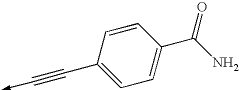 |

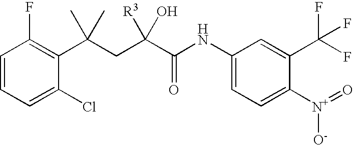
| | | |
|---|---|---|
| 148014811482 | rac+− | 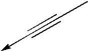 |
| 148314841485 | rac+− | 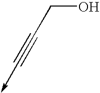 |
| 148614871488 | rac+− | 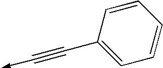 |
| 148914901491 | rac+− | 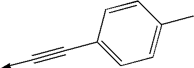 |
| 149214931494 | rac+− | 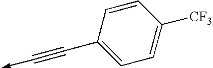 |
| 149514961497 | rac+− | 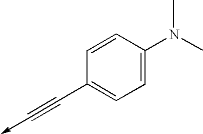 |
| 149814991500 | rac+− | 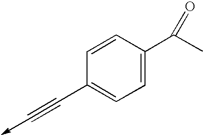 |
| 150115021503 | rac+− | 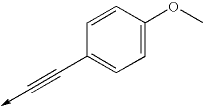 |
| 150415051506 | rac+− | 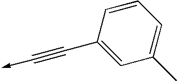 |
| 150715081509 | rac+− | 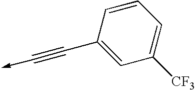 |
| 151015111512 | rac+− | 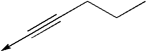 |
| 151315141515 | rac+− | 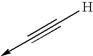 |
| 151615171518 | rac+− | 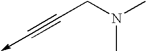 |
| 151915201521 | rac+− | 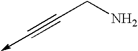 |
| 152215231524 | rac+− | 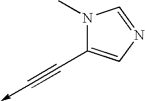 |
| 152515261527 | rac+− | 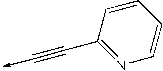 |
| 152815291530 | rac+− | 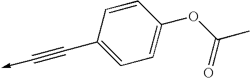 |
| 153115321533 | rac+− | 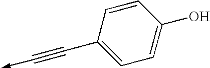 |
| 153415351536 | rac+− | 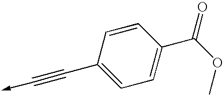 |
| 153715381539 | rac+− | 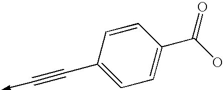 |
| 154015411542 | rac+− | 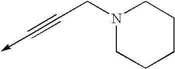 |
| 154315441545 | rac+− | 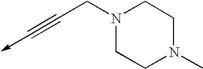 |
| 154615471548 | rac+− | 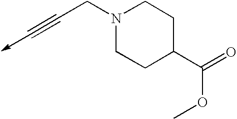 |
| 154915501551 | rac+− | 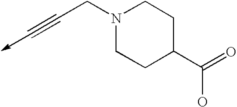 |
| 155215531554 | rac+− | 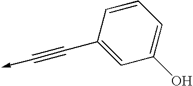 |
| 155515561557 | rac+− | 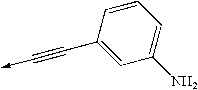 |
| 155815591560 | rac+− | 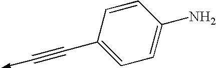 |
| 156115621563 | rac+− | 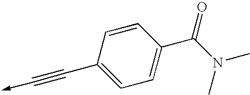 |
| 156415651566 | rac+− | 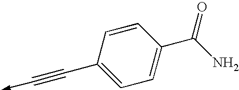 |

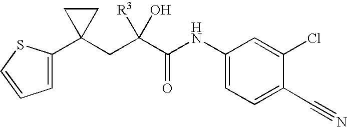
| | | |
|---|---|---|
| 156715681569 | rac+− | 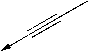 |
| 157015711572 | rac+− | 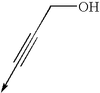 |
| 157315741575 | rac+− | 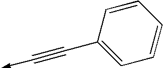 |
| 157615771578 | rac+− | 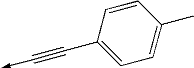 |
| 157915801581 | rac+− | 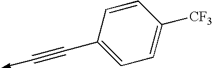 |
| 158215831584 | rac+− | 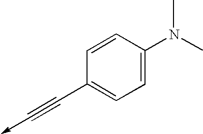 |
| 158515861587 | rac+− | 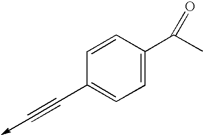 |
| 158815891590 | rac+− | 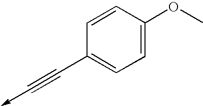 |
| 159115921593 | rac+− | 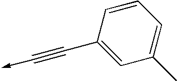 |
| 159415951596 | rac+− | 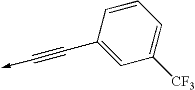 |
| 159715981599 | rac+− | 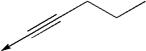 |
| 160016011602 | rac+− | 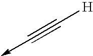 |
| 160316041605 | rac+− | 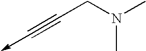 |
| 160616071608 | rac+− | 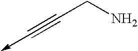 |
| 160916101611 | rac+− | 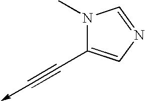 |
| 161216131614 | rac+− | 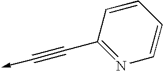 |
| 161516161617 | rac+− | 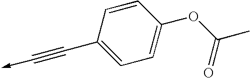 |
| 161816191620 | rac+− | 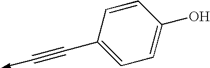 |
| 162116221623 | rac+− | 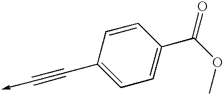 |
| 162416251626 | rac+− | 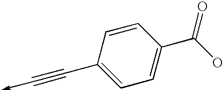 |
| 162716281629 | rac+− | 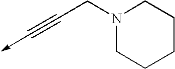 |
| 163016311632 | rac+− | 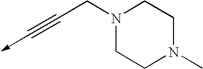 |
| 163316341635 | rac+− | 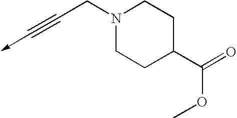 |
| 163616371638 | rac+− | 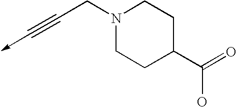 |
| 163916401641 | rac+− | 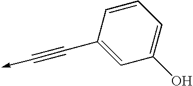 |
| 164216431644 | rac+− | 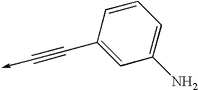 |
| 164516461647 | rac+− | 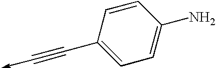 |
| 164816491650 | rac+− | 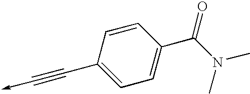 |
| 165116521653 | rac+− | 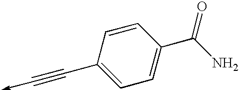 |

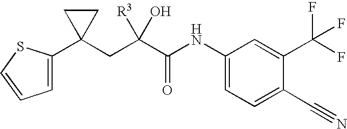
| | | |
|---|---|---|
| 165416551656 | rac+− | 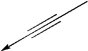 |
| 165716581659 | rac+− | 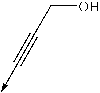 |
| 166016611662 | rac+− | 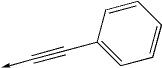 |
| 166316641665 | rac+− | 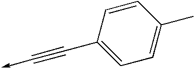 |
| 166616671668 | rac+− | 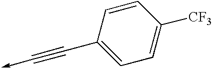 |
| 166916701671 | rac+− | 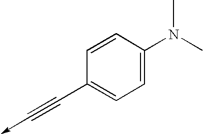 |
| 167216731674 | rac+− | 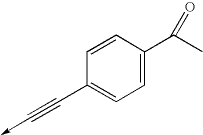 |
| 167516761677 | rac+− | 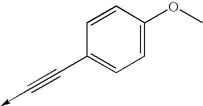 |
| 167816791680 | rac+− | 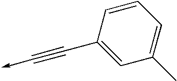 |
| 168116821683 | rac+− | 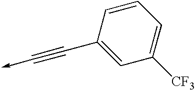 |
| 168416851686 | rac+− | 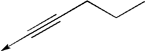 |
| 168716881689 | rac+− | 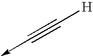 |
| 169016911692 | rac+− | 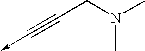 |
| 169316941695 | rac+− | 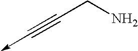 |
| 169616971698 | rac+− | 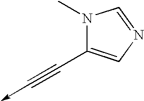 |
| 169917001701 | rac+− | 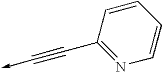 |
| 170217031704 | rac+− | 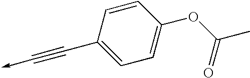 |
| 170517061707 | rac+− | 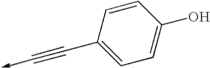 |
| 170817091710 | rac+− | 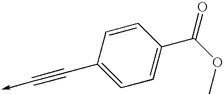 |
| 171117121713 | rac+− | 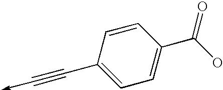 |
| 171417151716 | rac+− | 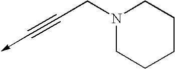 |
| 171717181719 | rac+− | 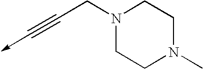 |
| 172017211722 | rac+− | 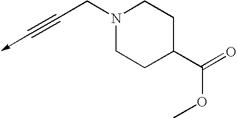 |
| 172317241725 | rac+− | 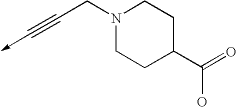 |
| 172617271728 | rac+− | 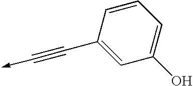 |
| 172917301731 | rac+− | 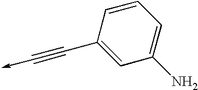 |
| 173217331734 | rac+− | 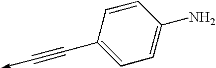 |
| 173517361737 | rac+− | 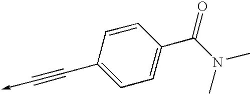 |
| 173817391740 | rac+− | 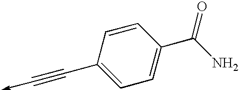 |

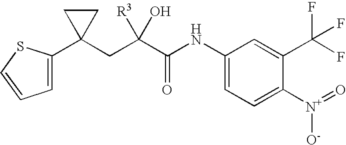
| | | |
|---|---|---|
| 174117421743 | rac+− | 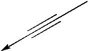 |
| 174417451746 | rac+− | 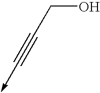 |
| 174717481749 | rac+− | 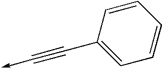 |
| 175017511752 | rac+− | 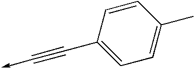 |
| 175317541755 | rac+− | 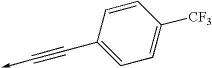 |
| 175617571758 | rac+− | 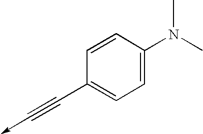 |
| 175917601761 | rac+− | 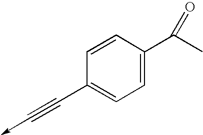 |
| 176217631764 | rac+− | 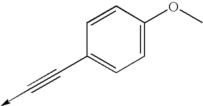 |
| 176517661767 | rac+− | 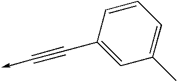 |
| 176817691770 | rac+− | 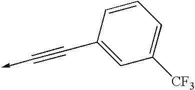 |
| 177117721773 | rac+− | 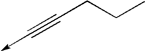 |
| 177417751776 | rac+− | 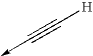 |
| 177717781779 | rac+− | 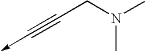 |
| 178017811782 | rac+− | 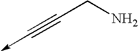 |
| 178317841785 | rac+− | 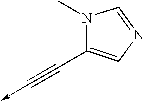 |
| 178617871788 | rac+− | 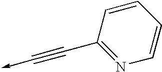 |
| 178917901791 | rac+− | 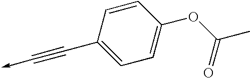 |
| 179217931794 | rac+− | 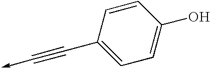 |
| 179517961797 | rac+− | 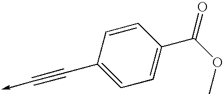 |
| 179817991800 | rac+− | 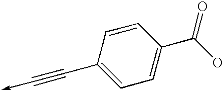 |
| 180118021803 | rac+− | 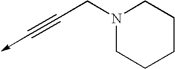 |
| 180418051806 | rac+− | 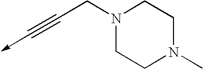 |
| 180718081809 | rac+− | 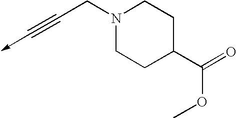 |
| 181018111812 | rac+− | 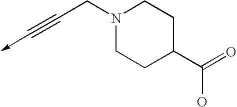 |
| 181318141815 | rac+− | 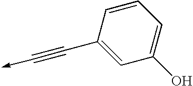 |
| 181618171818 | rac+− | 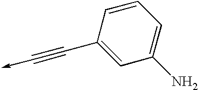 |
| 181918201821 | rac+− | 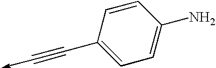 |
| 182218231824 | rac+− | 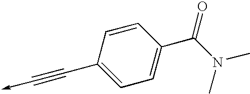 |
| 182518261827 | rac+− | 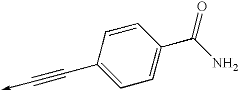 |

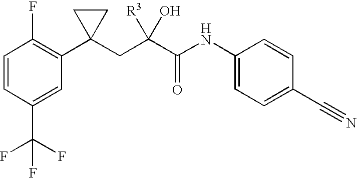
| | | |
|---|---|---|
| 182818291830 | rac+− | 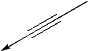 |
| 183118321833 | rac+− | 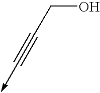 |
| 183418351836 | rac+− | 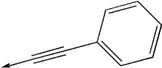 |
| 183718381839 | rac+− | 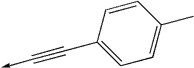 |
| 184018411842 | rac+− | 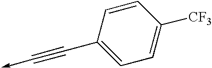 |
| 184318441845 | rac+− | 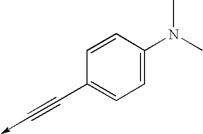 |
| 184618471848 | rac+− | 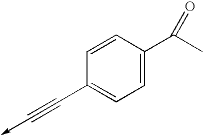 |
| 184918501851 | rac+− | 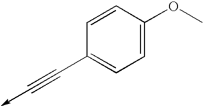 |
| 185218531854 | rac+− | 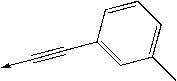 |
| 185518561857 | rac+− | 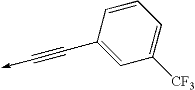 |
| 185818591860 | rac+− | 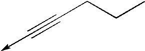 |
| 186118621863 | rac+− | 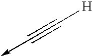 |
| 186418651866 | rac+− | 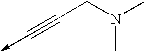 |
| 186718681869 | rac+− | 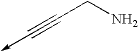 |
| 187018711872 | rac+− | 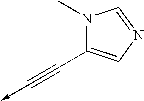 |
| 187318741875 | rac+− | 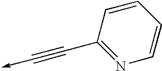 |
| 187618771878 | rac+− | 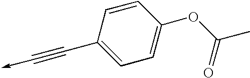 |
| 187918801881 | rac+− | 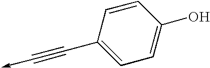 |
| 188218831884 | rac+− | 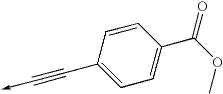 |
| 188518861887 | rac+− | 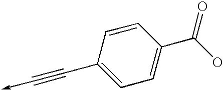 |
| 188818891890 | rac+− | 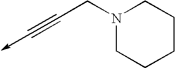 |
| 189118921893 | rac+− | 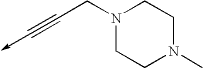 |
| 189418951896 | rac+− | 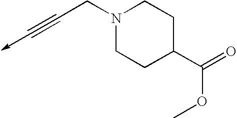 |
| 189718981899 | rac+− | 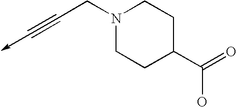 |
| 190019011902 | rac+− | 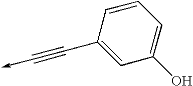 |
| 190319041905 | rac+− | 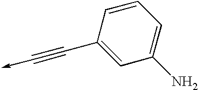 |
| 190619071908 | rac+− | 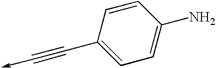 |
| 190919101911 | rac+− | 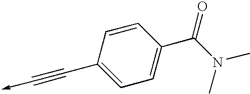 |
| 191219131914 | rac+− | 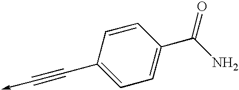 |

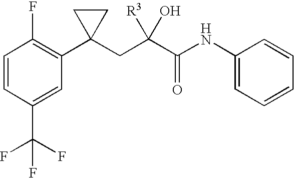
| | | |
|---|---|---|
| 191519161917 | rac+− | 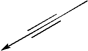 |
| 191819191920 | rac+− | 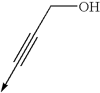 |
| 192119221923 | rac+− | 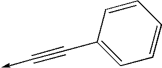 |
| 192419251926 | rac+− | 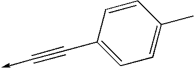 |
| 192719281929 | rac+− | 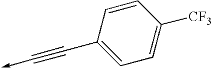 |
| 193019311932 | rac+− | 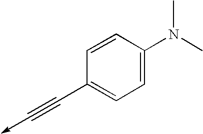 |
| 193319341935 | rac+− | 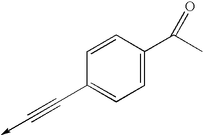 |
| 193619371938 | rac+− | 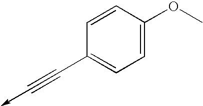 |
| 193919401941 | rac+− | 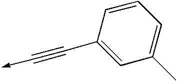 |
| 194219431944 | rac+− | 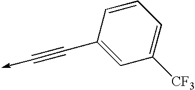 |
| 194519461947 | rac+− | 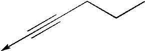 |
| 194819491950 | rac+− | 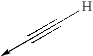 |
| 195119521953 | rac+− | 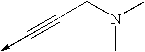 |
| 195419551956 | rac+− | 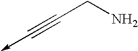 |
| 195719581959 | rac+− | 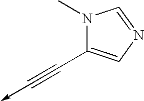 |
| 196019611962 | rac+− | 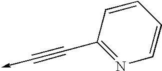 |
| 196319641965 | rac+− | 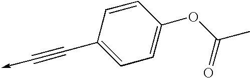 |
| 196619671968 | rac+− | 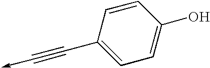 |
| 196919701971 | rac+− | 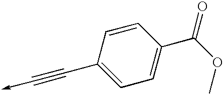 |
| 197219731974 | rac+− | 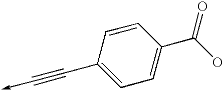 |
| 197519761977 | rac+− | 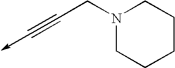 |
| 197819791980 | rac+− | 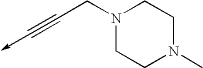 |
| 198119821983 | rac+− | 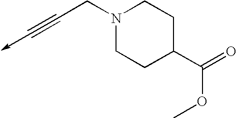 |
| 198419851986 | rac+− | 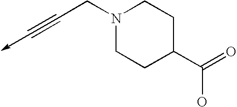 |
| 198719881989 | rac+− | 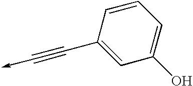 |
| 199019911992 | rac+− | 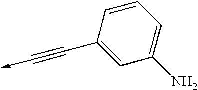 |
| 199319941995 | rac+− | 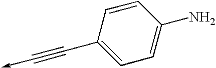 |
| 199619971998 | rac+− | 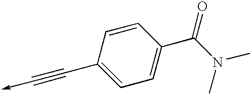 |
| 199920002001 | rac+− | 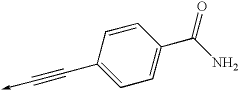 |

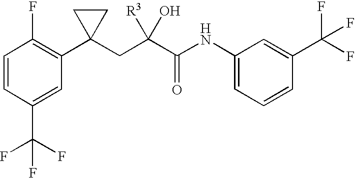
| | | |
|---|---|---|
| 200220032004 | rac+− | 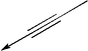 |
| 200520062007 | rac+− | 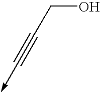 |
| 200820092010 | rac+− | 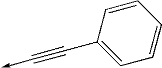 |
| 201120122013 | rac+− | 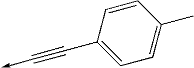 |
| 201420152016 | rac+− | 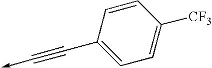 |
| 201720182019 | rac+− | 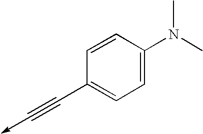 |
| 202020212022 | rac+− | 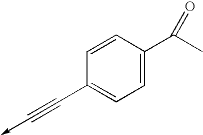 |
| 202320242025 | rac+− | 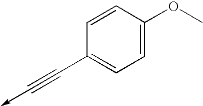 |
| 202620272028 | rac+− | 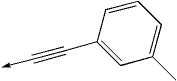 |
| 202920302031 | rac+− | 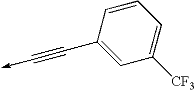 |
| 203220332034 | rac+− | 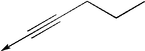 |
| 203520362037 | rac+− | 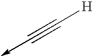 |
| 203820392040 | rac+− | 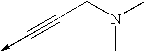 |
| 204120422043 | rac+− | 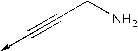 |
| 204420452046 | rac+− | 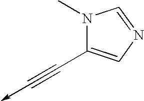 |
| 204720482049 | rac+− | 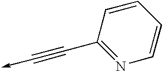 |
| 205020512052 | rac+− | 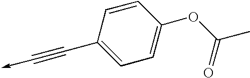 |
| 205320542055 | rac+− | 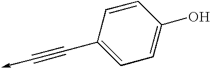 |
| 205620572058 | rac+− | 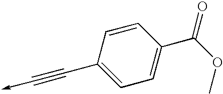 |
| 205920602061 | rac+− | 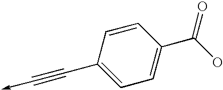 |
| 206220632064 | rac+− | 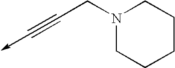 |
| 206520662067 | rac+− | 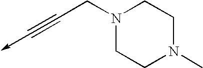 |
| 206820692070 | rac+− | 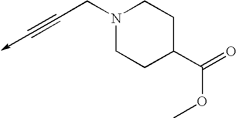 |
| 207120722073 | rac+− | 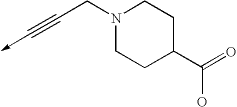 |
| 207420752076 | rac+− | 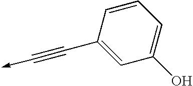 |
| 207720782079 | rac+− | 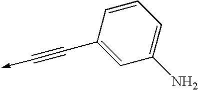 |
| 208020812082 | rac+− | 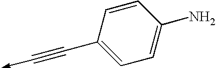 |
| 208320842085 | rac+− | 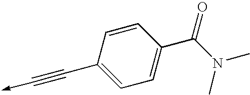 |
| 208620872088 | rac+− | 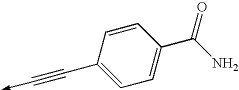 |

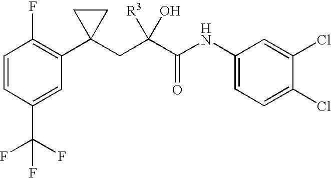
| | | |
|---|---|---|
| 208920902091 | rac+− | 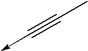 |
| 209220932094 | rac+− | 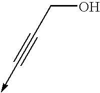 |
| 209520962097 | rac+− | 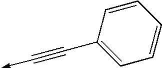 |
| 209820992100 | rac+− | 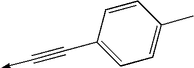 |
| 210121022103 | rac+− | 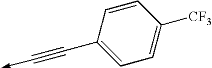 |
| 210421052106 | rac+− | 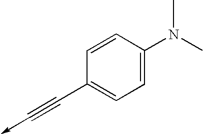 |
| 210721082109 | rac+− | 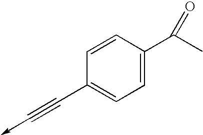 |
| 211021112112 | rac+− | 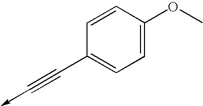 |
| 211321142115 | rac+− | 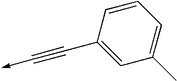 |
| 211621172118 | rac+− | 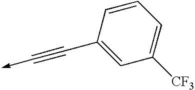 |
| 211921202121 | rac+− | 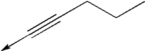 |
| 212221232124 | rac+− | 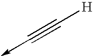 |
| 212521262127 | rac+− | 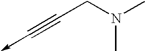 |
| 212821292130 | rac+− | 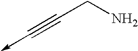 |
| 213121322133 | rac+− | 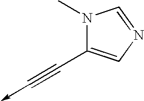 |
| 213421352136 | rac+− | 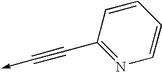 |
| 213721382139 | rac+− | 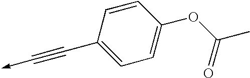 |
| 214021412142 | rac+− | 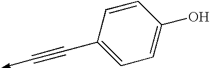 |
| 214321442145 | rac+− | 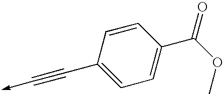 |
| 214621472148 | rac+− | 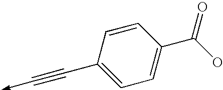 |
| 214921502151 | rac+− | 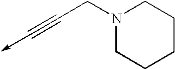 |
| 215221532154 | rac+− | 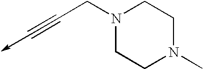 |
| 215521562157 | rac+− | 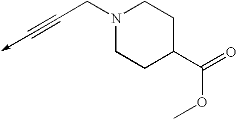 |
| 215821592160 | rac+− | 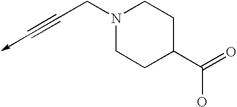 |
| 216121622163 | rac+− | 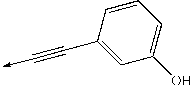 |
| 216421652166 | rac+− | 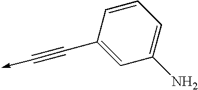 |
| 216721682169 | rac+− | 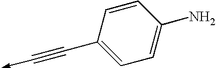 |
| 217021712172 | rac+− | 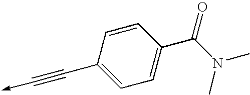 |
| 217321742175 | rac+− | 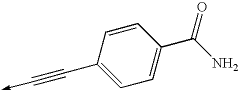 |

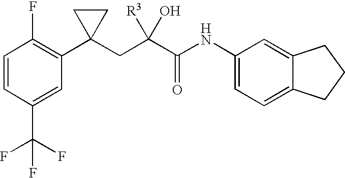
| | | |
|---|---|---|
| 217621772178 | rac+− | 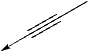 |
| 217921802181 | rac+− | 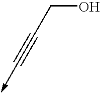 |
| 218221832184 | rac+− | 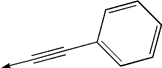 |
| 218521862187 | rac+− | 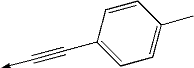 |
| 218821892190 | rac+− | 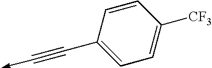 |
| 219121922193 | rac+− | 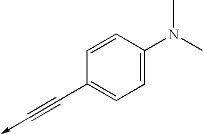 |
| 219421952196 | rac+− | 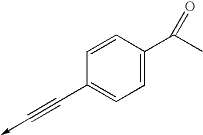 |
| 219721982199 | rac+− | 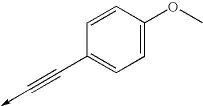 |
| 220022012202 | rac+− | 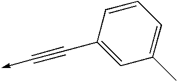 |
| 220322042205 | rac+− | 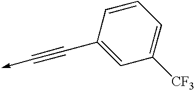 |
| 220622072208 | rac+− | 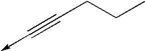 |
| 220922102211 | rac+− | 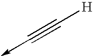 |
| 221222132214 | rac+− | 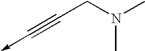 |
| 221522162217 | rac+− | 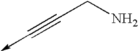 |
| 221822192220 | rac+− | 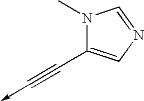 |
| 222122222223 | rac+− | 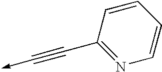 |
| 222422252226 | rac+− | 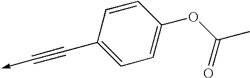 |
| 222722282229 | rac+− | 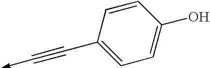 |
| 223022312232 | rac+− | 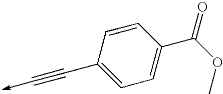 |
| 223322342235 | rac+− | 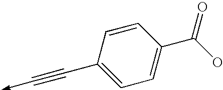 |
| 223622372238 | rac+− | 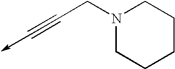 |
| 223922402241 | rac+− | 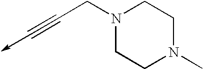 |
| 224222432244 | rac+− | 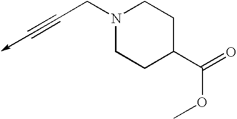 |
| 224522462247 | rac+− | 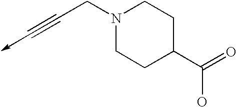 |
| 224822492250 | rac+− | 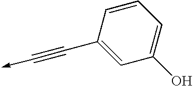 |
| 225122522253 | rac+− | 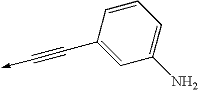 |
| 225422552256 | rac+− | 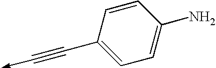 |
| 225722582259 | rac+− | 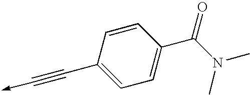 |
| 226022612262 | rac+− | 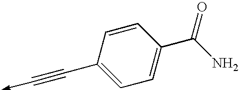 |

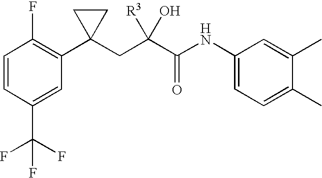
| | | |
|---|---|---|
| 226322642265 | rac+− | 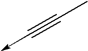 |
| 226622672268 | rac+− | 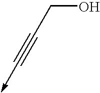 |
| 226922702271 | rac+− | 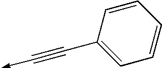 |
| 227222732274 | rac+− | 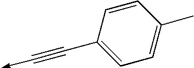 |
| 227522762277 | rac+− | 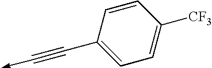 |
| 227822792280 | rac+− | 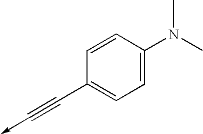 |
| 228122822283 | rac+− | 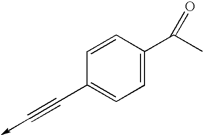 |
| 228422852286 | rac+− | 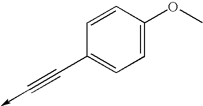 |
| 228722882289 | rac+− | 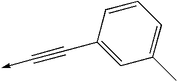 |
| 229022912292 | rac+− | 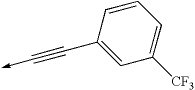 |
| 229322942295 | rac+− | 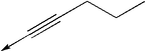 |
| 229622972298 | rac+− | 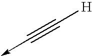 |
| 229923002301 | rac+− | 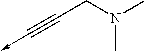 |
| 230223032304 | rac+− | 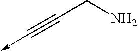 |
| 230523062307 | rac+− | 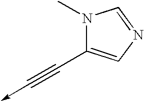 |
| 230823092310 | rac+− | 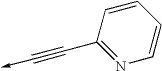 |
| 231123122313 | rac+− | 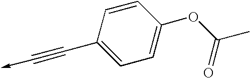 |
| 231423152316 | rac+− | 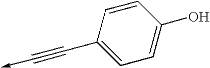 |
| 231723182319 | rac+− | 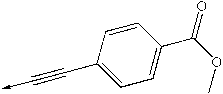 |
| 232023212322 | rac+− | 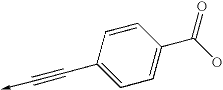 |
| 232323242325 | rac+− | 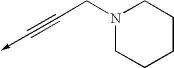 |
| 232623272328 | rac+− | 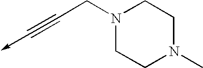 |
| 232923302331 | rac+− | 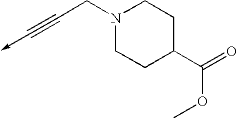 |
| 233223332334 | rac+− | 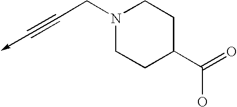 |
| 233523362337 | rac+− | 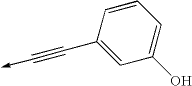 |
| 233823392340 | rac+− | 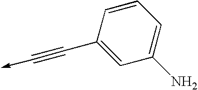 |
| 234123422343 | rac+− | 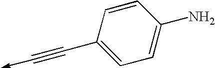 |
| 234423452346 | rac+− | 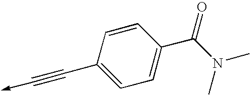 |
| 234723482349 | rac+− | 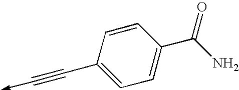 |

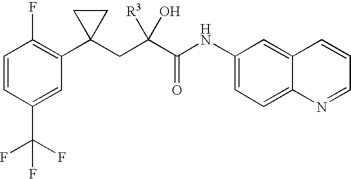
| | | |
|---|---|---|
| 235023512352 | rac+− | 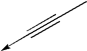 |
| 235323542355 | rac+− | 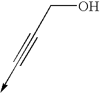 |
| 235623572358 | rac+− | 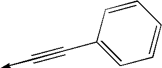 |
| 235923602361 | rac+− | 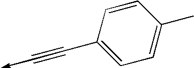 |
| 236223632364 | rac+− | 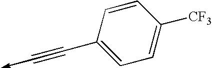 |
| 236523662367 | rac+− | 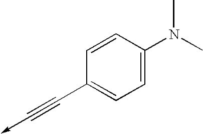 |
| 236823692370 | rac+− | 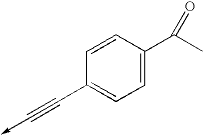 |
| 237123722373 | rac+− | 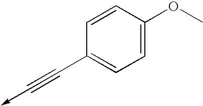 |
| 237423752376 | rac+− | 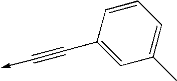 |
| 237723782379 | rac+− | 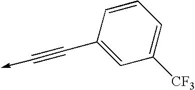 |
| 238023812382 | rac+− | 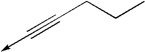 |
| 238323842385 | rac+− | 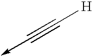 |
| 238623872388 | rac+− | 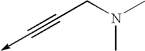 |
| 238923902391 | rac+− | 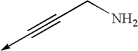 |
| 239223932394 | rac+− | 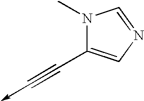 |
| 239523962397 | rac+− | 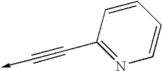 |
| 239823992400 | rac+− | 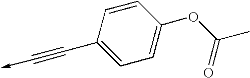 |
| 240124022403 | rac+− | 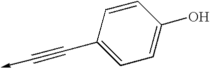 |
| 240424052406 | rac+− | 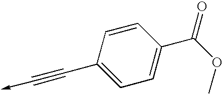 |
| 240724082409 | rac+− | 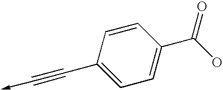 |
| 241024112412 | rac+− | 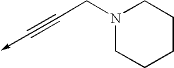 |
| 241324142415 | rac+− | 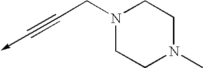 |
| 241624172418 | rac+− | 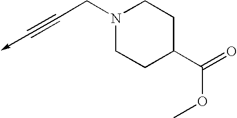 |
| 241924202421 | Rac+− | 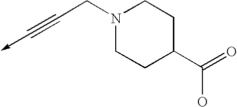 |
| 242224232424 | rac+− | 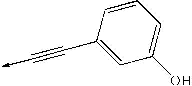 |
| 242524262427 | rac+− | 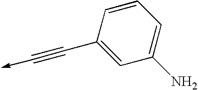 |
| 242824292430 | rac+− | 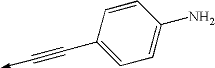 |
| 243124322433 | rac+− | 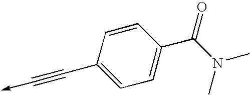 |
| 243424352436 | rac+− | 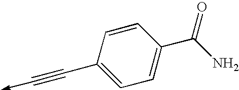 |

18. Pharmaceutical composition comprising at least one compound of the general formula I according to claim 1 and, where appropriate, at least one further active ingredient together with pharmaceutically suitable excipients and/or carriers.

19. Pharmaceutical composition according to claim 18, where the further active ingredient is a SERM (selective estrogen receptor modulator), an estrogen, estrogen derivative or a substance having estrogenic activity, an aromatase inhibitor, antiestrogen or a prostaglandin.

20. Pharmaceutical composition according to claim 19, where the following estrogen derivatives are suitable: 17□-estradiol 3-alkylsulphonates, 17□-ethinylestradiol 3-alkylsulphonates, 17□-estradiol 3- or 17-esters, 17□-ethinylestradiol 3-ethers.

21. Pharmaceutical composition according to claim 19, where the further active ingredients may be tamoxifen, 5-(4-{5-[(RS)-(4,4,5,5,5-pentafluoropentyl)sulphinyl]pentyloxy}phenyl)-6-phenyl-8,9-dihydro-7H-benzocyclohepten-2-ol, ICI 182 780 (7alpha-[9-(4,4,5,5-pentafluoropentylsulphinyl)nonyl]estra-1,3,5(10)-triene-3,17-beta-diol), 11beta-fluoro-7alpha-[5-(methyl{3-[(4,4,5,5,5-pentafluoropentyl)sulphanyl]-propyl}amino)pentyl]estra-1,3,5(10)-triene-3,17beta-diol, 11beta-fluoro-7alpha-{5-[methyl(7,7,8,8,9,9,10,10,10-nonafluorodecyl)amino]pentyl}estra-1,3,5(10)-triene-3,17beta-diol, 11beta-fluoro-17alpha-methyl-7alpha-{5-[methyl(8,8,9,9,9-pentafluorononyl)amino]pentyl}estra-1,3,5(10)-triene-3,17beta-diol, clomifene, raloxifene, fadrozole, formestane, letrozole, anastrozole, atamestane, 17□-estradiol, 17□-ethinylestradiol, estriol, 17□-estradiol 3-isopropylsulphonate, 17□-ethinylestradiol-propylsulphonate (turisterone), estradiol 3-benzoate, estradiol 17-valerate, 17□-ethinylestradiol 3-methyl ether (mestranol) or conjugated equine estrogens (CEE).

22. Compounds according to claim 1 for producing a medicament.

23. A method for the therapy and/or prophylaxis of gynaecological disorders such as endometriosis, leiomyomas of the uterus, dysfunctional bleeding and dysmenorrhoea, comprising administering to a host in need thereof a compound of claim 1.

24. Use of compounds according to claim 1 for producing a medicament for the therapy and/or prophylaxis of hormone-dependent tumours.

25. Use of compounds according to claim 1 for producing a medicament for the therapy and/or prophylaxis of breast carcinomas.

26. Use of compounds according to claim 1 for producing a medicament for the therapy and/or prophylaxis of endometrial carcinoma.

27. Use of compounds according to claim 1 for producing a medicament for the therapy and/or prophylaxis of ovarian carcinomas.

28. Use of compounds according to claim 1 for producing a medicament for the therapy and/or prophylaxis of prostate carcinomas.

29. Use of compounds according to claim 1 for producing a medicament for female hormone replacement therapy.

30. Use of compounds according to claim 1 for female fertility control.
